SEQUENCE LISTING (20 sequences)

SEQ ID NO: 1 - DNA, 21 bp, Mus musculus
tctcaagactgttggttgctg

SEQ ID NO: 2 - DNA, 22 bp, Mus musculus
tgaacaaccaatgatagcacag

SEQ ID NO: 3 - DNA, 20 bp, Mus musculus
ctcactgtgctggcctatga

SEQ ID NO: 4 - DNA, 21 bp, Mus musculus
gggttgtacacagtgctcgat

SEQ ID NO: 5 - DNA, 20 bp, Mus musculus
actggcgagaactggatgac

SEQ ID NO: 6 - DNA, 20 bp, Mus musculus
gccatcaccccttaacagaa

SEQ ID NO: 7 - DNA, 20 bp, Mus musculus
tggccgtttcttaagacgaa

SEQ ID NO: 8 - DNA, 20 bp, Mus musculus
ctcaatttccctgctgctgt

SEQ ID NO: 9 - DNA, 20 bp, Mus musculus
ccctagttgggactgtgacc

SEQ ID NO: 10 - DNA, 20 bp, Mus musculus
agctctgttcaccccacaag

SEQ ID NO: 11 - DNA, 20 bp, Mus musculus
ggagtcaggccaacatgagt

SEQ ID NO: 12 - DNA, 22 bp, Mus musculus
tgcagaggaattgtaatggaga

SEQ ID NO: 13 - DNA, 20 bp, Mus musculus
cttctagtcgggcaagatgc

SEQ ID NO: 14 - DNA, 20 bp, Mus musculus
gggtctgttgtcgttctggt

SEQ ID NO: 15 - DNA, 19 bp, Mus musculus
gcccaggtttcgacagact

SEQ ID NO: 16 - DNA, 24 bp, Mus musculus
ccactagtgatagaaagctctgga

SEQ ID NO: 17 - DNA, 20 bp, Mus musculus
caagccctattcaggagcag

SEQ ID NO: 18 - DNA, 20 bp, Mus musculus
cttggggtccactgtgaagt

SEQ ID NO: 19 - DNA, 20 bp, Mus musculus
agcagttcgctgttgtgatg

SEQ ID NO: 20 - DNA, 20 bp, Mus musculus
gcacgtttctccttttctgc

(19) United States
(12) Patent Application Publication
(10) Pub. No.: US 2005/0266563 A1
(43) Pub. Date: Dec. 1, 2005

(54) Methods and compositions relating to neuronal cell and tissue differentiation

(75) Inventors: Jeffrey D. Macklis, Brookline, MA (US); Paola Arlotta, Somerville, MA (US); Bradley J. Molyneaux, Cambridge, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: The General Hospital Corporation, Boston, MA (US)

(21) Appl. No.: 11/085,839

(22) Filed: Mar. 21, 2005

Related U.S. Application Data
(60) Provisional application No. 60/554,598, filed on Mar. 19, 2004.

(51) Int. Cl. C12N 5/08; C12N 15/85

(52) U.S. Cl. 435/455; 435/368

(57) ABSTRACT

The invention relates to methods for isolating and purifying specific types of neurons, such as cortical or other projection neurons including corticospinal motor neurons, subcerebral projection neurons, and callosal projection neurons. The invention also relates to genes that are specific for particular neuronal subtypes, and the use of such genes in genetic/molecular control of cell development. The isolated cells and subtype-specific genes also have uses in diagnostics, therapeutics, and screening assays for pharmaceutical molecules.

### RELATED APPLICATIONS

This application claims the benefit under 35 U.S.C. § 1 1 9(e) of U.S. provisional application 60/554,598, filed Mar. 19, 2004, which is incorporated by reference herein.

### GOVERNMENT SUPPORT

This work was partially supported by the National Institutes of Health under grant numbers NS41590, NS42190 and MRRC HD18655 and training grant number 5T32 AG00222-11. The government may have certain rights in this invention.

### FIELD OF THE INVENTION

The invention relates to methods for isolating and purifying specific types of neurons, such as cortical or other projection neurons including corticospinal motor neurons, subcerebral projection neurons, and callosal projection neurons. The invention also relates to genes that are specific for particular neuronal subtypes, and the use of such genes in genetic/molecular control of cell development. The isolated cells and subtype-specific genes also have uses in diagnostics, therapeutics, and screening assays for pharmaceutical molecules.

### BACKGROUND OF THE INVENTION

During the development of the central nervous system, neuronal progenitors undergo precise step-wise differentiation to ultimately produce the complex variety of neuronal subtypes that populate the mature brain. Extensive work has progressively unraveled the molecular mechanisms controlling processes of early neuronal specification, and has identified pro-neuronal transcription factors and fate determination genes that mediate early aspects of neurogenesis and neuronal differentiation in several regions of the CNS (Edlund and Jessell, 1999; Bertrand et al., 2002). In contrast, much less is known about the genetic programs controlling the later specification and differentiation of distinct neuronal subtypes, and how these molecular events relate to the general programs of neurogenesis in the CNS.

Recent success in elucidating the genetic determinants of neuronal subtype specification has been limited to distinct regions of the mammalian CNS, principally the spinal cord (Jessell, 2000) and retina (Cepko, 1999). For example, a series of elegant experiments in the developing spinal cord has unraveled the fine details of the molecular pathways that control both initial neuronal specification and later formation of specific neuronal subtypes, including their final positioning along the rostro-caudal axis of the developing cord, and connectivity to selected peripheral targets (Edlund and Jessell, 1999; Briscoe et al., 2000; Briscoe and Ericson, 2001; Liu et al., 2001; Kania and Jessell, 2003; Lee and Pfaff, 2003; Novitch et al., 2003).

In the mammalian neocortex, the identification of genes that are determinants of neuronal subtypes is complicated by its greater cellular and anatomical complexity compared to many other CNS regions. Here, many different classes of large projection neurons are born from committed progenitors residing in the ventricular and subventricular zone of the developing dorsal telencephalon. These neurons are born in a tightly controlled temporal order, and position themselves, primarily via radial migration, in the developing cortex following an inside-out pattern, to produce the typical six-layered structure seen in the adult mammal (Bayer and Altman, 1991; Rakic, 2002). Each cortical layer contains one or more distinct subtypes of projection neurons that in turn project to different ipsilateral or contralateral cortical, sub-cortical, or sub-cerebral targets. This highly structured anatomical organization is further complicated by the existence of distinct patterns of arealization of the neocortex, and by rostro-caudal and dorso-lateral neuronal gradients (O'Leary and Nakagawa, 2002).

Although decades of elegant studies into cortical development have provided remarkable knowledge about the anatomical and cellular organization of the mammalian cortex, the genetic mechanisms that control its complex neuronal development and diversity are much less known. Over the last ten years, significant progress has been made in identifying some of the genes that control general neuronal specification and areal identity during early cortical development (Ragsdale and Grove, 2001; Bertrand et al., 2002; Rallu et al., 2002). In contrast, while there is some knowledge of laminar-specific markers (Frantz et al., 1994; Nakagawa et al., 1999; Liu et al., 2000; Bulchand et al., 2003), and very limited knowledge of subtype-specific markers (Weimann et al., 1999; Hevner et al., 2001; Bai et al., 2004), the specific molecular programs that direct the differentiation of individual neuronal subtypes have yet to be elucidated (Rallu et al., 2002).

Located primarily in layer V of cortex, corticospinal motor neurons (CSMN) are a critical neuronal subtype. CSMN (“upper motor neuron”) degeneration is a key component of motor neuron degenerative disease, including amyotrophic lateral sclerosis (ALS), and CSMN injury contributes critically to the loss of motor function in spinal cord injury. The anatomical and morphological development of CSMN has been extensively characterized (Jones et al., 1982; Stanfield et al., 1982; Koester and O'Leary, 1993; Terashima, 1995; Joosten and Bar, 1999), but strategies to repair CSMN are limited by a lack of understanding of the molecular controls over CSMN development, including neuron type-specific differentiation, survival, and connectivity.

A few isolated molecules specifically associated with CSMN and related cortical neurons have been identified. These include otx1, a transcription factor expressed in layer V and VI (Frantz et al., 1994; Weimann et al., 1999); er81, a transcription factor of unknown function expressed by multiple neuronal subtypes in layer V, including cortico-cortical projection neurons and CSMN (Hevner et al., 2003); and molecules involved in axonal pathfinding expressed in several types of neurons, including neurons with projections along the corticospinal tract (Coonan et al., 2001; Rolf et al., 2002). Many studies have screened a variety of growth factors for the ability to affect CSMN axonal sprouting or the survival of CSMN during development and repair (Joosten et al., 1996; Junger and Junger, 1998; Giehl, 2001; Bregman et al., 2002), but these investigations have had limited success, at least in part because of a lack of understanding of the molecules and pathways that direct CSMN development, and thus mediate CSMN response to the growth factors investigated.

Thus a greater understanding of the genes that control and or are associated with fate specification, differentiation, survival and connectivity of neuronal subtypes, including CSMN, is needed. Elucidation of such genes will permit a greater understanding of neuronal subtypes and will facilitate directed development and production of neurons useful in treatment of various neurological and neurodegenerative conditions, as well as methods for diagnosis, cell differentiation, screening of candidate therapeutics, etc.

### SUMMARY OF THE INVENTION

Within the vertebrate nervous system, the molecular mechanisms that control the specification and development of most distinct types of neurons are very poorly understood. This is particularly true in the mammalian cerebral cortex, one of the most complex structures of the CNS, in which the presence of many different lineages of neurons and glia contributes to great cellular heterogeneity, and complicates the molecular characterization of single neuronal populations. Here, for the first time, we purified corticospinal motor neurons (CSMN), a clinically important population of cortical projection neurons, callosal projection neurons and corticotectal projection neurons, at distinct stages of development in vivo, and used them to identify genes that are specific and potentially instructive for this neuronal subtype. Using microarrays, we compared the gene expression of purified CSMN and two other pure populations of cortical projection neurons—callosal projection neurons and corticotectal projection neurons. We find genes that are CSMN-specific, as well as genes that are excluded from CSMN and are restricted to other populations of neurons, even within the same cortical layer. Upon further analysis of a subset of largely uncharacterized genes, we find that genes implicated in key developmental processes, from cell fate determination (fez, clim1) to axonal outgrowth (ctip2, netrin-G1) and cell signaling (mu-crystallin, encephalopsin, crim1, igfbp4, pcp4), in addition to one unknown gene that we name csmn1, are all specifically expressed in CSMN. In addition, lmo4 is excluded from CSMN and restricted to other distinct types of neurons. Loss-of function experiments in null-mutant mice for ctip2, one of the newly characterized genes, demonstrate that it plays a critical role in the development of CSMN sub-cerebral axonal projections in vivo, confirming that we identified central genetic determinants of the CSMN population.

Without wishing to be bound by any particular theory, we hypothesize that these CSMN-specific genes that control CSMN specific differentiation may serve in inductive/supportive signaling during the differentiation of immature precursors along the CSMN lineage. Such neuron type-specific molecular controls can be manipulated toward repairing or repopulating CSMN in vivo.

Similarly, we identified a separate set of genes that are specifically expressed in callosal projection neurons. The identification of these genes provides methods and products analogous to those described for CSMN herein.

We also determined that the ctip2 gene is expressed in the striatum specifically in medium spiny projection neurons. This neuron subtype is one of the types that degenerates in Huntington's disease. Therefore, this undestanding can facilitate the development of either neuron transplantation therapy or endogenous stem cell/precursor cell manipulation for this disease and for others in which this subtype of neurons deteriorates. In addition, this result facilitates diagnostic methods and products for identifying medium spiny projection neurons of the striatum.

According to one aspect of the invention, methods for differentiating cells to corticospinal motor neurons (CSMN) are provided. The methods include modulating the activity of one or more CSMN fate specification or end stage differentiation gene products by contacting a population of stem cells, neural and/or neuronal progenitors or precursors with a molecule that modulates expression of one or more CSMN fate specification or end stage differentiation gene products.

According to another aspect of the invention, methods for differentiating cells to corticospinal motor neurons (CSMN). The methods include modulating the activity of one or more CSMN fate specification or end stage differentiation gene products by contacting a population of stem cells, neural and/or neuronal progenitors or precursors with a molecule that is a ligand, activator or repressor of the one or more CSMN fate specification or end stage differentiation gene products.

According to another aspect of the invention, methods for promoting growth of corticospinal motor neurons (CSMN) axons in situ or in culture. The methods include modulating the activity of one or more CSMN axon guidance/process outgrowth promoting gene products by contacting a population of CSMN with a molecule that modulates expression of one or more CSMN axon guidance/process outgrowth promoting gene products that contribute to axon growth.

According to another aspect of the invention, methods for inhibiting, preventing or reversing degeneration of corticospinal motor neurons (CSMN) axons in situ or in culture, or promoting CSMN survival in situ or in culture. The methods include modulating the activity of one or more CSMN survival gene products by contacting a population of CSMN with a molecule that modulates expression of one or more CSMN survival gene products that contribute to CSMN survival.

Analogous methods as described above are provided to promoting differentiation and/or growth of callosal projection neurons (CPN) and medium spiny projection neurons of the striatum (MSPN).

In certain embodiments of the foregoing methods, the one or more gene products are nucleic acids and/or protein molecules. The one or more gene products preferably is/are the expression product of one or more of the genes listed in Table 2 or Table 3 for CSMN, Table 6 for CPN, and Ctip2 for MSPN. In some preferred embodiments, the one or more gene products is the expression product of one or more of the CSMN fate specification or end stage differentiation genes listed in Table 4, particularly the fez and/or clim1 genes, or the ctip2, encephalopsin, pcp4, mu-crystallin, csmn1, igfb4, crim1 and/or netrin-G1 genes. In other preferred embodiments, the one or more gene products is the expression product of one or more of the CSMN axon guidance/process outgrowth promoting genes listed in Table 4, particularly the netrin-G1 and/or ctip2 genes. In other preferred embodiments, the one or more gene products is the expression product of the one or more of the CSMN survival genes listed in Table 4, particularly the ctip2, igfb4 and/or mu-crystallin genes.

In certain embodiments, the expression of the one or more gene products is increased by expressing exogenous nucleic acid molecules that encode the one or more gene products in the population of stem cells, neural and/or neuronal progenitors or precursors. Preferably the exogenous nucleic acid molecules are recombinantly expressed by one or more expression vectors introduced into the stem cells, neural and/or neuronal progenitors or precursors.

In other embodiments, the expression of the one or more gene products is increased by contacting the population of stem cells, neural and/or neuronal progenitors or precursors with a pharmacological molecule that induces increased expression of the one or more gene products, wherein the pharmacological molecule does not encode the one or more gene products.

In still other embodiments, the expression of the one or more gene products is decreased by contacting the population of stem cells, neural and/or neuronal progenitors or precursors with a molecule that reduces expression of the one or more gene products. Preferably the molecule that reduces expression of the one or more gene products is a siRNA molecule, an antisense molecule, or a repressor molecule.

According to another aspect of the invention, methods of cell transplantation are provided. The methods include differentiating or promoting growth of CSMN, CPN or MSPN according to the foregoing methods, exposing the cell in vitro to cell growth conditions to form an expanded CSMN, CPN or MSPN cell population, and administering an amount of the expanded CSMN, CPN or MSPN population or progeny cells produced therefrom to a patient. Preferably the methods further include selecting or sorting the CSMN, CPN or MSPN by contacting the CSMN, CPN or MSPN with a molecule that binds selectively to a CSMN fate specification or end stage differentiation gene product, a CSMN, CPN or MSPN axon guidance/process outgrowth promoting gene product, or a CSMN, CPN or MSPN survival gene product. In some embodiments, the molecule that binds selectively is an antibody or binding fragment thereof, or is detectably labeled.

In other preferred embodiments, the methods further include expressing a detectable molecule under the control of a promoter of a CSMN, CPN or MSPN specific gene products (e.g., fate specification or end stage differentiation gene product, an axon guidance/process outgrowth promoting gene product, or a survival gene products, and selecting or sorting the neuron based on the expression of the detectable molecule. In some embodiments, the detectable molecule is a fluorescent protein, preferably a green fluorescent protein (GFP or EGFP), or is a protein expressed on the CSMN, CPN or MSPN cell surface.

In the foregoing methods, the patient has or is suspected of having a neurodegenerative condition, preferably ALS or neurodegeneration resulting from aging, a spinal cord injury, multiple sclerosis, Huntington's disease, Alzheimer's Disease, autism spectrum disorders, Rett Syndrome, or agenesis/dysgenesis/degeneration of the corpus callosum.

According to another aspect of the invention, methods for identifying lead compounds for a pharmacological agent useful in the differentiation of stem cells, neural and/or neuronal progenitors or precursors to CSMN, CPN or MSPN are provided. The methods include contacting a population of stem cells, neural and/or neuronal progenitors or precursors with a candidate pharmacological agent under conditions that, in the absence of the candidate pharmacological agent, result in a baseline amount of expression of one or more CSMN, CPN or MSPN-specific gene products; and determining a test amount of expression of the one or more CSMN, CPN or MSPN-specific gene products as a measure of the effect of the pharmacological agent on the expression of the one or more CSMN, CPN or MSPN-specific gene products. A test amount of expression of the one or more CSMN, CPN or MSPN-specific gene products that is greater than the baseline amount indicates that the candidate pharmacological agent is a lead compound for a pharmacological agent that is useful in the differentiation of stem cells, neural and/or neuronal progenitors or precursors to corticospinal motor neurons. Preferably the one or more CSMN, CPN or MSPN-specific gene products are one or more fate specification or end stage differentiation gene products, axon guidance/process outgrowth promoting gene products, or survival gene products.

In some embodiments, the compound is a set of compounds in a library of molecules. Preferably the library is a natural product library, a library generated by combinatorial chemistry or a library of known drug molecules.

In certain embodiments of the foregoing methods, the one or more gene products preferably is the expression product of one or more of the genes listed in Table 2 or Table 3 for CSMN, Table 6 for CPN, and Ctip2 for MSPN. In some preferred embodiments, the one or more gene products is/are the expression product of one or more of the CSMN fate specification or end stage differentiation genes listed in Table 4, particularly the fez and/or clim1 genes, or the ctip2, encephalopsin, pcp4, mu-crystallin, csmn1, igfb4, crim1 and/or netrin-G1 genes. In other preferred embodiments, the one or more gene products is the expression product of one or more of the CSMN axon guidance/process outgrowth promoting genes listed in Table 4, particularly the netrin-G1 and/or ctip2 genes. In other preferred embodiments, the one or more gene products is the expression product of the one or more of the CSMN survival genes listed in Table 4, particularly the ctip2, igfb4 and/or mu-crystallin genes.

According to another aspect of the invention, methods for identifying corticospinal motor neurons (CSMN), callosal projection neurons (CPN) or striatal medium spiny projection neurons (MSPN) in a biological sample are provided. The methods include obtaining a biological sample comprising cells, and analyzing the cells of the biological sample for the presence or expression of one or more CSMN, CPN or MSPN-specific gene products. The presence or expression of the one or more CSMN, CPN or MSPN-specific gene products is indicative of CSMN, CPN or MSPN, respectively, in the biological sample. Alternatively or in addition to using a plurality of gene products, a single gene product and a marker of a particular brain structure or region (e.g., neocortex, striatum) can be used to identify specific types of neurons. In some embodiments, the methods further include comparing the results of the analysis of the biological sample to a control sample.

In certain embodiments of the foregoing methods, the one or more gene products preferably is the expression product of one or more of the genes listed in Table 2 or Table 3 for CSMN, Table 6 for CPN, and Ctip2 for MSPN. In some preferred embodiments, the one or more gene products is the expression product of one or more of the CSMN fate specification or end stage differentiation genes listed in Table 4, particularly the fez and/or clim1 genes, or the ctip2, encephalopsin, pcp4, mu-crystallin, csmn1, igfb4, crim1 and/or netrin-G1 genes. In other preferred embodiments, the one or more gene products is the expression product of one or more of the CSMN axon guidance/process outgrowth promoting genes listed in Table 4, particularly the netrin-G1 and/or ctip2 genes. In other preferred embodiments, the one or more gene products is the expression product of the one or more of the CSMN survival genes listed in Table 4, particularly the ctip2, igfb4 and/or mu-crystallin genes.

In some embodiments, the biological sample is a population of cultured neurons, neural and/or neuronal progenitors or precursors. In other embodiments, the one or more CSMN-specific gene products is one or more nucleic acid molecules, which preferably are analyzed using nucleic acid chip expression analysis. In still other embodiments, the one or more CSMN-specific gene products is one or more polypeptides, which preferably are analyzed by antibody binding or proteomic chip analysis.

According to still another aspect of the invention, methods for identifying corticospinal motor neurons (CSMN) in a biological sample are provided. The methods include obtaining a biological sample comprising cells, and analyzing the cells of the biological sample for the presence or expression of one or more CSMN-excluded gene products. The absence of the one or more CSMN-excluded gene products is indicative of CSMN in the biological sample. The methods also can include comparing the results of the analysis of the biological sample to a control sample. Similaraly, these methods can be applied to identifying callosal projection neurons (CPN) or striatal medium spiny projection neurons (MSPN).

In certain embodiments of the foregoing methods, the one or more gene products is the expression product of the one or more of the CSMN-excluded genes listed in Table 4 or 5, preferably lmo4, lix1 and/or tbr1.

In another aspect of the invention, methods for isolating a substantially pure population of corticospinal motor neurons (CSMN) are provided. The methods include selectively labeling a population of cells comprising CSMN by introducing a detectable marker into the CSMN neurons, and isolating the labeled CSMN from unlabeled cells of the population by dissecting the CSMN neurons from other subtypes of neurons, and by sorting the labeled CSMN neurons to obtain a substantially pure population of the CSMN neurons. Likewise, such methods can be used for isolating callosal projection neurons (CPN) and other projection neuron types that send their axons from one location in the nervous system to another. These include both cortico-“X” projection neurons and others not in the cortex, e.g., nigro-striatal neurons for Parkinson's disease, MSPN, etc.

In some embodiments, the labeling step is retrograde labeling that includes microinjecting the cells in a CNS structure that is a target of CSMN at the developmental stage of the organism at the time the injection is performed. Preferably for CSMN the CNS structure is the pons-midbrain junction for embryos, or the pons or the cervical spinal cord at the C2-3 or C5 level for postnatal organisms.

In othe embodiments, the step of dissecting includes enzymatically digesting tissue containing the isolated labeled CSMN and/or mechanically dissociating the tissue containing the isolated labeled CSMN to form a substantially single-cell suspension. Preferably the detectable marker is a fluorescent molecule, and the method further includes isolating labeled CSMN by fluoresence activated cell sorting. The foregoing methods can also include contacting the single-cell suspension with a RNA preservation reagent, preferably RNAlater®. In such methods, the detectable marker preferably is a fluorescent molecule, and the method further includes isolating single labeled CSMN by fluoresence activated cell sorting. The foregoing methods also can include culturing the isolated CSMN. In preferred embodiments of the foregoing methods, the CSMN are isolated at a predetermined developmental stage.

In still other embodiments, the detectable marker is a detectable gene product under the control of a promoter of a CSMN fate specification or end stage differentiation gene product, a CSMN axon guidance/process outgrowth promoting gene product, or a CSMN survival gene product, and the methods include selecting or sorting the CSMN based on the expression of the detectable molecule. In some embodiments, the detectable gene product is a fluorescent protein, preferably a green fluorescent protein (GFP or EGFP), or a protein expressed on the CSMN cell surface.

According to still another aspect of the invention, methods for isolating a substantially pure population of CSMN neurons are provided. The methods include contacting a population of cells comprising CSMN with a molecule that binds selectively to a CSMN fate specification or end stage differentiation gene product, a CSMN axon guidance/process outgrowth promoting gene product, or a CSMN survival gene product, and isolating the CSMN from unlabeled cells of the population by isolating the CSMN bound to the a molecule that binds selectively to obtain a substantially pure population of the CSMN. Preferably the molecule that binds selectively is an antibody or binding fragment thereof, and/or is detectably labeled. Likewise, such methods can be used for isolating substantially pure populations of callosal projection neurons (CPN) and other projection neuron types that send their axons from one location in the nervous system to another. These include both cortico-“X” projection neurons and others not in the cortex, e.g., nigro-striatal neurons for Parkinson's disease, MSPN, etc.

The invention provides in another aspect substantially pure populations of CSMN neurons or substantially pure cultures of CSMN neurons isolated by the foregoing methods. Methods of cell transplantation also are provided that include obtaining a substantially pure population of CSMN or a substantially pure culture of CSMN as provided above, and administering an amount of the CSMN population, culture or progeny cells produced therefrom to a patient. In some embodiments, the patient has or is suspected of having a neurodegenerative condition, preferably ALS. In other embodiments, the patient has or is suspected of having neurodegeneration resulting from aging, a spinal cord injury, or multiple sclerosis. Likewise, such substantially pure populations can included, and methods can be performed using, callosal projection neurons (CPN) and other projection neuron types that send their axons from one location in the nervous system to another. These include both cortico-“X” projection neurons and others not in the cortex, e.g., nigro-striatal neurons for Parkinson's disease, MSPN, etc.

The invention also provides, in another aspect, methods for identifying a nucleic acid gene product expressed specifically or differentially in subtypes of neurons. The methods include obtaining a substantially pure population of two different kinds of neurons, determining a nucleic acid expression profile of the two different kinds of neurons to obtain first and second nucleic acid expression profiles, and determining one or more differences between the first and second nucleic acid expression profiles. The one or more differences between the first and second nucleic acid expression profiles are indicative of the nucleic acid gene products expressed specifically or differentially in the subtypes of neurons. In some embodiments, the nucleic acid expression profile is a mRNA expression profile. In other embodiments, one of the subtypes is corticospinal motor neurons, preferably the isolated population of CSMN or the isolated culture of CSMN above, callosal projection neurons, or corticotecal projection neurons.

In other embodiments, the nucleic acid expression profile is determined by nucleic acid chip analysis or by polymerase chain reaction (PCR) analysis. Preferably the differences in the nucleic acid expression profiles are determined by pairwise comparison of the nucleic acid expression profiles of the subtypes of neurons.

In preferred embodiments, the populations of neurons each comprise at least about 1000 cells, more preferably at least about 10000 cells.

According to another aspect of the invention, methods for identifying lead compounds for a pharmacological agent useful in supporting the growth and/or survival of corticospinal motor neurons (CSMN) are provided. The methods include contacting a population of CSMN with a candidate pharmacological agent under conditions that, in the absence of the candidate pharmacological agent, result in a baseline amount of growth or survival of the CSMN; and determining a test amount of growth or survival of the CSMN in the presence of the pharmacological agent as a measure of the effect of the pharmacological agent on the growth or survival of the CSMN. A test amount of growth or survival of the CSMN that is greater than the baseline amount indicates that the candidate pharmacological agent is a lead compound for a pharmacological agent that is useful in supporting the growth and/or survival of corticospinal motor neurons.

In some embodiments, the compound is a set of compounds in a library of molecules, preferably a natural product library, a library generated by combinatorial chemistry, or a library of known drug molecules. In other embodiments, the population of CSMN are the isolated population or the isolated culture of CSMN provide above.

According to yet another aspect of the invention, methods for preparing a therapeutic agent are provided. The methods include identifying an agent that selectively or preferentially increases the expression or activity of one or more CSMN-specific gene products, and formulating the agent for administration to a subject in need of such treatment. The methods also can include identifying a compound according to the foregoing methods, and formulating the compound for administration to a subject in need of such treatment. Preferred CSMN-specific gene products include CSMN fate specification or end stage differentiation gene products, CSMN axon guidance/process outgrowth promoting gene products, CSMN survival gene products, and CSMN specific gene products of presently unknown function.

Analogous methods and compositions to those provided herein also are provided for callosal neurons and corticotecal neurons. The methods and compositions have applications in treatment and diagnosis of neurodegenerative disease, including amyotrophic lateral sclerosis and Alzheimer's disease, as well as traumatic CNS injuries. Likewise, methods and compositions to those provided herein also are provided for other projection neuron types that send their axons from one location in the nervous system to another. These include both cortico-“X” projection neurons and others not in the cortex, e.g., nigro-striatal neurons for Parkinson's disease, MSPN, etc.

Similarly, analogous methods and compositions to those provided herein also are provided for corticostriatal projection neurons, nigrostriatal neurons and striatal medium spiny projection neurons. The methods and compositions have applications in treatment and diagnosis of neurodegenerative disease, particularly Huntington's disease (corticostriatal projection neurons and striatal medium spiny projection neurons) and Parkinson's disease (corticostriatal projection neurons and nigrostriatal neurons).

Use of the foregoing compositions, populations of neurons and cultures of neurons in the preparation of medicaments, particularly for treating neurodegenerative diseases also is provided.

These and other aspects of the invention are described in greater detail below.

### BRIEF DESCRIPTION OF THE DRAWINGS

FIG. 1. Population-specific Retrograde Labeling of CSMN, Callosal Neurons, and Corticotectal Neurons During Development in vivo, for FACS Purification.

(A-C) In utero ultrasound-guided microinjection of fluorescent microspheres within the pons of an E17 mouse embryo showing (A) the initial positioning of the glass micropipet (arrowheads), (B) the injection at the pons/midbrain junction (arrow), and (C) the embryo post injection, labeled to indicate orientation of the embryo (horizontal ultrasound section). Scale bars, 500 μm. (D) Dorsal view of a P14 brain retrogradely labeled from the C5 level of the cervical spinal cord, showing distinct labeling of CSMN in the regionally delimited motor cortex. (E-L) Low-magnification fluorescence photomicrographs showing (E-H) CSMN and (I-L) callosal projection neurons (CPN) labeled with green fluorescent microspheres in E18, P3, P6 and P14 neocortex. Scale bars, 100 μm. (M) Sagittal P14 brain section, showing distinct labeling of CSMN (red, arrowheads) and corticotectal projection neurons (green, arrows), labeled independently from the spinal cord and superior colliculus, respectively, in the same mouse. Scale bar, 100 μm. The labels II/III, Va, and Vb indicate these cortical laminae; pia, pial surface; ob, olfactory bulb; cb, cerebellum.

FIG. 2. CSMN FACS-purified Following Selective Retrograde Labeling.

(A,B) Sample FACS-plot of the population of CSMN selected; CSMN are selected as (A) a highly fluorescent population (R2; right peak) and (B) based on size (forward scatter) and surface characteristics (side scatter). CSMN appear as a distinct population of large, fluorescent cells (gated as R1 in B). (C-F) FACS-purification results in a pure neuronal population of labeled CSMN. (C, E) mixed cortical cells before FACS-purification; only a very small percentage of dissociated cells are CSMN, recognized as retrogradely labeled from the spinal cord (arrows). Scale bars, 20 μm. (D, F), FACS-purification of CSMN results in an essentially pure, retrogradely labeled population. Scale bars, 20 μm. (F′) FACS-sorted P14 CSMN fixed in RNAlater before sorting often retain short proximal dendritic and/or axonal processes, despite dissociation and FACS-purification. Scale bar, 10 μm.

FIG. 3. Distinct Subtypes of Projection Neurons Share Common Genetic Determinants at Different Stages of Development.

(A) Global cluster of all genes present, normalized across all the samples. At each stage distinct sub-clusters of genes are expressed in each of the three neuronal subtypes analyzed (CSMN, callosal, corticotectal). Columns represent samples and rows represent genes. (B-D) Zoomed images of expression data from the selected areas in (A), showing a selection of highly correlated and biologically relevant genes that are differentially expressed at E18 (B), P3-P6 (C), and P14 (D). These represent examples of a set of common genes that are expressed in a distinct stage-specific manner and which may control early, intermediate, or late aspects of cortical projection neuron development within each of the projection neuron subtypes analyzed. Z-score scale indicates the number of standard deviations from the mean of each data set.

FIG. 4. A Subset of CSMN-specific Genes from Microarray Analysis, Classified into One of Six Groups Based on Expression Profiles Suggesting Biological Roles during CSMN Development.

A subset of biologically interesting genes is shown, selected from a larger group of differentially expressed genes. Each group is represented by a prototypical expression profile shown at left, with a list of six genes per group, a brief description for each gene, and the Genbank ID number. The genes shown in bold are those selected for further analysis in this study. (A) Genes that are expressed at higher levels in CSMN at all stages of development; (B) genes that are highly expressed in CSMN early in development; (C, D) genes that exhibit increasing levels of expression as CSMN develop; (E) genes that are expressed at higher levels in CSMN compared to the closely related population of corticotectal neurons; and (F) genes that are expressed at high levels in other populations of cortical projection neurons, but not in CSMN, thus serving as negative markers for CSMN. CPN, callosal projection neurons; CTPN, corticotectal projection neurons. Graphic gene expression profiles are shown for other genes in FIG. 5, FIG. 9 or FIG. 10.

FIG. 5. Genes Identified from the Microarray Analysis are Specifically Expressed in CSMN.

(A-P) In situ hybridization in coronal (A,C,E-P) or sagittal (B,D) sections of cortex, showing specific expression of all fourteen genes selected in the morphologically distinct population of CSMN (insets, enlarged from boxed areas; small arrows) in layer V. Red arrows indicate the limit of gene expression in the medio-lateral (A,C,E) and rostro-caudal (B,D) axes. Black arrows in B and D indicate sensorimotor cortex, where diap3 and igfbp4 are expressed; arrowheads indicate visual cortex where diap3 and igbp4 expression was not detected. Ages are: P0 (pcp4), P3 (CTIP2, cadherin 13, s1000a10); P6 (crim1, clim1); P14 (diap3, igfbp4, fez, encephalopsin, mu-crystallin, netrin-G1, csmn1, cadherin 22). (B′,D′-P′) Temporal profiles of gene expression from microarray analysis of each selected gene in corticospinal motor neurons (CSMN, ▴) and callosal projection neurons (CPN, ▪). Bars indicate standard errors of the mean. Expression in corticotectal neurons (CTPN, ♦) closely resembles that in CSMN (data not shown), with the exception of a restricted set of genes that discriminate between these closely related projection neuron populations (e.g. diap3, igfbp4, and crim1). Scale bars: (A-P) 100 μm, (A-P inset) 20 μm.

FIG. 6. CTIP2 is Expressed in CSMN and Sub-cerebral Projection Neurons of Layer V but not in Callosal Neurons.

(A) Low-magnification photomicrograph of a sagittal mouse brain section at P6, showing dense labeling of large projection neurons in layer V with anti-CTIP2 antibody (arrows). (B) Same section and as in A, showing FluoroGold labeling of sub-cerebral projection neurons in layer V. (C) Merge image of A and B, showing CTIP2 expression in sub-cerebral projection neurons. Arrows indicate the same positions in all images. Scale bars in A-C, 100 μm. (D-G) CTIP2 is expressed in CSMN. (D) Low-magnification photomicrograph of a coronal section of cortex at P6, showing high levels of CTIP2 expression in layer V (red), and FluoroGold staining of CSMN in the same layer (green). (E) High magnification FluoroGold labeling of CSMN in the boxed area of H. (F) CTIP2 expression in the boxed area in H. (G) merged image of E and F, showing CTIP2 expression in all CSMN. (H) Low-magnification photomicrograph of a coronal section of cortex at P6, showing high levels of CTIP2 expression in layer V (red), and FluoroGold labeled sub-cerebral projection neurons in layer V (green). (I) High magnification image of FluoroGold labeling of sub-cerebral projection neurons in the boxed area of H. (J) CTIP2 expression in the same boxed area in H. (K) Merged image of I and J, showing CTIP2 expression in essentially all sub-cerebral projection neurons. (L) Low-magnification photomicrograph of a coronal section of cortex at P6, showing high levels of CTIP2 expression in layer V (red), and FluoroGold labeled callosal neurons in more superficial layer II/III and deeper layer Vb (CPN; green). (M) High magnification FluoroGold labeling of callosal neurons, and (N) CTIP2 expression in the boxed area in L. (O) Merged image of M and N, showing exclusion of CTIP2 from callosal neurons. (D, H, L) scale bars, 50 μm. (E-G; I-K; M-O) scale bars, 10 μm.

FIG. 7. Developmental Expression of CTIP2 in Neocortex is Restricted to Developing CSMN and other Subcerebral Projection Neurons.

(A-E) Low-magnification photomicrographs of immunocytochemical analysis of CTIP2 expression in the developing brain. (A) At E12, no expression of CTIP2 is detected in the preplate (PP); the arrow indicate a small cluster of CTIP2 expressing cells ventro-lateral to the ganglionic eminence (GE). (B) At E14, CTIP2 is expressed at high levels (arrows) in the developing cortical plate (CP) and developing striatum (asterisk), but not in the ventricular zone or overlying subventricular zone (dashed line near ventricle, LV). (C) At E16, CTIP2 is expressed in the early developing neurons of deep cortical layers (arrows) and in the striatum (asterisks). (D) Expression is maintained at high levels in layer V of cortex and striatum at P3. (E) Low-magnification photomicrograph of a sagittal section at P6, showing high level expression of CTIP2 in layer V of neocortex along the rostral to caudal axis, and in the striatum (asterisk), hippocampus (hp), and olfactory bulb (ob). Scale bars for A-E, 100 μm. Dotted lines indicate the pial surface (Pia), the position of the corpus callosum (cc), and the ventricular margin (LV).

FIG. 8. Ctip2−/− Mice Display Defects in Sub-cerebral Axon Extension and Fasciculation in the Internal Capsule.

(A-C) Low-magnification photomicrographs of coronal sections of wild type brains (+/+) at P0, and (D-F) matched sections from ctip2 null mutant brains (−/−). (A) Wild type brain section, stained with cresyl violet, showing the typical axonal fascicles of the internal capsule (arrows), and corpus callosum (cc). (D) Matched section from a ctip2 null mutant brain (−/−), demonstrating the striking absence of these internal capsule fascicles (arrows), while other fiber tracts, including the corpus callosum (cc), appear normal. L1-expressing axons in the internal capsule of P0 wild type mice (B, C; arrows), are highly fasciculated and tightly bundled, compared to internal capsule axons of ctip2−/− mice (E, F; arrows), which show distinct lack of fasciculation and striking disorganization of these sub-cortical projection axons. This abnormality of axon elongation and fasciculation is evident through the entire rostro-caudal extent of the internal capsule, shown here at both rostral (B, E) and caudal (C, F) locations. (G, J) Low magnification L1-labeled (green) sagittal sections, showing (G) normal fasciculated axons of the internal capsule in wild type controls, compared with the striking abnormal non-fasciculated L1-expressing axons in ctip2−/− mice (J); DAPI nuclear staining (blue). (H,K) High magnification images from the boxed areas in (G) and (J), respectively, reveals the fine details of the non-fasciculated ctip2 null mutant axons (K; arrows) compared to large fascicles in wild type controls (H; arrows); arrowheads in (K) indicate some highly disorganized axonal projections deviating from their normal path in the ctip2 null mutant mice (−/−). Antegrograde DiI-tracing of axons through the internal capsule of E18 wild type (I) and matched ctip2−/− mice (L), showing typical non-fasciculated and disorganized axons in mice lacking ctip2. Many of the disorganized axons in the ctip2 −/− mice possess what appear to be abnormal, bulbous varicosities suggestive of dysmorphic growth cones (arrows in L). (B,C,E,F,G,J), scale bars at 100 μm; (H,K), scale bars at 50 μm; (I,L), scale bars at 10 μm. ctx, cortex; cc, corpus callosum; ic, internal capsule; str, striatum; hp, hippocampus.

FIG. 9. Additional Microarray Gene Expression Profiles for Genes in Corticospinal Motor Neurons Callosal Projection Neurons and Corticotectal Projection Neurons.

CSMN (▴), corticospinal motor neurons; CPN (▪), callosal projection neurons; CTPN (♦), corticotectal projection neurons.

FIG. 10. Microarray Gene Expression Profiles for all other Genes described in FIG. 4, but not included in FIG. 4 or 5.

CSMN (▴), corticospinal motor neurons; CPN (▪), callosal projection neurons; CTPN (♦), corticotectal projection neurons.

FIG. 11. LMO4 is Expressed in Callosal Neurons, but not CSMN.

(A-D) Fluorescence photomicrographs showing exclusion of LMO4 from CSMN in layer V of cortex. (A) Low magnification image of a coronal section of cortex at P6, showing no co-localization of LMO4 (red) with FluoroGold labeled CSMN (green). (B) High magnification image of FluoroGold labeling of CSMN in layer V, and (C) LMO4 expression, in the boxed area in A. (D) Merged image of B and C, showing exclusion of LMO4 from CSMN. (E-L) Fluorescence photomicrographs showing expression of LMO4 in callosal neurons (CPN) of layer II/III (E-H) and layer V (I-L). (E) Low magnification image of a coronal section of cortex at P6, showing broad co-localization of LMO4 (red) with FluoroGold labeled callosal neurons (CPN; green). (F) High magnification image of FluoroGold labeling of callosal neurons in layer II/III of cortex, and (G) LMO4 expression, in the boxed area in E. (H) Merged image of F and G, showing LMO4 expression in essentially all callosal neurons. (I-L) Similarly, LMO4 (K) is expressed in essentially all FluoroGold labeled CPN (J) in layer V of cortex, as shown at low magnification in I, and in the merged image in L. (A, E, I) scale bars, 50 μm. (B-D), (F-H), (J-L) scale bars, 10 μm.

FIG. 12. CTIP2 is Expressed at Low Levels in Deep Layer Corticothalamic Projection Neurons and some Neocortical GABAergic Interneurons, but it is not Expressed in Callosal Neurons, even in the same Layers.

(A) Low-magnification photomicrograph of a coronal section of cortex at P6, showing high levels of CTIP2 expression in layer Va (red). (B,E) FluoroGold labeling of callosal neurons (CPN) and (C,F) CTIP2 expression in the boxed areas in A, in layers II/III and V, respectively. (D) Merged image of B and C; (G) merged image of E and F, showing exclusion of CTIP2 from callosal neurons. (H) Low-magnification photomicrograph of a coronal section of cortex at P6, showing high levels of CTIP2 expression in layer Va (red). (I) FluoroGold labeling of corticothalamic projection neurons (I) and (J) CTIP2 expression in the boxed area in H. (K) Merged image of I and J, showing expression of CTIP2 in essentially all corticothalamic neurons. (L) Low-magnification photomicrograph of a coronal section of cortex at P6, showing high levels of CTIP2 expression in layer Va (red), and GABA expression (green). (M,P) GABAergic interneurons (arrows) and (N,Q) CTIP2 expression in the boxed areas in L, in layers II/III and V, respectively. (O) Merged image of M and N; (R) merged image of P and Q, showing expression of CTIP2 in GABAergic interneurons in superficial upper and deep layers of cortex. (A,H,L) scale bars, 50 μm. (B-D), (E-G), (I-K), (M-O), (P-R) scale bars, 10 μm.

FIG. 13. CSMN in Ctip2^{-/-} Mice Display Pathfinding Defects and Fail to Extend to the Spinal Cord.

(A and E) Schematic representations of sagittal views of the brain and proximal spinal cord in wild-type and Clip2^{-/-} mice, respectively, showing the location of CSMN somas in the cortex (triangles) and their axonal projections toward the spinal cord (lines). (B-D and F-H) Photomicrographs of boxed areas in (A) and (E), respectively. (B and F) Axonal projections by subcerebral projection neurons showing that (B) P0 wild-type axons are organized in typical axon fascicles (arrows), but (F) matched P0 Ctip2^{-/-} null mutant axons are very disorganized, nonfasciculated (arrow), and display axonal projections that deviate from the normal pathway and extend to ectopic targets (arrowhead). (C and G) The same axonal fibers as (B) and (F), at a more caudal location. (C) Wild-type axons are highly organized in tight bundles of fibers progressing unidirectionally toward the pons (arrow), while (G) Ctip2^{-/-} axons are strikingly reduced in numbers with many individual fibers extending to ectopic sites (arrowheads). (D and H) Photomicrographic montages demonstrating (D) that P0 wild-type axons are abundant through the pons (arrows) and have already reached the pyramidal decussation entering the spinal cord (arrowhead). (H) A much smaller number of axons in Ctip2^{-/-} mice enters the pons (arrows) and no axons extend into the medulla or reach the pyramidal decussation. Scale bars, 100 μm.

FIG. 14. Heterozygous Ctip₂^{+/-} Mice Fail to Correctly Prune Sub-cerebral Projections.

(A and D) FG-labeled layer VCSMNin sensorimotor cortex (asterisks) and lateral sensory cortex (boxes) in (A) wild-type and (D) Ctip2^{+/-} heterozygous mice. (B) Higher-magnification image of the area boxed in (A), showing the typical small number of residual CSMN in lateral sensory cortex of 3-week-old wild-type mice. (E) Higher-magnification image of the area boxed in (D), showing the marked increase in the number of residual CSMN in littermate 3-week-old Ctip2^{+/-} heterozygous mice, suggesting that reduced levels of CTIP2 limit the ability of subcerebral projection neurons to properly prune ectopic connections to the spinal cord. (C and F) Camera lucida drawings of (B) and (E), respectively. (G) At 3 weeks of age, Ctip2^{+/-} mice (right bar in each set) retain more than double the number of CSMN in lateral sensory cortex compared to controls (left bar in each set); at 3 weeks, p=0.0002; at 10 weeks, p=0.14. Neuron counts are shown as the mean±SEM of the number of CSMN in every sixth section of lateral sensory cortex of both hemispheres.

FIG. 15. Genes Identified from the Microarray Analysis are Expressed in DiI Retrogradely labeled CSMN in Layer V.

(A,C,E,G) Low-magnification photomicrographs of in situ hybridization analysis of 4 selected genes in coronal sections of P14 cortex. (B,D,F,H and insets) Higher magnification images of the boxed areas in A,C,E,G, showing specific co-localization of each transcript (purple in situ signal) with DiI labeled CSMN (brown photoconversion precipitate). Scale bars: (A,C,E,G) 100 μm, (B,D,F,H) 10 μm, (B′,D′,F′,H′) 5 μm.

### DETAILED DESCRIPTION OF THE INVENTION

Gene expression studies to detect transcripts present in only selected neocortical neurons or expressed at low levels are typically complicated by the cellular heterogeneity of the neocortex (Geschwind, 2000; Lockhart and Barlow, 2001; Luo and Geschwind, 2001; Griffin et al., 2003). Analysis of neuronal subtype-specific genes in cortex is also fundamentally limited by the substantial lack of antigenic markers by which to discriminate among different neuronal subtypes.

We overcame these issues by purifying corticospinal motor neurons (CSMN) and two closely inter-related and anatomically overlapping neuronal subtypes (callosal projection neurons and corticotectal projection neurons) from the murine neocortex at four distinct stages of development (E18, P3, P6, and P14), using fluorescence activated cell sorting (FACS). These stages span critical events of CSMN specification, morphologic maturation, and connectivity. Using microarrays, we compared the gene expression of purified CSMN and these two other pure subtypes. We find genes that are CSMN-specific, as well as genes that are excluded from CSMN and are restricted to other populations of neurons, even within the same cortical layer. Confirmatory analysis of CSMN-specific gene expression for selected candidate genes of particular mechanistic interest, and functional analysis in mutant mice in vivo, further indicate that these genes represent part of a program of novel genetic determinants of the CSMN population.

As used herein, a “CSMN-specific” gene is a gene that is expressed specifically (alone or in combination with other genes) in CSMN and other subcerebral neurons, but not in other cortical neuron subtypes, or is expressed preferentially (alone or in combination with other genes) in CSMN and other subcerebral neurons as compared to other cortical neuron subtypes. The specific or preferential expression may be development stage related, i.e., manifested a one or more developmental stages of CSMN, and not at others. Examples of developmental stage CSMN-specific gene expression are provided in the Examples. Preferential expression includes expression (alone or in combination with other genes) in CSMN at a statistically significantly higher level than in non-CSMN neurons. As described herein, other genes are excluded from expression in CSMN. These “CSMN-excluded” genes are those gene that are expressed (alone or in combination with other genes) in non-CSMN neuron subtypes, such as callosal projection neurons and/or corticotectal projection neurons, but not in CSMN or expressed (alone or in combination with other genes) in CSMN at a level that is statistically significantly lower than in non-CSMN neurons. Due to the nature of gene expression in neuronal subtype determination, the genes that are described herein may be utilized alone or in combination with other genes (including genes not specifically reported here) in the various methods and compositions of the invention.

CSMN-specific gene(s) may be CSMN determinative gene(s), i.e., those gene(s) that, when expressed, contribute to the CSMN phenotype. Several subtypes of CSMN determinative genes have been identified herein. These include, but are not limited to CSMN fate specification or end stage differentiation genes, CSMN axon guidance/process outgrowth promoting genes and CSMN survival genes. Other CSMN determinative genes are CSMN specific genes of unknown function.

CSMN fate specification genes provide instructive signals (e.g., early in development from stem cells or CSMN precursors or progenitors to CSMN) to direct immature neuronal precursors toward a CSMN fate. CSMN end stage differentiation genes provide signals that instruct differentiation to the end stage of CSMN differentiation. CSMN axon guidance/process outgrowth promoting genes provide signals for CSMN axon growth (extension) and/or guidance (pathfinding). CSMN survival genes provide signals for CSMN maturation and survival. Analogous sets of determinative genes for callosal and corticotecal neurons also are provided according to the invention.

The CSMN-specific genes provided herein can be used to differentiate cells to corticospinal motor neurons. These methods can be carried out by modulating the expression or activity of one or more CSMN-specific gene products, such as by contacting a population of stem cells, neural and/or neuronal progenitors or precursors with a molecule that is a ligand, activator or repressor of the CSMN-specific gene products, or by modulating the expression of the CSMN-specific gene products. Differentiated cells can be used in cell transplantation for treatment purposes, in screening assays, etc.

The CSMN-specific genes provided herein also can be used to promote growth of corticospinal motor neurons (CSMN) axons in situ or in culture, and/or to inhibit, prevent or reverse degeneration of corticospinal motor neurons, including their axons. These methods can be carried out by modulating the expression or activity of one or more CSMN-specific gene products in CSMN. CSMN treated in accordance with this aspect of the invention can be used in cell transplantation for treatment purposes. CSMN (and stem cells, precursors and progenitors capable of differentiating into CSMN) also can be treated in situ using the activators or expression modulators of CSMN-specific gene products. In certain embodiments, expression is increased by expressing exogenous nucleic acid molecules that encode the one or more gene products in the population of stem cells, neural and/or neuronal progenitors or precursors, preferably using an expression vector. In other embodiments, a population of stem cells, neural and/or neuronal progenitors or precursors is contacted with a pharmacological molecule that induces increased expression of the one or more gene products.

In various methods disclosed herein, it will be advantageous to select or sort treated cells, differentiate cells, mixed populations of cells, or CSMN in various ways. One method to select or sort cells is by contacting the cells with a molecule that binds selectively to a CSMN-specific gene product. Another method to select or sort cells is by contacting the cells with binding agents specific for CSMN-specific gene products. In addition, cells can be labeled via retrograde transport as described below, with the label being used as a means of detecting certain cells in a population. Another method of labeling the cells is by expressing a detectable molecule (e.g., polypeptide such as green fluorescent protein) under the control of a promoter of a CSMN-specific gene.

Analogous methods and compositions to those provided herein for CSMN also are provided for callosal neurons and corticotecal neurons, corticostriatal projection neurons, nigrostriatal neurons and striatal medium spiny projection neurons.

CSMN-specific genes useful in these methods are provided in the Figures and in Tables 2 and 3 for CSMN, Table 6 for CPN, and Ctip2 for MSPN. CSMN-specific genes of particular function are shown in Table 4. The genes without a specific function are CSMN-specific genes of presently unknown function with respect to CSMN. CSMN-excluded genes, which may be of use as negative markers of CSMN, etc., are provided in Table 5.

For determination of neuron subtype specific genes, as described further below, and for other uses, cells isolated based on labeling can be used without further culture. Moreover, other methods for identifying a nucleic acid gene product expressed specifically or differentially in subtypes of neurons can be carried out using two different kinds of neurons. In such methods, substantially pure populations of the two different kinds of neurons are obtained, and nucleic acid expression profile of the two different kinds of neurons are determined to obtain first and second nucleic acid expression profiles. Differences between the first and second nucleic acid expression profiles are indicative of the nucleic acid gene products expressed specifically or differentially in the subtypes of neurons.

The isolation procedures described herein provide a novel method for determining expression profiles of closely related neuron subtypes, particularly in neuronal tissues in which several neuronal subtypes coexist in close proximity. One advantage of these methods is that a large number of cells can be used in expression profiling, in contrast to single-cell expression profiles which have not been particularly robust methods. It is preferred that at least about 1,000 cells be used in the expression profiling methods; more preferably at least about 10,000 cells are used.

The isolation procedures provide substantially pure populations of neurons, and therefore permit the use of these pure populations of neurons (e.g., CSMN) in methods for screening molecules as pharmacological agents useful in supporting the growth and/or survival of CSMN.

After selecting or sorting, cells can optionally be cultured to expand the population of cells (e.g., for cell transplantation), to subject the cells to further differentiation, to use the cells in screening assays, etc. For cell transplantation, an expanded CSMN population or progeny cells produced therefrom are administered an effective amount a patient. Preferred patients are those that have or are suspected of having a neurodegenerative condition, particularly ALS, spinal cord injury, or multiple sclerosis.

Potential targets for therapy in accordance with the invention include neurodegenerative diseases such as amyotrophic lateral sclerosis (ALS or Lou Gehrig's disease), Alzheimer's disease, Huntington's disease, Parkinson's disease, and other disorders characterized by degeneration of the brain and spinal cord, and traumatic injury to the brain or spinal cord that would benefit from repopulation of subsets of neurons.

A “neurodegenerative disorder” is defined herein as a condition in which there is progressive loss of neurons in the nervous system. Most of the chronic neurodegenerative diseases are typified by onset during the middle adult years and lead to rapid degeneration of specific subsets of neurons within the nervous system, ultimately resulting in premature death.

Amyotrophic lateral sclerosis (ALS) is the most commonly diagnosed progressive motor neuron disease. The disease is characterized by degeneration of motor neurons in the cortex, brainstem and spinal cord (Harrison's Principles of Internal Medicine, 1991 McGraw-Hill, Inc., New York; Tandan et al. Ann. Neurol, 18:271-280, 419-431, 1985). Generally, the onset is between the third and sixth decade, typically in the sixth decade; ALS is uniformly fatal. Although some genetic bases have been demonstrated (e.g., mutations in superoxide dismutase gene on chromosome 21; see Rosen et al., Nature 362:59-62, 1993), these genetic abnormalities do not uniformly exist in ALS patients, and thus the full spectrum of causes of the disease is yet unknown.

In ALS motor neurons of the cerebral cortex, brainstem and anterior horns of the spinal cord are affected. The class of neurons affected is highly specific: motor neurons for ocular motility and sphincteric motor neurons of the spinal cord remain unaffected until very late in the disease. Death in ALS is generally due to respiratory failure secondary to profound generalized and diaphragmatic weakness. About 10% of ALS cases are inherited as an autosomal dominant trait with high penetrance after the sixth decade (Mulder et al. Neurology, 36:511-517, 1986; Horton et al. Neurology, 26:460-464, 1976). In almost all instances, sporadic and autosomal dominant familial ALS (FALS) are clinically similar (Mulder et al. Neurology, 36:511-517, 1986; Swerts et al., Genet. Hum, 24:247-255, 1976; Huisquinet et al., Genet. 18:109-115, 1980). As noted, in some but not all FALS pedigrees the disease is caused by defects in the gene on chromosome 21q that encodes the cytosolic protein, Cu/Zn superoxide dismutase (Rosen et al., Nature 362:59-62, 1993). While a single drug has been approved by the F.D.A. for treatment of ALS, its effect is minimal at best; there is no primary therapy for ALS.

Parkinson's disease (paralysis agitans) is a common neurodegenerative disorder that appears in mid to late life. Familial and sporadic cases occur, although familial cases account for only 1-2 percent of the observed cases. Patients frequently have nerve cell loss with reactive gliosis and formation of Lewy bodies in the substantia nigra and locus coeruleus of the brainstem. Similar changes are observed in the nucleus basalis of Meynert and, in the long term, the nerve cell loss may be quite widespread. As a class, the nigrostriatal dopaminergic neurons seem to be most affected. The disorder generally develops asymmetrically with tremors in one hand or leg and progresses into symmetrical loss of voluntary movement. Eventually, the patient becomes incapacitated by rigidity and tremors. In the advanced stages the disease is frequently accompanied by dementia. Diagnosis of both familial and sporadic cases of Parkinson's disease can only be made after the onset of the disease. While there are symptomatic therapies for Parkinson's disease, there is no primary treatment that slows the underlying neurodegeneration in this disease.

Huntington's disease is a progressive disease characterized by a movement disorder and dementia; it is always transmitted as an autosomal dominant trait. Individuals are asymptomatic until the middle adult years, although some patients show symptoms as early as age 15. Once symptoms appear, the disease is characterized by choreoathetotic movements and progressive dementia until death occurs 15-20 years after the onset of symptoms.

Although some of the characteristic mental depression and motor symptoms associated with Huntington's disease may be suppressed using tricyclic antidepressants and dopamine receptor antagonists, respectively, no therapy exists for slowing or preventing of the underlying disease process. Huntington's disease appears to map to a single gene on chromosome 4 that encodes a protein known as “huntingtin”. The huntingtin gene in its mutant form contains pathological expansions of CAG repeats (see U.S. Pat. No. 5,686,288). A genetic test currently exists for the clinical assessment of disease risk in presymptomatic individuals with afflicted relatives but there is no primary therapy for Huntington's disease.

To provide compositions that modulate expression of one or more CSMN-specific gene products (i.e., nucleic acids and/or polypeptides), a CSMN-specific nucleic acid, in one embodiment, is operably linked to a gene expression sequence which directs the expression of the CSMN-specific nucleic acid within a eukaryotic or prokaryotic cell. Expression of callosal-specific or corticotecal-specific genes is modulated in an analogous manner.

The “gene expression sequence” is any regulatory nucleotide sequence, such as a promoter sequence or promoter-enhancer combination, which facilitates the efficient transcription and translation of the CSMN-specific nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney murine leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Preferably, however, the promoter is a CSMN-specific promoter, which directs gene expression in a cell-type-(CSMN) and developmental stage-specific manner. The promoters for the CSMN-specific genes identified herein are preferred.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined CSMN-specific nucleic acid. The gene expression sequences optionally includes enhancer sequences or upstream activator sequences as desired.

The CSMN-specific nucleic acid sequence and the gene expression sequence are said to be “operably linked” when they are covalently linked in such a way as to place the transcription and/or translation of the CSMN-specific coding sequence under the influence or control of the gene expression sequence. If it is desired that the CSMN-specific sequence be translated into a functional protein, two DNA sequences are said to be operably linked if induction of a promoter in the 5′ gene expression sequence results in the transcription of the CSMN-specific sequence and if the nature of the linkage between the two DNA sequences does not (1) result in the introduction of a frame-shift mutation, (2) interfere with the ability of the promoter region to direct the transcription of the CSMN-specific sequence, or (3) interfere with the ability of the corresponding RNA transcript to be translated into a protein. Thus, a gene expression sequence would be operably linked to a CSMN-specific nucleic acid sequence if the gene expression sequence were capable of effecting transcription of that CSMN-specific nucleic acid sequence such that the resulting transcript might be translated into the desired protein or polypeptide.

The CSMN-specific nucleic acid and the CSMN-specific polypeptide (or, analogously, callosal-specific and/or corticotecal-specific gene products) of the invention can be delivered to the eukaryotic or prokaryotic cell alone or in association with a vector. In its broadest sense, a “vector” is any vehicle capable of facilitating: (1) delivery of a CSMN-specific nucleic acid or polypeptide to a target cell or (2) uptake of a CSMN-specific nucleic acid or polypeptide by a target cell. Preferably, the vectors transport the CSMN-specific nucleic acid or polypeptide into the target cell with reduced degradation relative to the extent of degradation that would result in the absence of the vector. Optionally, a “targeting ligand” can be attached to the vector to selectively deliver the vector to a cell which expresses on its surface the cognate receptor (e.g. a receptor, an antigen recognized by an antibody) for the targeting ligand. In this manner, the vector (containing a CSMN-specific nucleic acid or a CSMN-specific polypeptide) can be selectively delivered to a specific cell. In general, the vectors useful in the invention are divided into two classes: biological vectors and chemical/physical vectors. Biological vectors are more useful for delivery/uptake of CSMN-specific nucleic acids to/by a target cell. Chemical/physical vectors are more useful for delivery/uptake of CSMN-specific nucleic acids or CSMN-specific proteins to/by a target cell.

Biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be attached to the nucleic acid sequences of the invention. Viral vectors are a preferred type of biological vector and include, but are not limited to, nucleic acid sequences from the following viruses: retroviruses, such as Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes virus; vaccinia virus; and polio virus. One can readily employ other vectors not named but known in the art.

Preferred viral vectors are based on non-cytopathic eukaryotic viruses in which non-essential genes have been replaced with the gene of interest. Nonpathogenic and non-cytopathic neurotropic virus vectors are preferred, which can be weakened forms of pathogenic neurotropic viruses. Non-cytopathic viruses include retroviruses, the life cycle of which involves reverse transcription of genomic viral RNA into DNA with subsequent proviral integration into host cellular DNA. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual, ” W.H. Freeman C.O., New York (1990) and Murry, E. J. Ed. “Methods in Molecular Biology,” vol. 7, Humana Press, Inc., Clifton, N.J. (1991).

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Expression vectors containing all the necessary elements for expression of CSMN-specific genes are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a CSMN-specific polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as the pcDNA series of vectors (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element.

In addition to the biological vectors, chemical/physical vectors may be used to deliver a CSMN-specific nucleic acid or polypeptide to a target cell and facilitate uptake thereby. As used herein, a “chemical/physical vector” refers to a natural or synthetic molecule, other than those derived from bacteriological or viral sources, capable of delivering the isolated CSMN-specific nucleic acid or polypeptide to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vesicles which are useful as a delivery vector in vivo or in vitro. It has been shown that large unilamellar vesicles (LUV), which range in size from 0.2-4.0μ can encapsulate large macromolecules. RNA, DNA, and intact virions can be encapsulated within the aqueous interior and be delivered to cells in a biologically active form. In order for a liposome to be an efficient nucleic acid transfer vector, one or more of the following characteristics should be present: (1) encapsulation of the nucleic acid of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Ligands which may be useful for targeting a liposome to a particular cell will depend on the particular cell or tissue type. Additionally when the vector encapsulates a nucleic acid, the vector may be coupled to a nuclear targeting peptide, which will direct the CSMN-specific nucleic acid to the nucleus of the host cell.

Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications.

Other exemplary compositions that can be used to facilitate uptake by a target cell of the CSMN-specific nucleic acids include calcium phosphate and other chemical mediators of intracellular transport, microinjection compositions, electroporation and homologous recombination compositions (e.g., for integrating a CSMN-specific nucleic acid into a preselected location within a target cell chromosome).

The invention also embraces so-called expression kits, which allow the artisan to prepare a desired expression vector or vectors. Such expression kits include at least separate portions of the previously discussed CSMN-specific coding sequences. Other components may be added, as desired, as long as the previously mentioned sequences, which are required, are included.

CSMN-specific cDNA sequences can thus be used in expression vectors to transfect host cells and cell lines, be these prokaryotic (e.g., E. coli), or eukaryotic (e.g., neurons, oocytes, COS cells, yeast expression systems and recombinant baculovirus expression in insect cells). Especially useful are mammalian cells such as human, pig, goat, primate, mouse, rat, etc., which can be used for the identification of molecules that regulate the function of CSMN-specific selectively or preferentially (e.g., by screening chemical compound libraries). The cells may be of a wide variety of tissue types, and include primary cells and cell lines. Specific examples include stem cells, neural precursors or progenitors, neuronal precursors or progenitors, neuronal cell lines including PC12 cells, and Xenopus oocytes. As used herein, “neural precursors” and “neural progenitors” include those cells that are still uncommitted to neuronal vs. astroglial or oligodendroglial fate. As used herein, “neuronal precursors” and “neuronal progenitors” are already committed to become neurons of some type. The set of neural precursors and neural progenitors includes neuronal precursors and neuronal progenitors.

The expression vectors can be used in the various therapeutic, diagnostic and screening methods described herein. For example, expression of CSMN-specific gene products may be performed to obtain polypeptide for antibodies or other diagnostic and therapeutic reagents. Expression of CSMN-specific gene products may be used in therapies for neurodegenerative disease and other disorders in which production of CSMN is desirable, e.g., by increasing expression of CSMN-specific gene products in neurons in vitro for eventual transplantation or in vivo increase of CSMN in situ.

Assays can be performed to screen and/or determine whether a molecule has the ability to modulate CSMN-specific gene product activity, and whether the modulation is selective or preferential. As used herein, “modulation” refers to modulating by at least 10% CSMN-specific gene product expression or activity, preferably modulating by at least 25%, and more preferably modulating by at least 40% as measured by any of the methods well known in the art or as provided herein. Exemplary assays of CSMN-specific gene product expression are described below in the Examples. By “selective inhibition” is meant that the compound modulates gene product expression or activity in a CSMN-specific manner, e.g., in CSMN but not significantly in other neurons including closely related neurons, i.e., callosal or coritocotecal neurons. By “preferential modulation” is meant that the compound modulates gene product expression or activity in CSMN by at least about 5% more than gene product expression or activity in other neuron subtypes, such as callosal or coritocotecal neurons. Preferably, the preferential modulation is at least about 10% more for CSMN, more preferably at least about 20% more for CSMN, still more preferably at least about 30% more for CSMN, yet more preferably at least about 40% more for CSMN, and most preferably at least about 50% more for CSMN. Greater differences in modulation of CSMN-specific gene products than non-CSMN-specific gene products is contemplated, from 51% all the way up to about 99%, at which point the inhibition may be considered selective. Molecules may selectively or preferentially modulate CSMN-specific gene products by modulating transcription, translation, or activity of the CSMN-specific gene products.

In screening for modulators of CSMN-specific gene products, including inhibitors and activators (i.e. antagonists and agonists), it is preferred that molecules (e.g., libraries of potential modulators) are tested for modulation of CSMN-specific gene product expression at a variety of developmental stages in stem cells, neural precursors or progenitors, neuronal precursors or progenitors, and/or neurons. These assays can include the assays described in the Examples herein. Such compounds are useful for selectively modulating CSMN-specific gene products in the various stages of development, and may be used combinatorially and/or sequentially to direct CSMN-specific development. For example, stem cells may be treated with one or more modulators in a sequential manner in order to mimic the natural development of CSMN. Other uses will be apparent to one of ordinary skill in the art.

The invention further provides efficient methods of identifying pharmacological agents or lead compounds for agents useful in the treatment of conditions associated with neurodegeneration, particularly those conditions involving degeneration of CSMN neurons, and the compounds and agents so identified. Generally, the screening methods involve assaying for compounds which modulate (inhibit or enhance) the expression or activity of CSMN-specific gene products. Such methods are adaptable to automated, high throughput screening of compounds. Examples of such methods are described in U.S. Pat. No. 5,429,921.

A variety of assays for pharmacological agents are provided, including, labeled in vitro protein binding assays, gene expression assays, etc. For example, protein binding screens are used to rapidly examine the binding of candidate pharmacological agents to a CSMN-specific polypeptide. Gene expression screens examine the modulation of CSMN-specific gene product expression via methods such as those detailed in the Examples. The candidate pharmacological agents can be derived from, for example, combinatorial peptide libraries, combinatorial chemical compound libraries, and natural products libraries. Convenient reagents for such assays are known in the art.

For certain methods, such as CSMN-specific gene expression assays, cells that express a determinable quantity of CSMN-specific gene products are used; the effect of the test molecules on CSMN-specific gene product expression is determined. For other methods, CSMN-specific gene products can be added to an assay mixture as an isolated polypeptide (where binding of a candidate pharmaceutical agent is to be measured) or as a cell or other membrane-encapsulated space which includes a CSMN-specific polypeptide. In the latter assay configuration, the cell or other membrane-encapsulated space can contain the CSMN-specific gene product as a preloaded polypeptide or as a nucleic acid (e.g. a cell transfected with an expression vector containing a nucleic acid that encodes a CSMN-specific polypeptide). In the assays described herein, the CSMN-specific polypeptide can be produced recombinantly, or isolated from biological extracts, but preferably is synthesized in vitro. CSMN-specific polypeptides encompass chimeric proteins comprising a fusion of a CSMN-specific polypeptide with another polypeptide, e.g., a polypeptide capable of providing or enhancing protein-protein binding, or enhancing stability of the CSMN-specific polypeptide under assay conditions. A polypeptide fused to a CSMN-specific polypeptide or fragment thereof may also provide means of readily detecting the fusion protein, e.g., by immunological recognition or by fluorescent labeling.

For the cell-based assay described herein, preferred cell types are neurons. Matched control cells can be used in the assays, e.g., cells that do not express CSMN-specific gene products.

The assay mixture also comprises a candidate pharmacological agent molecule. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500. Candidate agents comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid, the agent typically is a DNA or RNA molecule, although modified nucleic acids having non-natural bonds or subunits are also contemplated. Thus, antisense and siRNA molecules can be tested for inhibition of CSMN-specific gene product expression by these assays and other standard assays of nucleic acid expression, such as gene chips as described here and PCR. Utilizing the cell-based assays described above allows the identification of antisense and siRNA molecules that inhibit function of CSMN-specific gene products.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily modified through conventional chemical, physical, and biochemical means. Further, known pharmacological agents may be subjected to directed or random chemical modifications such as acylation, alkylation, esterification, amidification, etc. to produce structural analogs of the agents.

Candidate agents can be selected randomly or can be based on existing compounds which bind to and/or modulate the function of CSMN-specific gene products, e.g., once identified through screening. The structure of a candidate agent can be changed at one or more positions of the molecule to contain more or fewer chemical moieties or different chemical moieties. The structural changes made to the molecules in creating the libraries of analog modulators can be directed, random, or a combination of both directed and random substitutions and/or additions. One of ordinary skill in the art in the preparation of combinatorial libraries can readily prepare such libraries.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The mixture of the foregoing assay materials is incubated under conditions whereby, but for the presence of the candidate pharmacological agent, a control amount of CSMN-specific gene product expression or activity is obtained. For determining the binding of a candidate pharmaceutical agent to a CSMN-specific gene product, the mixture is incubated under conditions which permit binding. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the level of CSMN-specific gene product expression or activity is detected by any convenient method available to the user. For cell free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximize signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as a gene expression assay as described herein (e.g., using microarrays). For cell free binding assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a CSMN-specific polypeptide or the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

In another embodiment, the invention provides similar assays using CSMN-excluded gene products to identify modulators of CSMN-excluded gene product expression and function. In one particular embodiment, the modulator is an antisense oligonucleotide or siRNA molecule that selectively binds to a nucleic acid molecule, to reduce the expression of the encoded gene product in a cell. An example of this is in CSMN-excluded genes, whereby the use of the antisense oligonucleotide or siRNA molecule reduces the expression of CSMN-excluded genes to exclude differentiation into non-CSMN neurons. Another example is the use of antisense oligonucleotides or siRNA molecules to reduce the expression of CSMN-specific genes at a particular stage of differentiation as found with CSMN neurons in vivo as reported herein. Still another example is the use of the antisense oligonucleotides or siRNA molecules to determine whether the expression of a particular CSMN-specific gene is essential to the CSMN phenotype.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of the gene product of interest. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of the gene of interest is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. Preferably the nucleotide encoding sequence is part of the coding sequence of the gene of interest. The siRNA can be expressed from a vector introduced into cells.

Vectors comprising any of the nucleotide coding sequences of the invention are provided for production of siRNA, preferably vectors that include promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2/V5-GW/lacZ, pENTRiU6, pLenti6-GW/U6-laminshrna and pLenti6/BLOCK-iT-DEST (Invitrogen). These vectors and others are available from commercial suppliers.

It is preferred that the antisense oligonucleotide or siRNA molecule be constructed and arranged so as to bind selectively with the target under physiological conditions, i.e., to hybridize substantially more to the target sequence than to any other sequence in the target cell under physiological conditions. One of skill in the art can easily choose and synthesize any of a number of appropriate antisense or siRNA molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. For siRNA molecules, it is preferred that the molecules be 21-23 nucleotides in length, with a 3′ 2 nucleotide overhang, although shorter and longer molecules and molecules without overhangs are also contemplated as useful in accordance with the invention.

The antisense is targeted, preferably, to sites in which mRNA secondary structure is not expected (see, e.g., Sainio et al., Cell Mol. Neurobiol. 14(5):439-457, 1994) and at which polypeptides are not expected to bind. Other methods for selecting preferred siRNA sequences are known to those of skill in the art (e.g., the “siRNA Selection Program” of the Whitehead Institute for Biomedical Research (2003)).

In one set of embodiments, the antisense oligonucleotides or siRNA molecules of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors, including in situ.

In preferred embodiments, however, the antisense oligonucleotides or siRNA molecules of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, the gene product of interest, together with pharmaceutically acceptable carriers.

Antisense oligonucleotides or siRNA molecules may be administered as part of a pharmaceutical composition. Such a pharmaceutical composition may include the antisense oligonucleotides or siRNA molecules in combination with any standard pharmaceutically acceptable carriers which are known in the art. The compositions should be sterile and contain a therapeutically effective amount of the antisense oligonucleotides or siRNA molecules in a unit of weight or volume suitable for administration to a patient. The term “pharmaceutically acceptable” means a non-toxic material that does not interfere with the effectiveness of the biological activity of the active ingredients. The characteristics of the carrier will depend on the route of administration. Pharmaceutically acceptable carriers include diluents, fillers, salts, buffers, stabilizers, solubilizers, and other materials which are well known in the art.

Agents which bind CSMN-specific gene products also include binding peptides which bind to the CSMN-specific gene products and complexes containing the CSMN-specific gene products. To determine whether a binding peptide binds to CSMN-specific gene products, any known binding assay may be employed. For example, the binding peptide may be immobilized on a surface and then contacted with a labeled CSMN-specific polypeptide. The amount of CSMN-specific polypeptide which interacts with the binding peptide or the amount which does not bind to the binding peptide may then be quantitated to determine whether the binding peptide binds to the CSMN-specific polypeptide. Further, the binding of a CSMN-specific polypeptide and a binding peptide can be compared to determine if the binding peptide binds selectively or preferentially.

The binding peptides include peptides of numerous size and type that bind selectively or preferentially to CSMN-specific polypeptide, and complexes of both CSMN-specific polypeptide and their binding partners. These peptides may be derived from a variety of sources. For example, binding peptides can be identified by screening degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the CSMN-specific polypeptide. This process can be repeated through several cycles of reselection of phage that bind to the CSMN-specific polypeptide. Repeated rounds lead to enrichment of phage bearing particular sequences. DNA sequence analysis can be conducted to identify the sequences of the expressed polypeptides. The minimal linear portion of the sequence that binds to the CSMN-specific polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the CSMN-specific polypeptide. Thus, the CSMN-specific polypeptide of the invention, or a fragment thereof, can be used to screen peptide libraries, including phage display libraries, to identify and select peptide binding partners of the CSMN-specific polypeptides of the invention. Such molecules can be used, as described, for screening assays, for purification protocols, for interfering directly with the functioning of CSMN-specific polypeptide and for other purposes that will be apparent to those of ordinary skill in the art. Peptides may easily be synthesized or produced by recombinant means by those of skill in the art.

The binding peptide agent may also be an antibody or a functionally active antibody fragment. Antibodies are well known to those of ordinary skill in the science of immunology. As used herein, the term “antibody” means not only intact antibody molecules but also fragments of antibody molecules retaining CSMN-specific polypeptide binding ability (antigen binding fragments). Such fragments are also well known in the art and are regularly employed both in vitro and in vivo. In particular, as used herein, the term “antibody” means not only intact immunoglobulin molecules but also well-known active fragments such as F(ab′)₂ and Fab and Fv (including single chain antibodies).

An antibody refers to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding fragment” of an antibody as used herein, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546) which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

An “isolated antibody”, as used herein, is intended to refer to an antibody which is substantially free of other antibodies having different antigenic specificities (e.g., a population of isolated antibodies that specifically binds to a CSMN-specific polypeptide, is substantially free of antibodies that specifically bind antigens other than a CSMN-specific polypeptide). An isolated antibody that specifically binds to an epitope, isoform or variant of a CSMN-specific polypeptide may, however, have cross-reactivity to other related antigens, e.g., from other species (e.g., species homologs). Moreover, an isolated antibody may be substantially free of other cellular material and/or chemicals. As used herein, “specific binding” refers to antibody binding to a predetermined antigen. Typically, the antibody binds a CSMN-specific polypeptide or a closely-related antigen with an affinity that is at least two-fold greater than its affinity for binding to a non-specific antigen (e.g., BSA, casein) or the CSMN-specific polypeptide.

The isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)2 fragment, and a Fv fragment.

As used herein, antibodies also include single chain antibodies (e.g., scFvs). In some embodiments, the single chain antibodies are disulfide-free antibodies having mutations e.g., in disulfide bond forming cysteine residues. The antibodies may be prepared by starting with any of a variety of methods, including administering a CSMN-specific polypeptide, fragments of a CSMN-specific polypeptide, cells expressing the protein or fragments thereof and the like to an animal to induce polyclonal antibodies. Such antibodies or antigen-binding fragments thereof may be used in the preparation of scFvs and disulfide-free variants thereof. The antibodies or antigen-binding fragments thereof may be used for example to modulate the activity of a target protein.

Various forms of the antibody polypeptide or encoding nucleic acid can be administered and delivered to a mammalian cell (e.g., by virus or liposomes, or by any other suitable methods known in the art). The method of delivery can be modified to target certain cells, and in particular, cell surface receptor molecules or antigens present on specific cell types. Methods of targeting cells to deliver nucleic acid constructs, for intracellular expression of the antibodies, are known in the art. The antibody polypeptide sequence can also be delivered into cells by expressing a recombinant protein fused with peptide carrier molecules. These carrier molecules, which are also referred to herein as protein transduction domains (PTDs), and methods for their use, are known in the art. Examples of PTDs, though not intended to be limiting, are tat, antennapedia, and synthetic poly-arginine. These delivery methods are known to those of skill in the art and are described in U.S. Pat. No. 6,080,724, and U.S. Pat. No. 5,783,662, the entire contents of which are hereby incorporated by reference.

The antibodies of the present invention can be polyclonal, monoclonal, or a mixture of polyclonal and monoclonal antibodies. The antibodies can be produced by a variety of techniques well known in the art. Procedures for raising polyclonal antibodies are well known and are disclosed for example in E. Harlow, et. al., editors, Antibodies: A Laboratory Manual (1988), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide, e.g., with a CSMN-specific polypeptide. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. See; Goding (in Monoclonal Antibodies: Principles and Practice, 2d ed., pp. 60-61, Orlando, Fla., Academic Press, 1986).

The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. In particular, mouse strains that have human immunoglobulin genes inserted in the genome (and which cannot produce mouse immunoglobulins) are preferred. Such mice produce fully human immunoglobulin molecules in response to immunization.

The preparations of the invention, such as a composition that modulates expression of one or more CSMN-specific gene products or an activator of one or more CSMN-specific gene products, are administered in effective amounts. An effective amount is that amount of a pharmaceutical preparation that alone, or together with further doses, produces the desired response. In the case of treating a condition characterized by neurodegeneration, the desired response is slowing neurodegeneration or increasing the presence of neurons to a level which is within a normal range. The responses can be monitored by routine methods in the art, such as standard clinical assessments of neurological function and diagnostic methods provided by the invention.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Generally, doses of active compounds would be from about 0.01 ng/kg per day to 1000 mg/kg per day. It is expected that doses ranging from 50 μg-500 mg/kg will be suitable and in one or several administrations per day. Lower doses can result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compound, although fewer doses typically will be given when compounds are prepared as slow release or sustained release medications.

When administered, the pharmaceutical preparations of the invention are applied in pharmaceutically-acceptable amounts and in pharmaceutically-acceptably compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions that modulate expression of one or more CSMN-specific gene products or activators of one or more CSMN-specific gene products useful according to the invention may be combined, optionally, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; and phosphoric acid in a salt.

The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular compound selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intrathecal, intracranial, intramuscular, or infusion.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous preparation of a composition that modulates expression of one or more CSMN-specific gene products or an activator of one or more CSMN-specific gene products, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intrathecal, intramuscular, etc. administrations can be found in Remington's Pharmaceutical Sciences, Mack Publishing Co., Easton, Pa.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. Use of a long-term sustained release implant may be desirable. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The invention will be more fully understood by reference to the following examples. These examples, however, are merely intended to illustrate the embodiments of the invention and are not to be construed to limit the scope of the invention.

### EXAMPLES

### Example 1

### Neuronal Subtype-Specific Genes that Control Corticospinal Motor Neuron Development In Vivo

Experimental Procedures

Neuronal Subtype Labeling

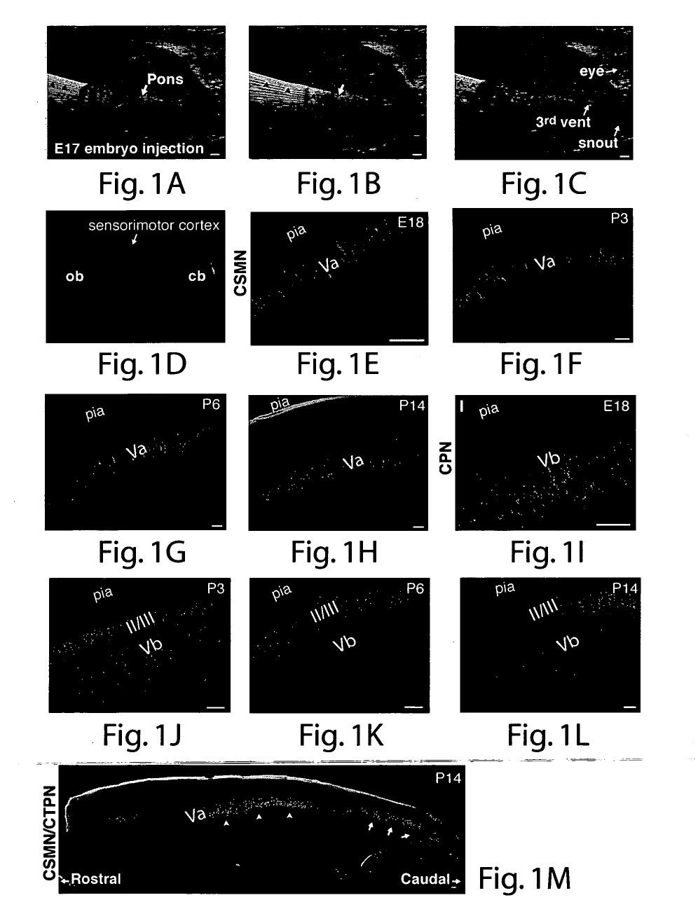

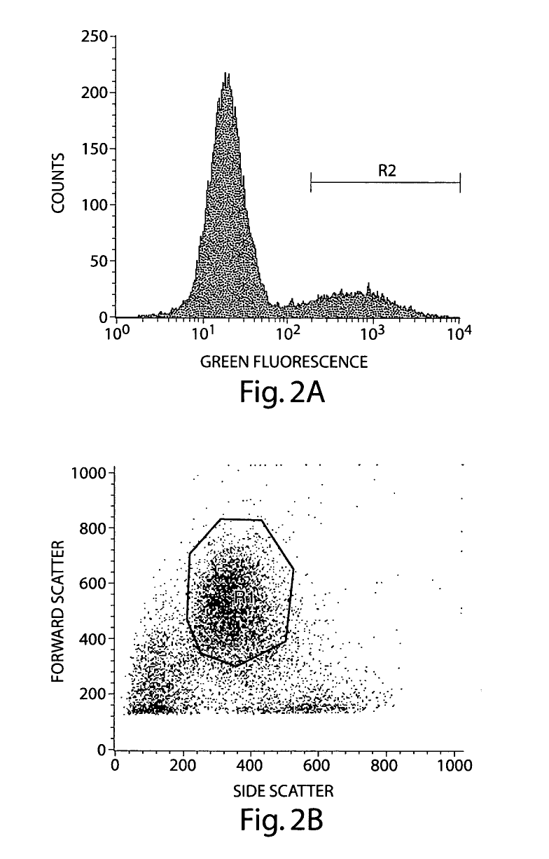

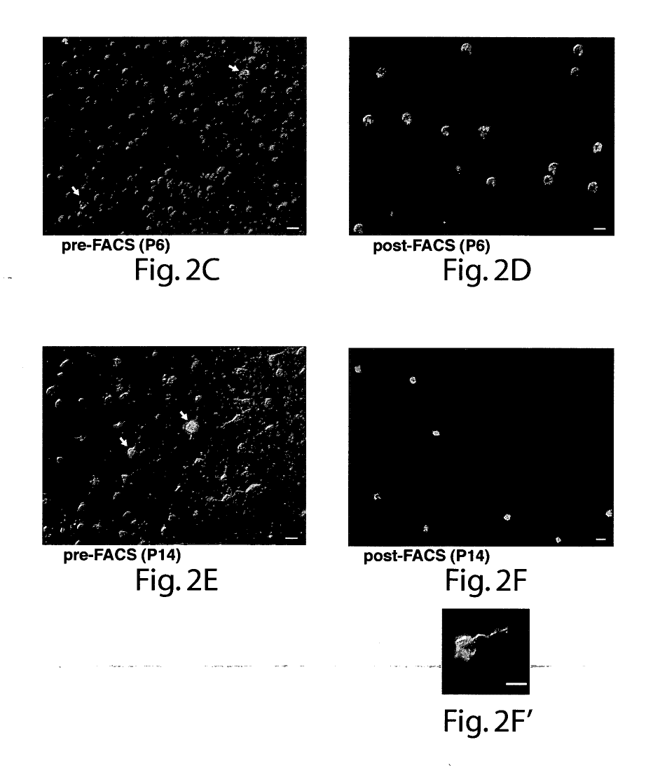

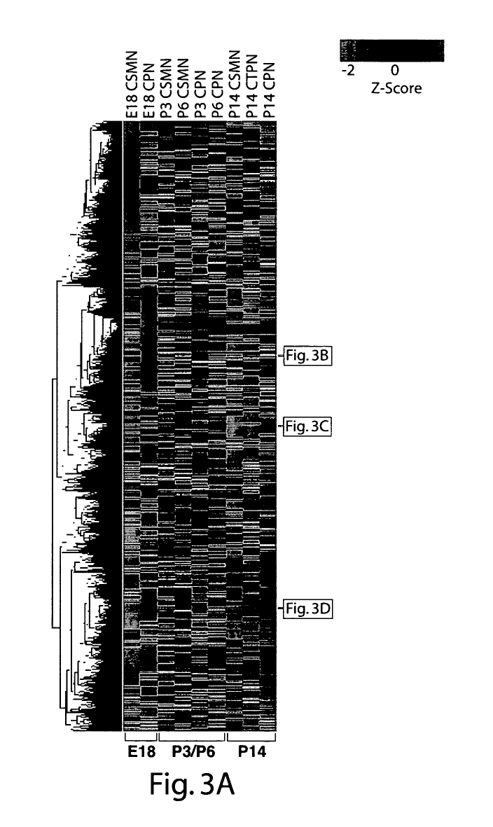

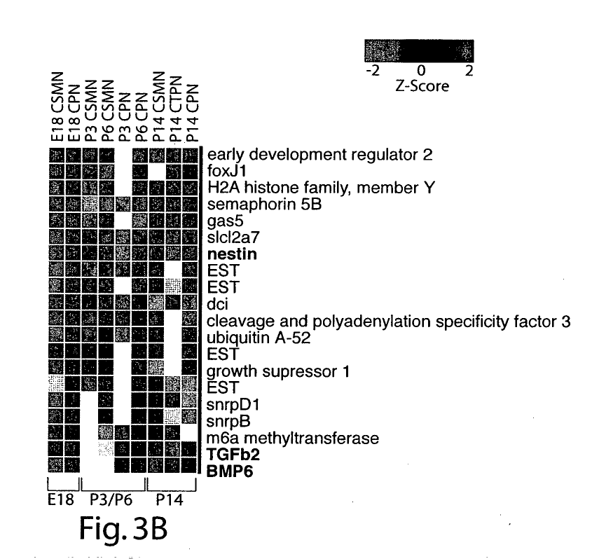

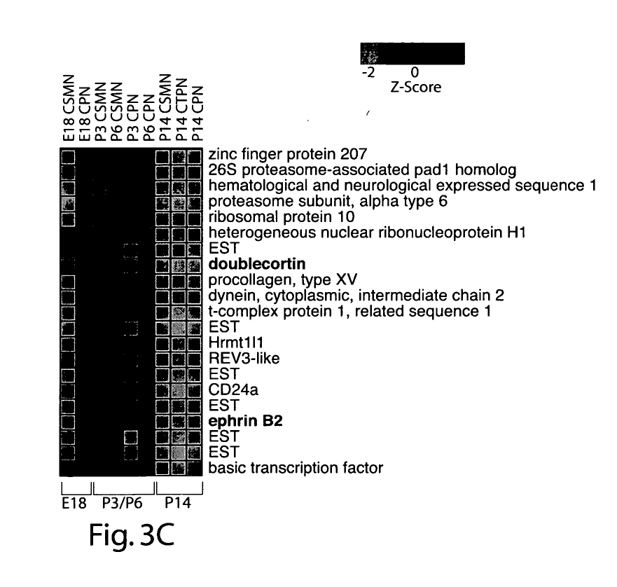

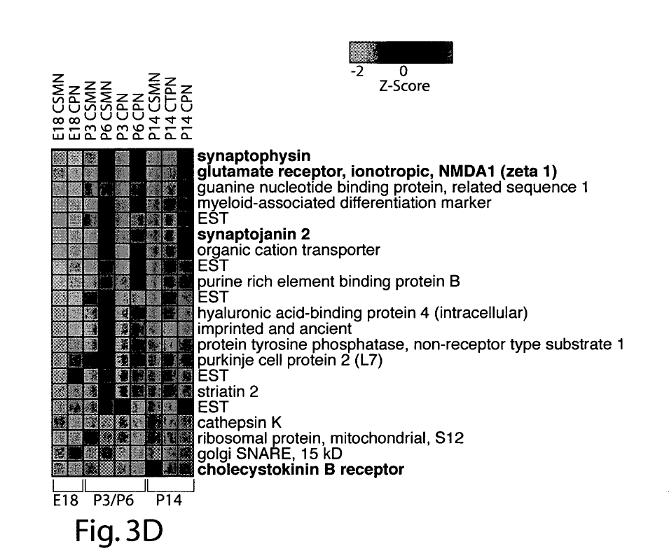

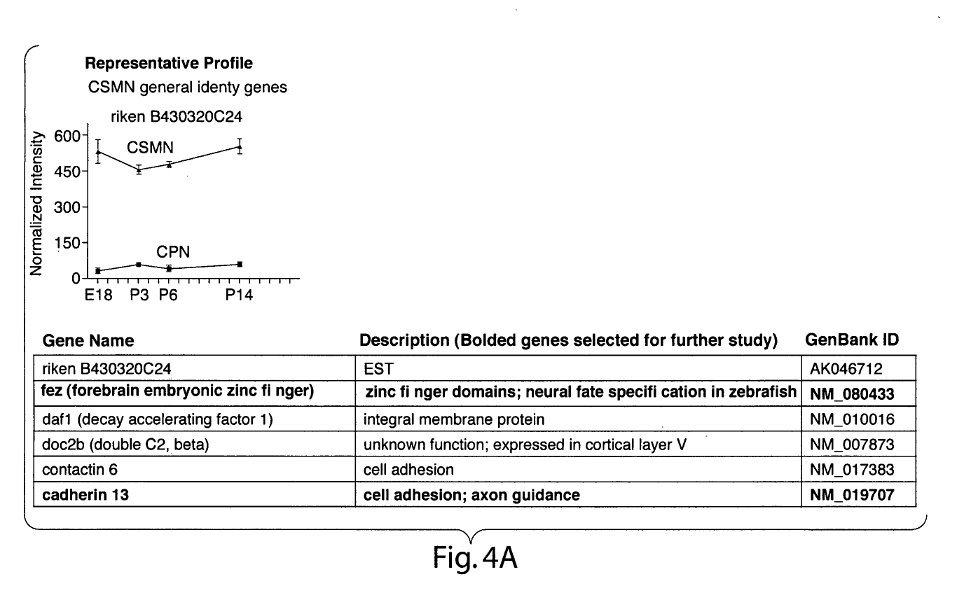

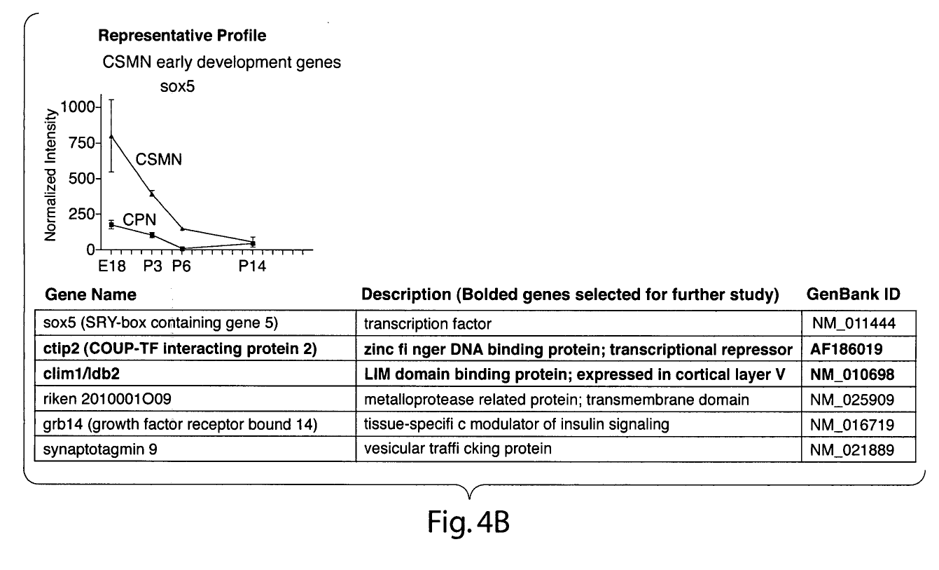

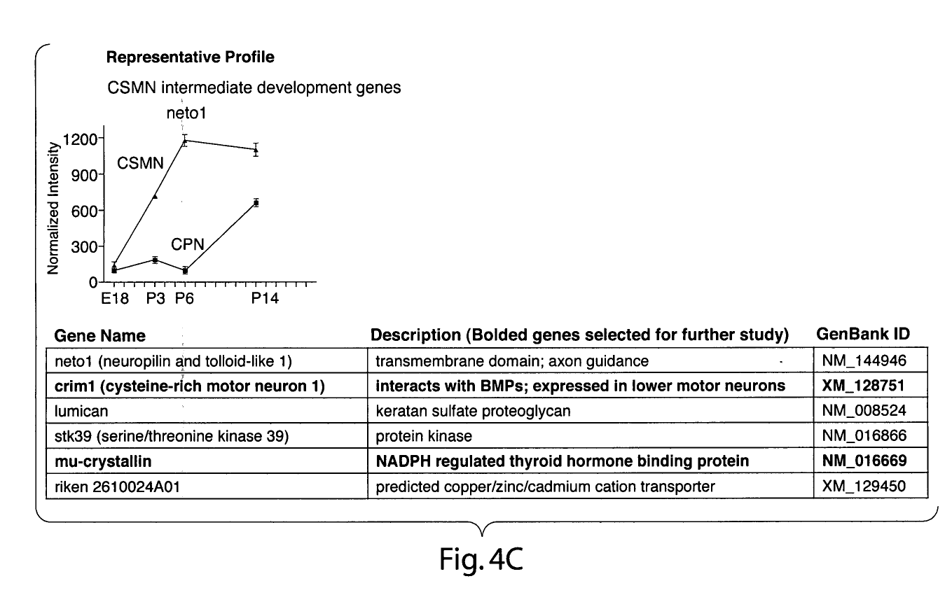

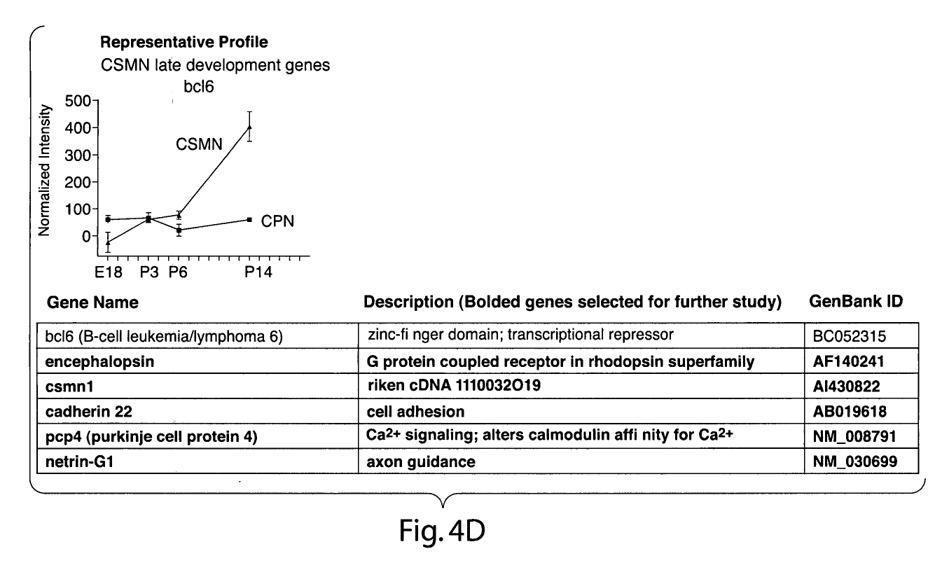

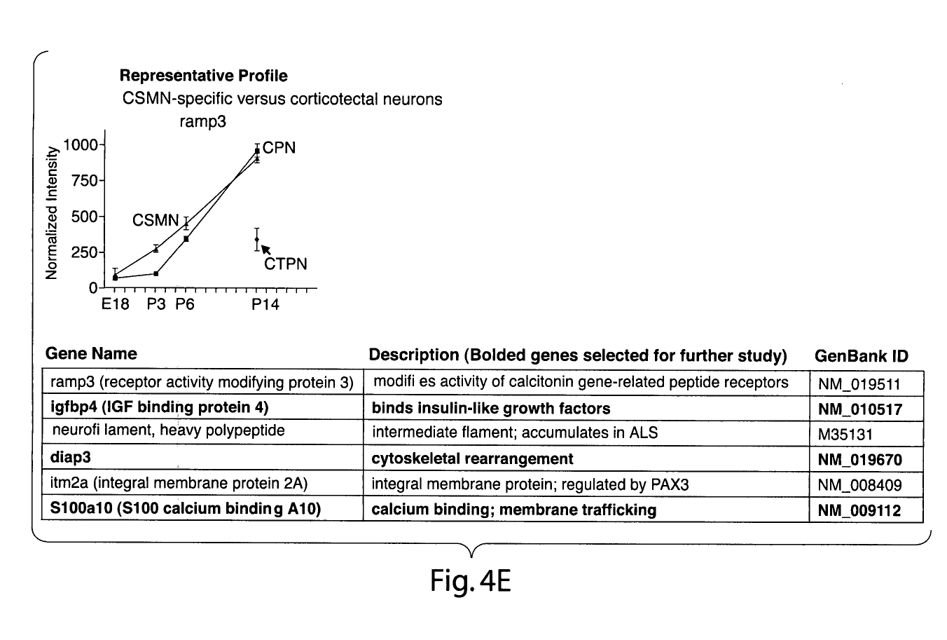

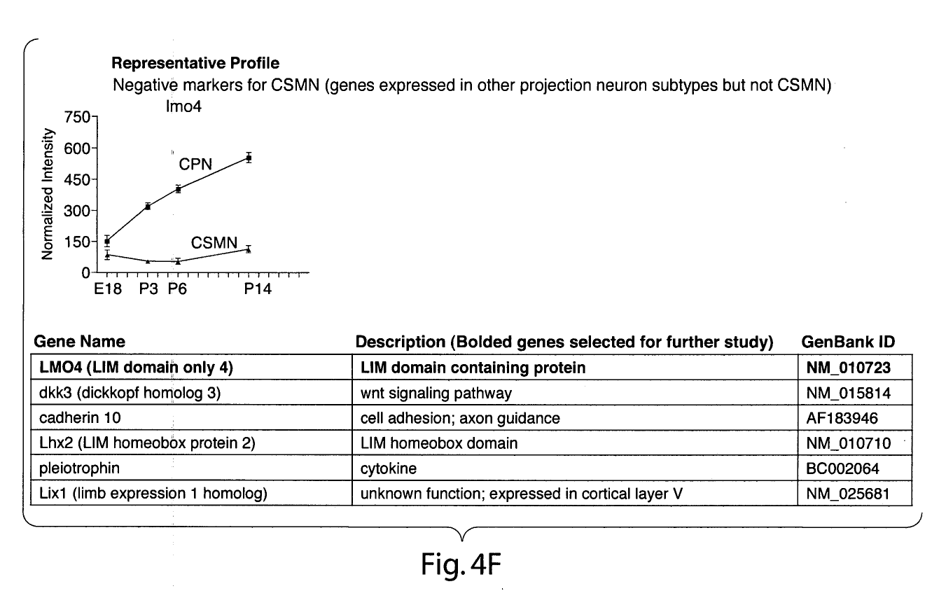

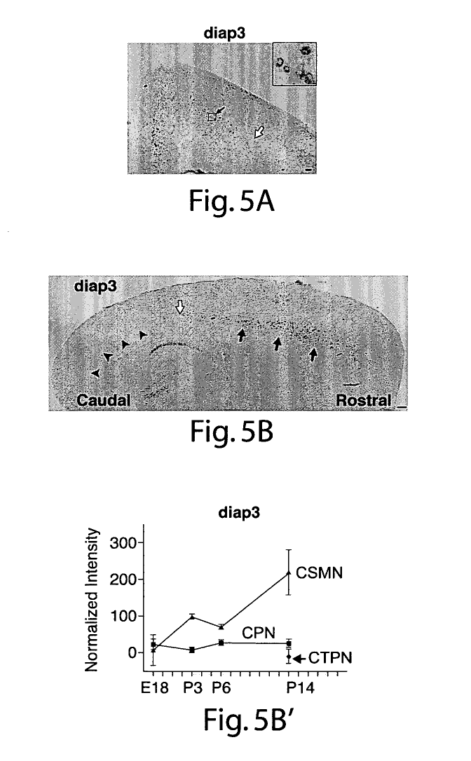

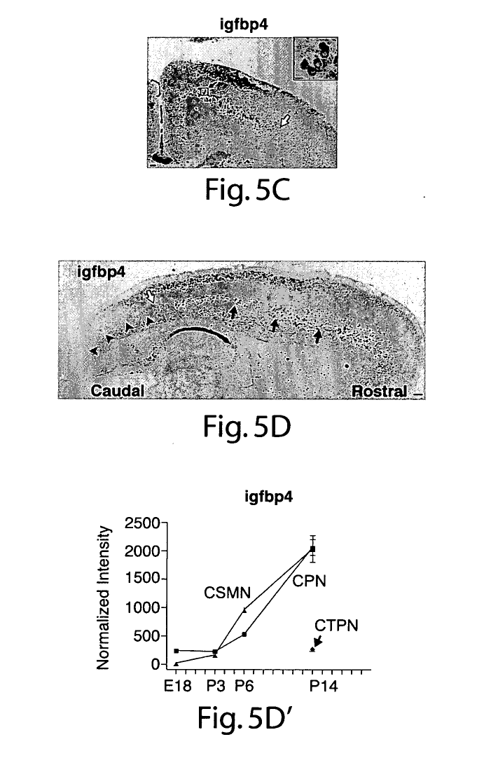

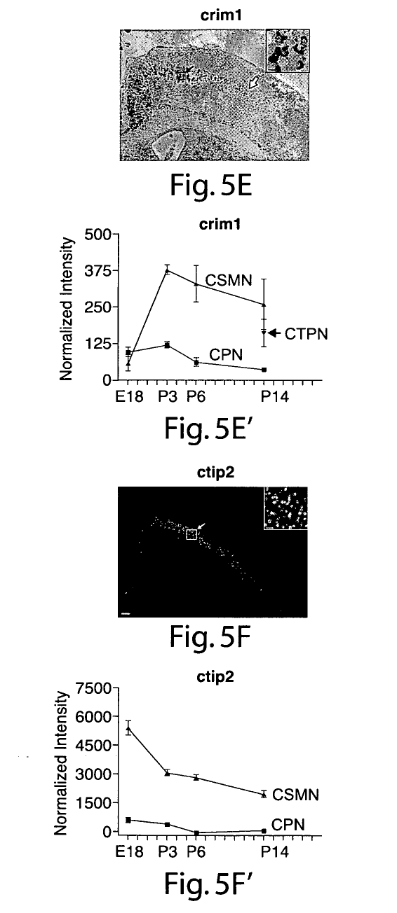

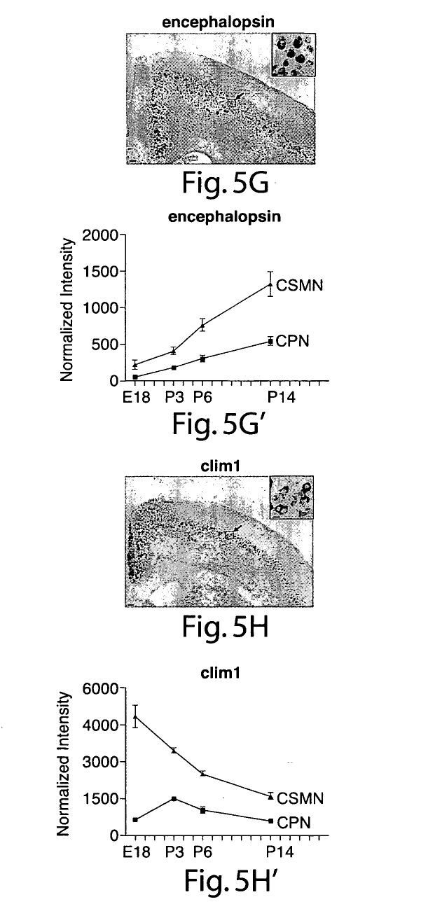

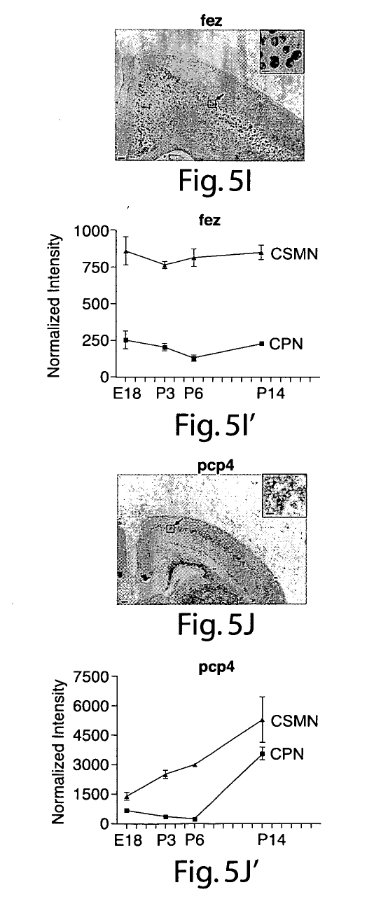

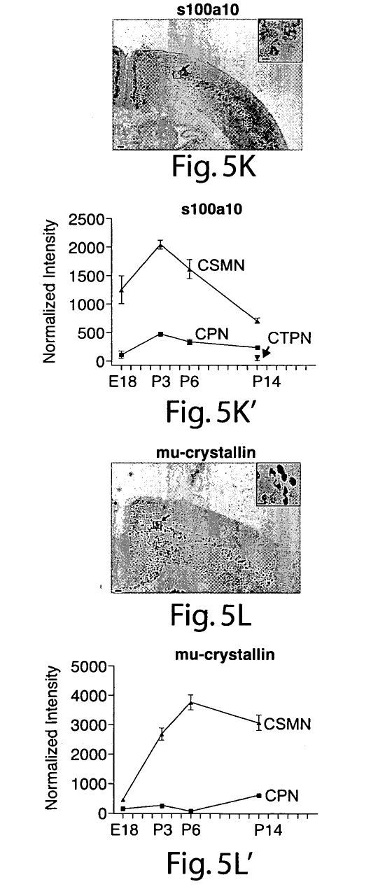

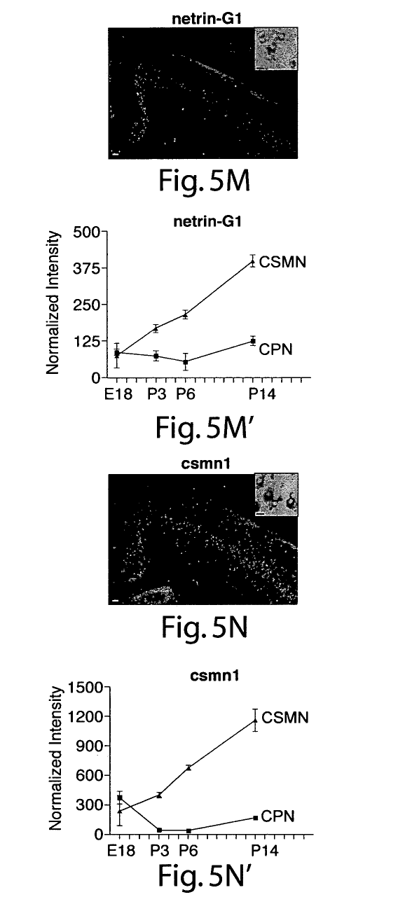

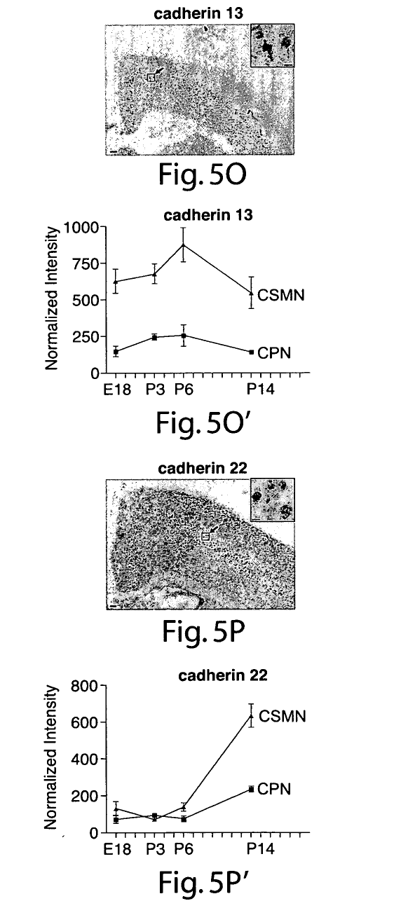

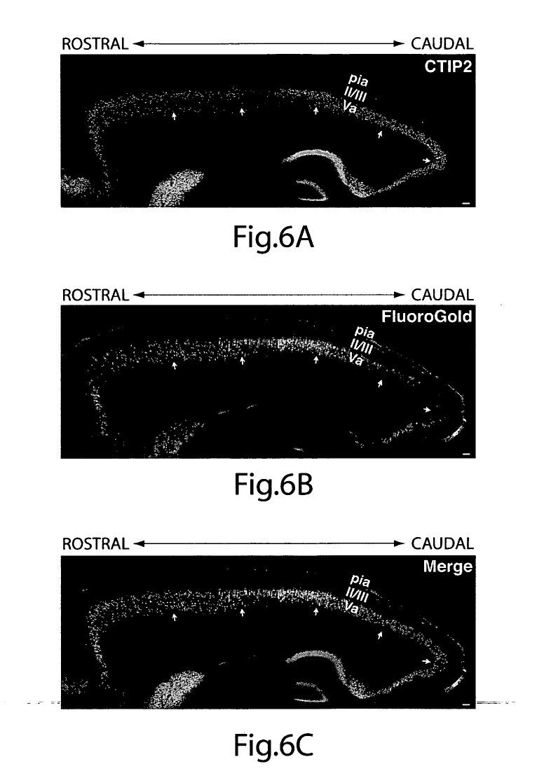

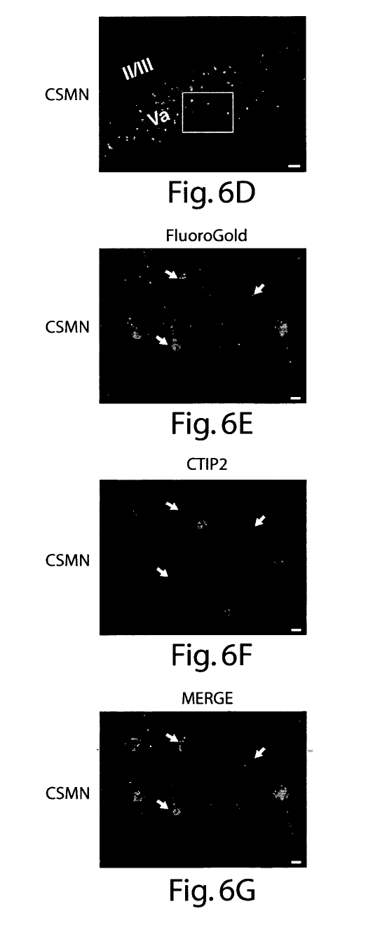

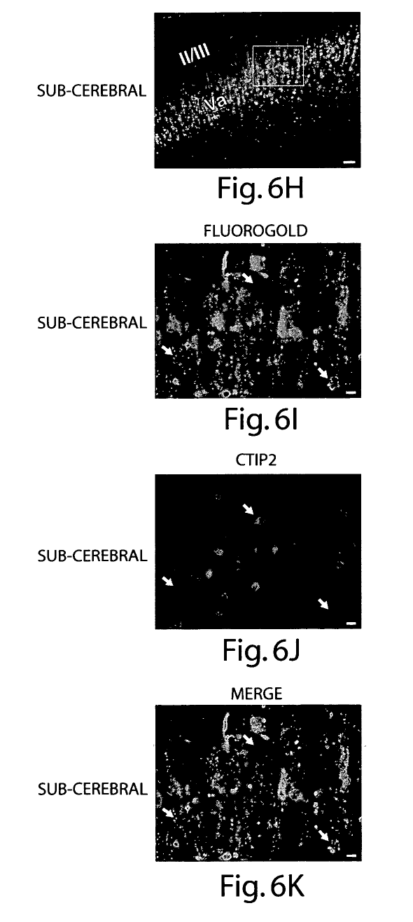

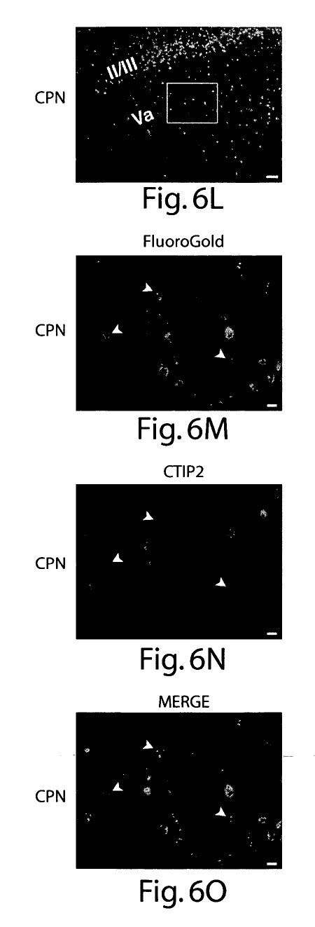

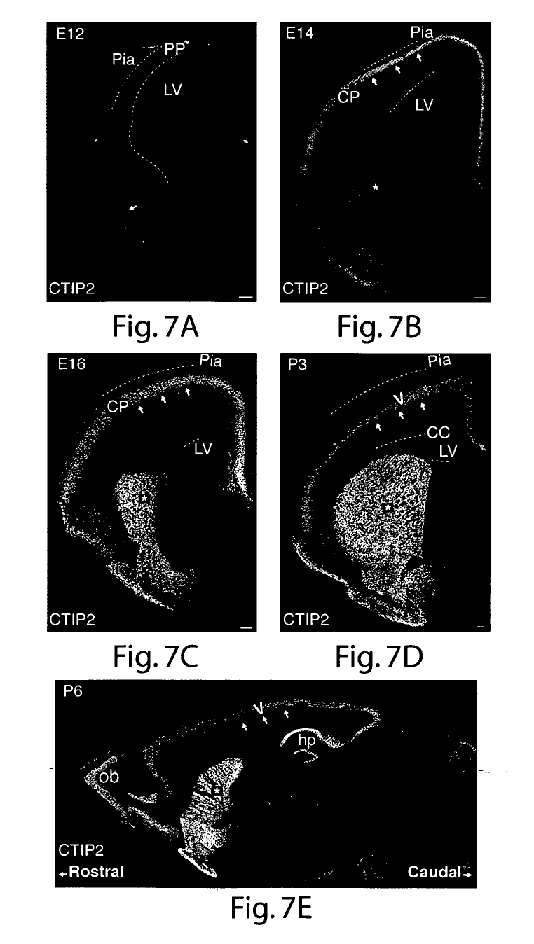

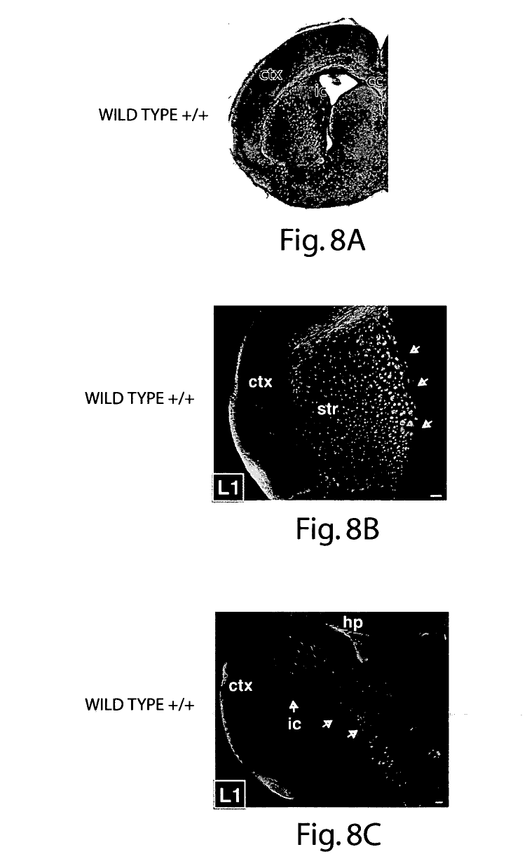

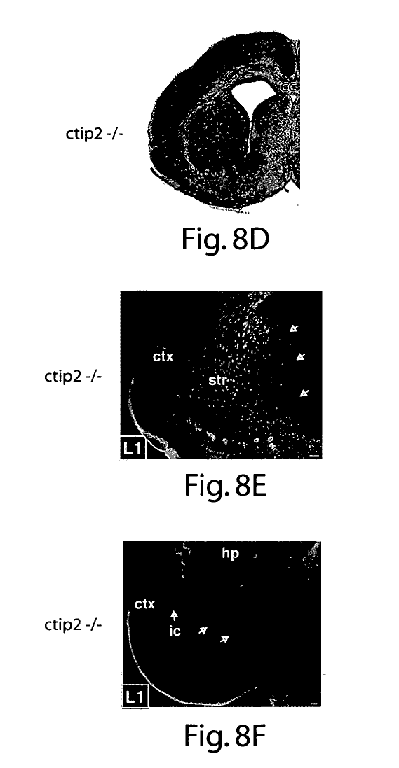

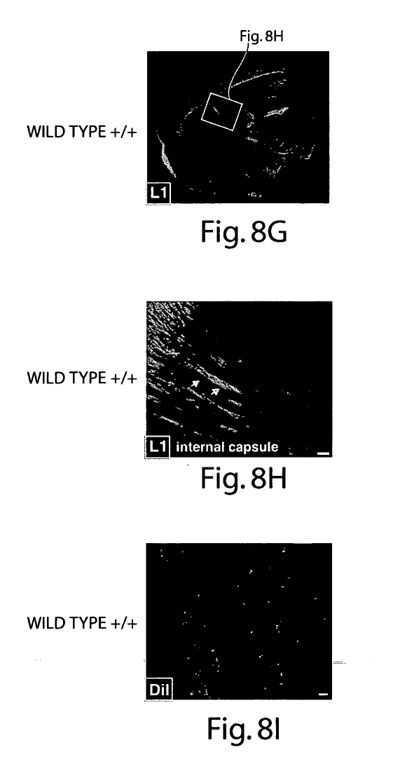

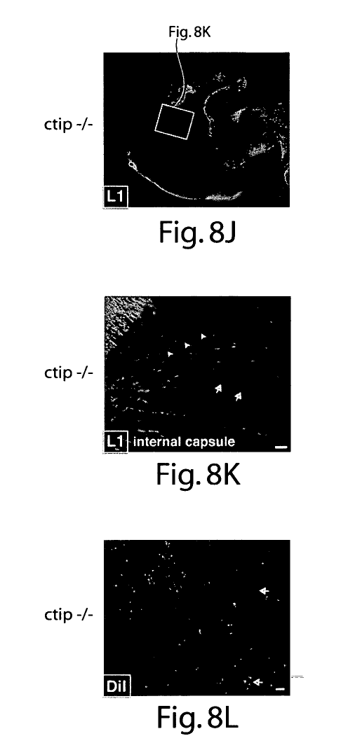

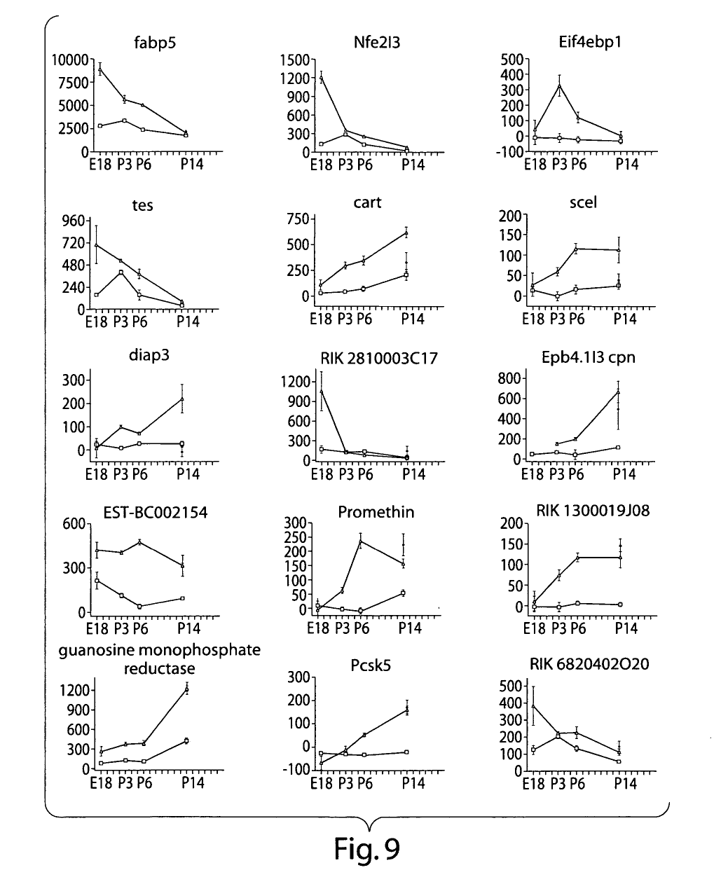

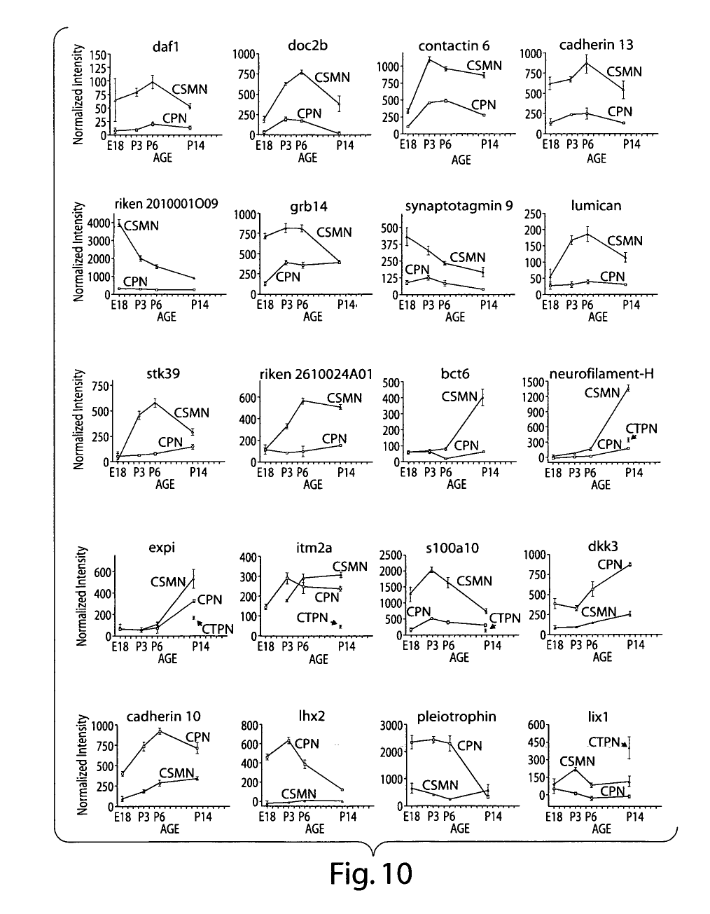

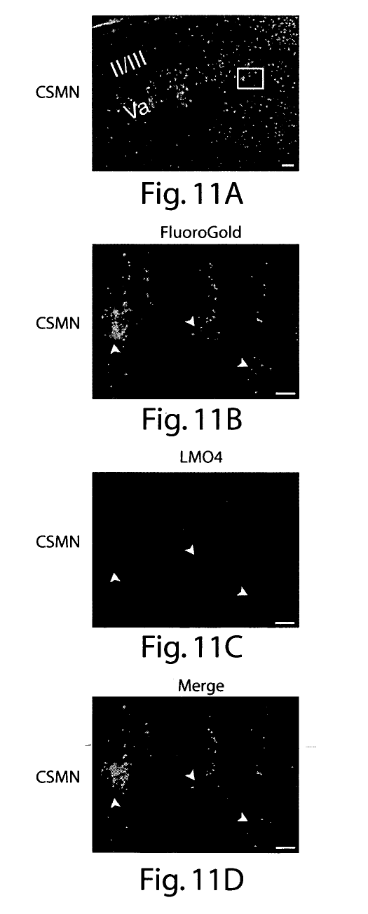

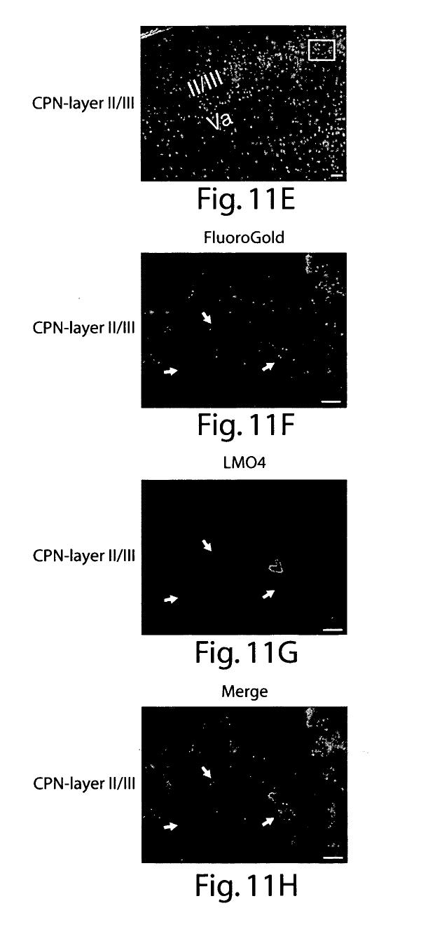

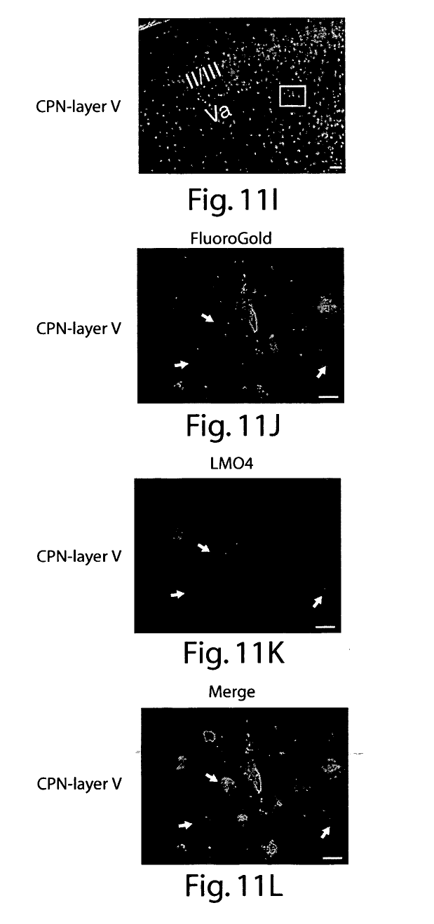

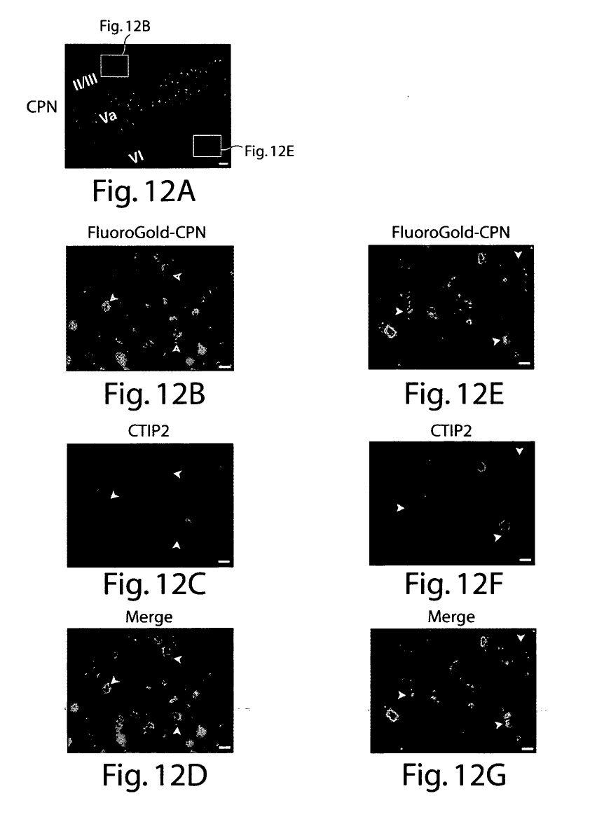

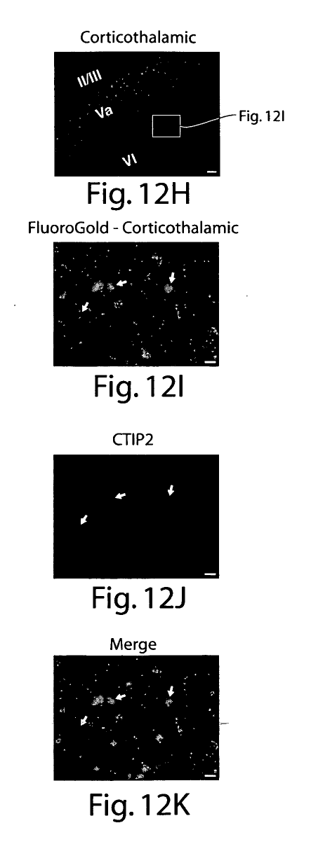

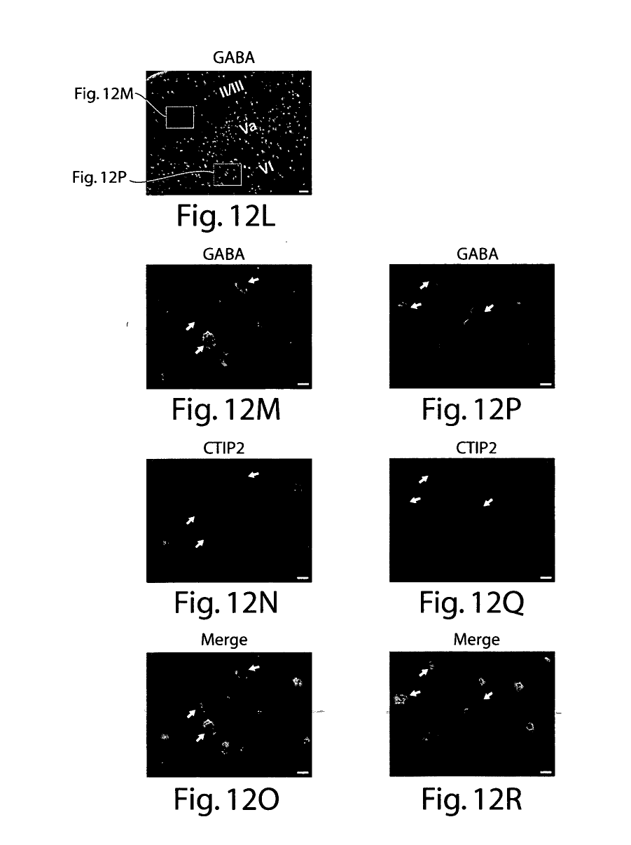

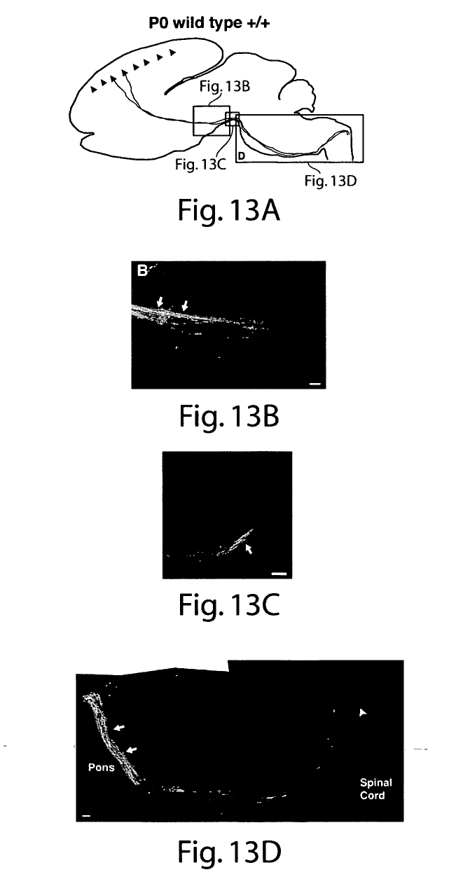

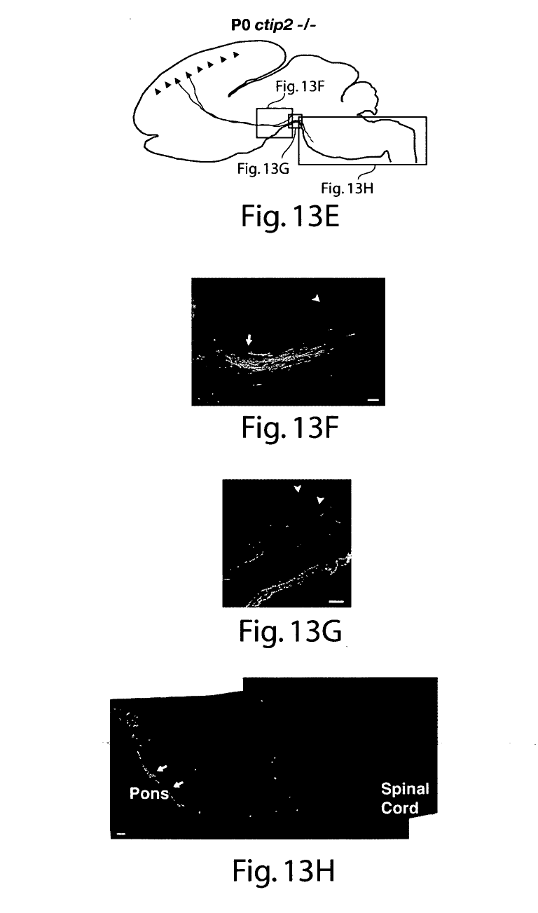

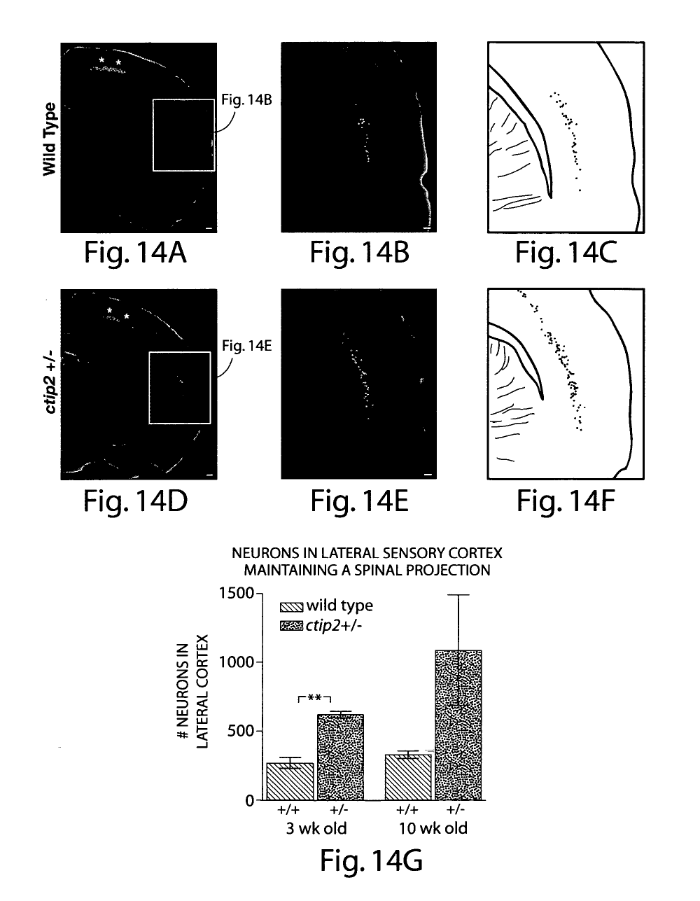

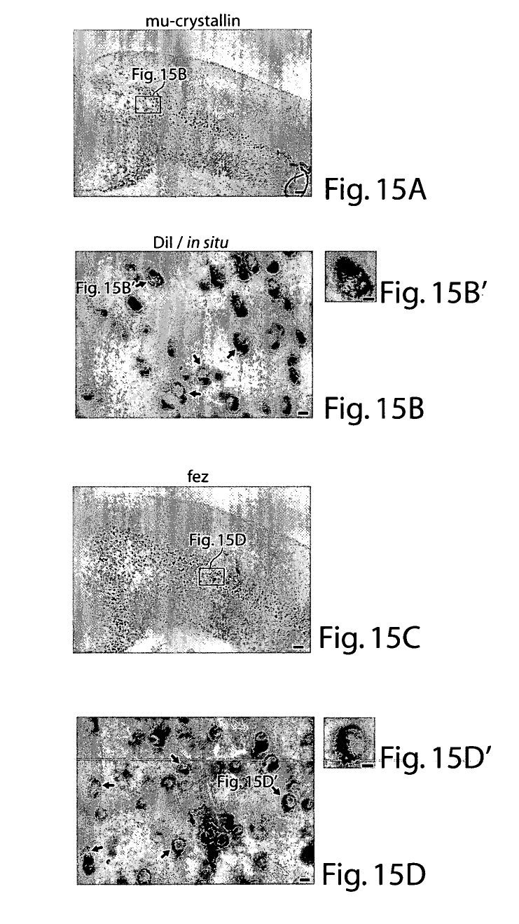

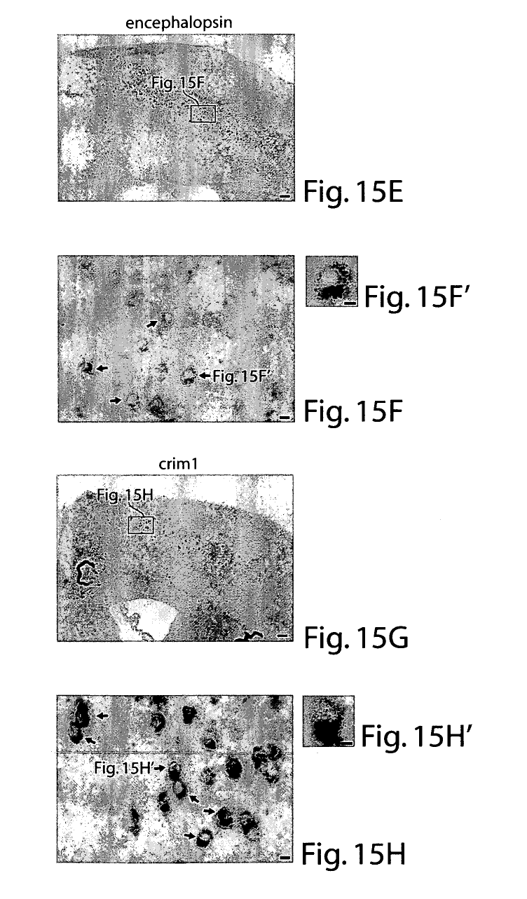

All neuronal subtypes were purified from C57BL/6 mice (Charles River Laboratories, MA). The day of vaginal plug was designated E0. CSMN were retrogradely labeled with green fluorescent microspheres (Lumafluor Corp., FL) injected into the pons-midbrain junction (E18), the pons (P3), or the cervical spinal cord at the C2-3 or C5 level for P6 and P14, respectively. For embryonic injections, E17 pregnant mice were deeply anesthetized with Avertin and each embryo was injected through the uterine wall into the pons, using a VisualSonics Vevo 660 ultrasound-guided microinjection system (VisualSonics; Toronto, Canada) to precisely control the position of the injection site (FIG. 1A-C). Two injections per embryo were performed, with a total of 60-80 nl of green fluorescent microspheres per injection site. The pregnant dam was deeply anesthetized one day after surgery, embryos were removed, and motor cortex was microdissected from both cerebral hemispheres using a fluorescence dissecting microscope to precisely visualize the labeled region (FIG. 1D). Similarly, for postnatal injections, P1, P4, and P1 mice were anesthetized by hypothermia (P1, P4) or Avertin (P11), and injected in the pons (P1) or the cervical spinal cord (P4 and P11). Four injections per pup were performed, with a total of 60-80 nl of green fluorescent microspheres per injection site. Pups were returned to the care of their mother and deeply anesthetized at P3, P6, and P14, respectively. Motor cortex was microdissected as described above.

Callosal neurons were labeled (at E18, P3, P6, and P14) as previously described, via injections of fluorescent microspheres into the corpus callosum (E17) or contralateral cortex (P1, P4, P12) (Catapano et al., 2001; Catapano, 2004). Corticotectal neurons were retrogradely labeled with green fluorescent microspheres injected into the superior colliculus of P11 pups. Pups were deeply anesthetized at P14, and visual cortex was microdissected using a fluorescence dissecting microscope, as described above. FluoroGold was injected into the cervical spinal cord or the contralateral sensorimotor cortex to label CSMN or callosal neurons, respectively, as previously described (Fricker-Gates et al., 2002). FluoroGold was injected into the thalamus of P4 mice under ultrasound guidance to label corticothalamic projection neurons. All animal studies were performed in accordance with institutional and federal guidelines.

CSM7, Corticotectal Neuron, and Callosal Neuron Dissociation and Purification.

Motor cortex (CSMN), visual cortex (corticotectal neurons), or sensorimotor cortex (callosal neurons), were dissected in cold dissociation medium [glucose (20 mM), Kynurenic acid (0.8 mM), APV (0.05 mM), penicillin-streptomycin (50 u/ml and 0.05 mg/ml, respectively), Na₂SO₄ (0.09M), K₂SO₄ (0.03M), MgCl₂ (0.014M)]. Retrogradely labeled cortex was enzymatically digested in dissociation medium containing L-cysteine HCl (0.016 μg/μL) and papain (10 u/ml; Worthington Biochemical Corp., NJ) at 37° C. for 30 min. Papain digestion was stopped with ovomucoid (10 mg/ml) and BSA (10 mg/ml) in dissociation medium at room temperature. Neurons were mechanically dissociated to obtain a single-cell suspension by gentle trituration in iced OptiMem containing glucose (20 mM), kynurenic acid (0.4 mM) and APV (0.025 mM). All chemicals were purchased from Sigma, and all media were purchased from Gibco-BRL, unless stated otherwise. Viability, assessed with trypan blue staining, was greater than 95% for E18, P3, and P6, and greater than 85% for P14 neurons.

Microsphere-labeled CSMN, callosal neurons, or corticotectal neurons were purified from the cortical cell suspension by fluorescence-activated cell sorting (FACS), using a BD FACS Vantage SE DiVa cell sorter directly into RNAlater® (Ambion). Cells were gated based on fluorescence, and forward and side scatter gates were set to select the population of large projection neurons shown in FIG. 2. P14 CSMN, callosal neurons, and corticotectal neurons were purified using a modified protocol that we developed for use with the more fragile late stage neurons. P14 tissue was enzymatically digested in papain (20 U/ml) for 45 minutes with constant stirring. DNase was added for the last 5 minutes of enzymatic digestion and to the media during trituration at 10 U/ml. Projection neurons were dissociated to a single cell suspension, as described above, and immediately preserved in RNAlater®. Cells were separated from debris by centrifuging at 5000×g for 45 minutes, and the pellet was resuspended in RNAlater® for FACS purification. FACS-sorted neurons from all ages and preparations were collected and stored in RNAlater® before RNA extraction (Barrett et al., 2002).

Affymetrix Microarrays

RNA was extracted using the StrataPrep Total RNA Micro Kit (Stratagene), and RNA quality was assessed using an Agilent Bioanalyzer system (Agilent Technologies). RNA was amplified according to the Affymetrix small sample protocol, using two consecutive rounds of linear in vitro transcription (IVT), to obtain 15-20 μg of amplified and labeled cRNA for each hybridization (Eberwine et al., 1992). To ensure reproducibility and biological significance, RNA samples were collected from two independent FACS purifications at each age (biological replicates). We used approximately 10,000 to 30,000 FACS-sorted neurons for each biological replicate, with the exception of P14 corticotectal neuron and E18 CSMN samples. For these samples, a minimum of 1000 neurons were used, because fewer neurons are labeled in these samples, leading to a smaller population of FACS-sorted neurons. Pearson's correlation coefficients of biological replicates were calculated using SAS software (SAS Institute). Because P14 neurons were fixed prior to FACS purification, while neurons at other ages were fixed after purification, we collected an additional biological replicate for P3 CSMN using the P14 method of fixation prior to sorting. The additional P3 replicate correlated highly with the other P3 samples (pair-wise correlation coefficients of 0.95 and 0.96), indicating that fixation of neurons prior to sorting did not affect microarray results.

Microarray Data Analysis

Data from all microarrays were normalized using two independent methods with Rosetta Resolver software (Rosetta Inpharmatics) by: 1) global scaling of each array intensity to the mean intensity value of all arrays; 2) the Rosetta Resolver error modeling method that more accurately normalizes transcripts expressed at low levels. Accurate normalization of the data reduces the rate of potential false differences introduced by using smaller numbers of cells in two of the samples. The robustness of the normalization methods is demonstrated by the fact that we did not detect differences in expression levels for known test genes in any of the samples, independent of starting cell number.

Statistical significance of gene expression differences between neuronal subtypes was determined by pair-wise comparisons at each age using SAM (Significance Analysis of Microarrays) (Tusher et al., 2001). Only genes scored as “present” in at least one microarray with a p-value<0.01 were included in the analysis. Fold change cut off was set at 3 for all comparisons, with the exception of the P14 CSMN versus P14 corticotectal neuron comparison, for which it was set at 2.0, because differences between these two subtypes at P14 are smaller in magnitude. We selected the 100 most statistically significant genes (highest d score) for each subtype at each age (total 884 unique genes). We further analyzed these genes for trends with likely biological relevance, 36 of which are described in FIG. 4 and FIG. 10. Microarray data from the two biological replicates were combined in Rosetta Resolver for trend plots. Analysis of data from both normalization methods yielded similar results.

Cluster Analysis

All cluster analysis was performed in Rosetta Resolver using an agglomerative hierarchical clustering method (Eisen et al., 1998). Unique genes scored as “present” in at least one experiment (p-value <10⁻⁵; total of 5904 genes) were included (FIG. 3).

Immunocytochemistry

Brains were fixed by transcardial perfusion with PBS-heparin (10 U/ml) followed by 4% paraformaldehyde, and post fixed overnight at 4° C. in 4% paraformaldehyde. 40 μm coronal or 60 μm saggital floating sections were blocked in 0.3% BSA (Sigma), 8% goat (Gibco) or donkey (Sigma) serum, and 0.3% Triton X-100 (Sigma) for 1 hour at room temperature, and incubated overnight at 4° C. in primary antibody. Primary antibodies and dilutions used were: rat anti-CTIP2, 1:500, a gift of M. Leid (Senawong et al., 2003); goat anti-LMO4 antibody, 1:200 (Santa Cruz Biotechnology); rabbit anti-GABA antibody, 1:1000 (Sigma); rabbit anti-TBRI, 1:500, a gift of R. Hevner; rabbit anti-ER81, 1:1000, a gift of T. Jessell (Arber et al., 2000); rabbit anti-L1 , 1:500, a gift of F. Rathjen (Brummendorf et al., 1998). Appropriate secondary antibodies were from the Alexa series (Molecular Probes). Laser confocal analysis was performed using a Biorad Radiance 2100 confocal microscope.

in situ Hybridization

Non-radioactive in situ hybridization was performed using reported methods (Berger and Hediger, 2001) using digoxigenin (DIG)-labeled cRNA probes on frozen coronal sections (10 μm) from P6 or P14 brains. Sense probes were used as negative controls in all experiments. We derived cDNA clones for encephalopsin, pcp4, mu-crystallin, csmn1, netrin-G1, and crim1 via RT-PCR, and cloned them into the Eco RI site of pCR II-TOPO (Invitrogen). To minimize the possibility that more than one transcript might be recognized by the same probe, we designed the PCR primers to amplify the same gene-specific regions that were used as targets in oligo design by Affymetrix, whenever possible. Each clone was sequenced on both strands, and compared to the GenBank database by BLAST to exclude the possibility of selecting cDNA regions shared by more than one gene. The fez clone was kindly provided by Dr. T. D. Sargent (NIH), and the clim1 clone was a gift of Dr. I. Bach (Hamburg University, Germany). The igfbp4 clone was obtained from the I.M.A.G.E consortium. All PCR primers and detailed information for each clone used for in situ hybridization are listed below:

| | | | SEQ | | |
|---|---|---|---|---|---|
| | | | ID | Amplicon | Plasmid |
| Clone Name | Source | PCR primers | NOs | Size | Vector |
| Igfbp4 | The | n/a | n/a | n/a | pCMV- |
| | I.M.A.G.E. | | | | SPORT 6 |
| | Consortium; | | | | |
| | clone # | | | | |
| | 5123738 | | | | |
| Crim1 | RT-PCR | TCTCAAGACTGTTGGTTGCTG; | 1; | 442 bp | pCR II |
| | | TGAACAACCAATGATAGCACAG | 2 | | TOPO |
| Fez | gift of Dr. T. | n/a | n/a | n/a | pT7T3D- |
| | D. Sargent, | | | | Pac |
| | NIH | | | | |
| Encephalopsin | RT-PCR | CTCACTGTGCTGGCCTATGA; | 3; | 523 bp | pCR II |
| | | GGGTTGTACACAGTGCTCGAT | 4 | | TOPO |
| Clim1 | gift of Dr. I. | n/a | n/a | n/a | pBluescript |
| | Bach, | | | | KS+ |
| | Hamburg | | | | |
| | University, | | | | |
| | Germany | | | | |
| Mu-Crystallin | RT-PCR | ACTGGCGAGAACTGGATGAC; | 5; | 436 bp | pCR II |
| | | GCCATCACCCCTTAACAGAA | 6 | | TOPO |
| Netrin G1 | RT-PCR | TGGCCGTTTCTTAAGACGAA; | 7; | 410 bp | pCR II |
| | | CTCAATTTCCCTGCTGCTGT | 8 | | TOPO |
| Csmn1 | RT-PCR | CCCTAGTTGGGACTGTGACC; | 9; | 367 bp | pCR II |
| | | AGCTCTGTTCACCCCACAAG | 10 | | TOPO |
| Pcp4 | RT-PCR | GGAGTCAGGCCAACATGAGT; | 11; | 432 bp | pCR II |
| | | TGCAGAGGAATTGTAATGGAGA | 12 | | TOPO |
| Cadherin 13 | RT-PCR | CTTCTAGTCGGGCAAGATGC; | 13; | 745 bp | pCR II |
| | | GGGTCTGTTGTCGTTCTGGT | 14 | | TOPO |
| S100a10 | RT-PCR | GCCCAGGTTTCGACAGACT; | 15; | 255 bp | pCR II |
| | | CCACTAGTGATAGAAAGCTCTGGA | 16 | | TOPO |
| Cadherin 22 | RT-PCR | CAAGCCCTATTCAGGAGCAG; | 17; | 872 bp | pCR II |
| | | CTTGGGGTCCACTGTGAAGT | 18 | | TOPO |
| Diap3 | RT-PCR | AGCAGTTCGCTGTTGTGATG; | 19; | 733 bp | pCR II |
| | | GCACGTTTCTCCTTTTCTGC | 20 | | TOPO |

ctip2−/− Mice

Homozygous ctip2−/− mice, described in Wakabayashi et al., 2003, have a neomycin resistance gene inserted into exon 1 of the ctip2 gene, inhibiting CTIP2 expression. Brains of P0 ctip2−/− mice were compared to those from wild type littermates using established methods (Lanier et al., 1999). Anterograde DiI tracing was performed as previously described (Godement et al., 1987; O'Leary and Terashima, 1988) by positioning DiI crystals in the neocortex. CSMN in sensorimotor and lateral sensory cortex were retrogradely labeled via FG injections in the cervical spinal cord. Mice were injected at P14-P15 and sacrificed at P21 or were injected and sacrificed as 10-week-old adults. Brains were sectioned coronally at 40 μm, and all CSMN (in sensorimotor and in lateral sensory cortex) were counted in both hemispheres on every sixth section, across the entire rostrocaudal extent of the cortex.

Results

Labeling and Purification of Corticospinal Motor Neurons

In order to identify genes that control cell-type specification and differentiation of the corticospinal motor neuron (CSMN) subtype, we compared gene expression profiles of CSMN, acutely isolated in vivo at four stages of development, to two other pure populations of cortical projection neurons—callosal projection neurons (CPN) and corticotectal projection neurons (CTPN) (Table 1).

TABLE 1
Summary of Microarray Experiments and Correlation Analysis.

| | | Biological | Correlation |
|---|---|---|---|
| Neuronal Subtype | Age | Replicates | Coefficient |
| CSMN | E18 | 2 | 0.94 |
| | P3 | 2 | 0.99 |
| | P6 | 2 | 0.99 |
| | P14 | 2 | 0.95 |
| CPN | E18 | 2 | 0.97 |
| | P3 | 2 | 0.99 |
| | P6 | 2 | 0.95 |
| | P14 | 2 | 0.99 |
| CTPN | P14 | 2 | 0.95 |

CSMN were retrogradely labeled by injecting green fluorescent microspheres into their axonal projection fields: the pons at early developmental stages (E18, P3) and the cervical spinal cord at later stages (P6, P14). Embryonic injections were performed using an ultrasound-guided microinjection system to accurately control the location of the injection site (FIG. 1A-C). This strategy specifically labels CSMN somas in motor cortex (FIG. 1D) based on their axonal projections, at four ages ranging from early post-mitotic (E18) to more differentiated (P3-P6), to more mature and synaptically integrated neurons (P14) (FIG. 1E-H). Similar techniques were used to label callosal neurons (FIG. 1I-L) and corticotectal neurons (FIG. 1M). Dissociated and labeled CSMN were then purified by FACS-sorting to typically >99% purity (FIG. 2A-F, F′).

CSMN were collected for RNA isolation immediately following FACS-purification. However, despite the fact that CSMN are very fragile neurons, under the optimized purification conditions used here, acutely FACS-sorted CSMN from E18, P3, and P6 cortex are alive, viable, and can be cultured in vitro (Ozdinler et al., unpublished observations), confirming their viability following FACS. Because neurons at P14 are even more fragile and difficult to FACS-purify without a substantial loss in cell viability, we developed a method for purifying these more mature neurons. P14 CSMN were fixed in RNAlater (see Experimental Procedures) immediately following dissociation, then purified by FACS using methods similar to those for earlier stages (FIG. 2E-F). After FACS-purification, these P14 CSMN still retain elements of their original in vivo morphology, including the proximal apical dendrite and occasionally the proximal axon (FIG. 2F′).

As alternate neuronal populations for comparison to CSMN, we used similar methods to purify interhemispheric callosal projection neurons, a subset of which share lamina V location with CSMN, thereby providing insight into genes that are cell-type specific rather than laminar specific; and corticotectal projection neurons, which share with CSMN both location in lamina V and overlapping early developmental extension of sub-cerebral projections. Corticotectal neurons might thus be useful for more detailed analysis of molecular pathways that are unique to CSMN among other, highly related, layer V sub-cerebral projection neurons. These data demonstrate that CSMN, as well as other subtypes of cortical neurons, can be acutely purified from the complex cellular environment of the neocortex at distinct and critical stages in vivo.

Cell-type Specific Expression Profile of Single Projection Neuron Subtypes

Gene expression analysis in the brain is generally complicated by the co-existence of many different cell types, resulting in high background noise and the inability to detect small differences in cell type-specific gene expression. The purification of single populations of cortical projection neurons allows us to overcome these difficulties and to compare pure, distinct neuronal populations for their expression profiles, without confounding contamination by other cell types (glia or other neurons). Here, we used pure populations of CSMN from E18, P3, P6, and P14 mice and compared them by microarray analysis to two other neuronal types, callosal neurons and corticotectal neurons. To control rigorously for biological sample variability, we used CSMN, callosal neurons, and corticotectal neurons from two independent neuronal FACS-isolations for each age analyzed (Table 1). We screened over 22,000 genes and ESTs using the Affymetrix M430A GeneChips (see Experimental Procedures). Correlation coefficients for biological replicates ranged from 0.94 to 0.99, indicating that our methods and amplification strategy are highly consistent and reproducible (Table 1).

Distinct Classes of Cortical Projection Neurons Share Clusters of Developmentally Regulated Genes

The development of CSMN is likely controlled by a combination of: 1) general molecular pathways common to all projection neurons; and 2) pathways that are CSMN specific. In order to identify genes in the first category, we used hierarchical clustering to examine changes in gene expression as cortical projection neurons differentiate and mature (FIG. 3A). We found that there exist distinct groups of genes that are expressed with similar profiles in different projection neuron subtypes (FIG. 3B-D). Because these genes are expressed in CSMN and other subtypes of cortical projection neurons with a similar profile of developmental regulation, they are likely to play critical general roles in the development of cortical projection neurons.

At the earliest developmental stage (E18), these genes include molecules highly expressed in both CSMN and callosal neurons (FIG. 3B), and, thus, these may be part of a general genetic program of early projection neuron specification and differentiation. Additionally, we found a large cluster of genes that are up-regulated at P14 in CSMN, callosal neurons, and corticotectal neurons and, thus, may have a general role in establishing connectivity, synaptic integration, and mature neuronal function of projection neurons (FIG. 3D).

Another finding from this initial cluster analysis is that, at P14, CSMN are molecularly very similar to the corticotectal neuron subtype, while both are distinct from the callosal neuron subtype (data not shown). This likely reflects the fact that both CSMN and corticotectal neurons may have similar requirements for survival (both are very large and distally connected neurons) and connectivity (both have very long-distance sub-cerebral axonal connections), which in turn may result in common molecular controls over these events. These data are in agreement with and support earlier reports by O'Leary and colleagues showing that, while callosal neurons never extend axonal projections to sub-cerebral targets, both CSMN and corticotectal neurons initially make projections toward the same sub-cerebral targets, and only later diverge and connect to the spinal cord and the superior colliculus, respectively (O'Leary and Koester, 1993). These primary data suggested that CSMN and corticotectal neurons arise from closely related or the same cells and share many developmental events that are different from callosal neurons. We now provide direct molecular evidence that supports and extends this hypothesis.

Identification of Corticospinal Motor Neuron-Specific Genes

To identify CSMN-specific genes, we determined the significance of the differences in gene expression among neuronal subtypes by pair-wise comparisons at each age, using the SAM (Significance Analysis of Microarray) method, which accounts for the probability of false positives and ranks significance based on both the level of differential expression and the standard deviation across different measurements (d score) (see Experimental Procedures). We selected the 100 most significant genes (highest d scores) from each pair-wise comparison of all three neuronal populations performed at each age (total of 884 unique genes), and further analyzed the trend of expression of each individual gene to define a smaller set of molecules of potentially high biological relevance.

We identified genes that are specifically expressed in CSMN, as well as genes that are specific to callosal neurons and corticotectal neurons that can, importantly, serve as negative molecular markers of CSMN. Of the many genes identified, a selection of the most biologically interesting and statistically significant genes are described in FIG. 4. These genes can be classified into one of six groups based on expression profiles suggestive of a specific role in distinct aspects of CSMN development. The six groups are: 1) genes that are expressed at higher levels in CSMN at all stages of development (FIG. 4A) and may be important for the establishment and maintenance of CSMN identity; 2) genes that are highly expressed in CSMN early in development (FIG. 4B) and might be important for early CSMN specification; 3) and 4) genes that exhibit increasing levels of expression as CSMN develop and may control intermediate and later aspects of CSMN differentiation, such as process outgrowth and synapse formation (FIGS. 4C,D); 5) genes that are expressed at higher levels in CSMN compared to the highly related population of corticotectal neurons (FIG. 4E) and are representative of the small class of genes that differentiate CSMN from other sub-cerebral projection neurons of layer V; 6) genes that are negative markers of CSMN, but are expressed in callosal or corticotectal neurons (FIG. 4F). Expression trends of all these genes are shown in FIG. 10.

The CSMN genes we identified include, among many other statistically significant genes that were omitted from FIG. 4 for clarity and simplicity: transcription activators and repressors (e.g. ctip2, bc16, sox5); zinc-finger domain-containing proteins (e.g. fez); cell surface proteins and receptors (e.g. encephalopsin, itm2a, daf1); calcium signaling molecules (e.g. pcp4, s100a10); genes involved in neuronal specification (e.g. crim1), cell adhesion (e.g. cdh22, cdh13, cntn6), and axon guidance (e.g. neto1, netrin G1); as well as genes involved in critical pathways like the thyroid hormone and IGF signaling cascades (e.g. mu-crystallin, igfbp4).

CSMN-specific genes are further summarized in Table 2. Additional CSMN-specific genes are listed in Table 3, and expression trends for these genes are shown in FIG. 9. CSMN-specific and CSMN-excluded genes are listed according to function in Table 4. CSMN-excluded genes are listed in Table 5.

TABLE 2
Summary of CSMN-specific Genes

| | | Genbank |
|---|---|---|
| | | Accession |
| Name | Full name | Number |
| Daf1 | decay accelerating factor 1 | NM_010016 |
| Fez | forebrain embryonic zinc | NM_080433 |
| | finger | |
| Doc2b | double C2, beta | NM_007873 |
| Cntn6 | contactin 6 | NM_017383 |
| Cdh13 | cadherin 13 | NM_019707 |
| B430320C24Rik | RIKEN cDNA B430320C24 | AK046712 |
| | gene | |
| Grb14 | growth factor receptor | NM_016719 |
| | bound protein 14 | |
| Ldb2/clim1 | LIM domain binding 2 | NM_010698 |
| Syt9 | synaptotagmin 9 | NM_021889 |
| 2010001O09Rik | RIKEN cDNA 2010001O09 | NM_025909 |
| | gene | |
| Sox5 | SRY-box containing gene 5 | NM_011444 |
| Ctip2/Bcl11b | COUP-TF interacting | AF186019 |
| | protein 2 | |
| Crym | crystallin, mu | NM_016669 |
| Stk39 | serine/threonine kinase 39 | NM_016866 |
| Lum | lumican | NM_008524 |
| RIKEN 2610024A01 | | XM_129450 |
| Crim1 | cysteine-rich motor neuron 1 | XM_128751 |
| Neto1 | neuropilin (NRP) and | NM_144946 |
| | tolloid (TLL)-like 1 | |
| Ecpn | Encephalopsin | AF140241 |
| Bcl6 | B-cell leukemia/lymphoma 6 | BC052315 |
| Cdh22 | cadherin 22 | AB019618 |
| Csmn1 | RIKEN cDNA 1110032O19 | AI430822 |
| | gene | |
| Ntng1 | netrin G1 | NM_030699 |
| Pcp4 | Purkinje cell protein 4 | NM_008791 |
| Expi | extracellular proteinase | NM_007969 |
| | inhibitor | |
| S100a10 | S100 calcium binding pro- | NM_009112 |
| | tein A10/Calpactin I light | |
| | chain | |
| Ramp3 | receptor activity modifying | NM_019511 |
| | protein 3 | |
| Itm2a | integral membrane pro- | NM_008409 |
| | tein 2A | |
| NF-H | neurofilament, heavy poly- | M35131 |
| | peptide | |
| Igfbp4 | insulin-like growth factor | NM_010517 |
| | binding protein 4 | |

TABLE 3
Additional CSMN specific genes

| | | GenBank |
|---|---|---|
| | | accession |
| Name | Full name | number |
| Fabp5 | fatty acid binding protein 5, | BC002008 |
| | epidermal | |
| Nfe2l3 | nuclear factor, erythroid | NM_010903 |
| | derived 2, like 3 | |
| Eif4ebp1 | eukaryotic translation initia- | NM_007918 |
| | tion factor 4E binding protein 1 | |
| tes | testis derived transcript | BC010465 |
| Cart | cocaine and amphetamine regu- | NM_013732 |
| | lated transcript | |
| scel | sciellin | NM_022886 |
| Diap3 | diaphanous homolog 3 | NM_019670 |
| RIKEN 2810003C17 | | BC024599 |
| Epb4.1l3 | erythrocyte protein band | AF177146 |
| | 4.1-like 3 | |
| EST | | BC002154 |
| Promethin | | NM_145586 |
| RIKEN 1300019J08 | | AK005066 |
| Riken 2210012L08 | RIKEN cDNA 2210012L08 gene | AK008716 |
| | guanosine monophosphate | NM_025508 |
| | reductase | |
| Pcsk5 | proprotein convertase | BC013068 |
| | subtilisin/kexin type 5 | |
| RIKEN 6820402O20 | | BB308157 |

TABLE 4
Genes Associated with Function

| | | | GenBank |
|---|---|---|---|
| | Shortened | | accession |
| Function | name | Full gene name | number |
| CSMN axon guidance/process | Cntn6 | contactin 6 | NM_017383 |
| outgrowth | | | |
| CSMN axon guidance/process | Cdh13 | cadherin 13 | NM_019707 |
| outgrowth | | | |
| CSMN axon guidance/process | Ldb2 | LIM domain binding 2 | NM_010698 |
| outgrowth | | | |
| CSMN axon guidance/process | 2010001O09Rik | RIKEN cDNA 2010001O09 | NM_025909 |
| outgrowth | | gene | |
| CSMN axon guidance/process | Ctip2/Bcl11b | COUP-TF interacting protein 2 | AF186019 |
| outgrowth | | | |
| CSMN axon guidance/process | Neto1 | neuropilin (NRP) and tolloid | NM_144946 |
| outgrowth | | (TLL)-like 1 | |
| CSMN axon guidance/process | Cdh22 | cadherin 22 | AB019618 |
| outgrowth | | | |
| CSMN axon guidance/process | Ntng1 | netrin G1 | NM_030699 |
| outgrowth | | | |
| CSMN end stage differentiation | Ecpn | Encephalopsin | AF140241 |
| CSMN end stage differentiation | Bcl6 | B-cell leukemia/lymphoma 6 | BC052315 |
| CSMN end stage differentiation | CSMN1 | RIKEN cDNA 1110032O19 | AI430822 |
| | | gene | |
| CSMN end stage differentiation | Expi | extracellular proteinase | NM_007969 |
| | | inhibitor | |
| CSMN end stage differentiation | S100a10 | S100 calcium binding protein | NM_009112 |
| | | A10/Calpactin I light chain | |
| CSMN end stage differentiation | Itm2a | integral membrane protein 2A | NM_008409 |
| CSMN end stage differentiation | NF-H | neurofilament, heavy | M35131 |
| | | polypeptide | |
| CSMN end stage differentiation | Cart | cocaine and amphetamine | NM_013732 |
| | | regulated transcript | |
| CSMN end stage differentiation | Epb4.1l3 | erythrocyte protein band 4.1- | AF177146 |
| | | like 3 | |
| CSMN end stage differentiation | Syt9 | synaptotagmin 9 | NM_021889 |
| CSMN end stage differentiation | scel | sciellin | NM_022886 |
| CSMN end stage differentiation | Diap3 | diaphanous homolog 3 | NM_019670 |
| CSMN end stage differentiation | Promethin | | NM_145586 |
| CSMN fate specification | Fez | forebrain embryonic zinc | NM_080433 |
| | | finger | |
| CSMN fate specification | B430320C24Rik | RIKEN cDNA B430320C24 | AK046712 |
| | | gene | |
| CSMN fate specification | Sox5 | SRY-box containing gene 5 | NM_011444 |
| CSMN fate specification | Fabp5 | fatty acid binding protein 5, | BC002008 |
| | | epidermal | |
| CSMN fate specification | Nfe2l3 | nuclear factor, erythroid | NM_010903 |
| | | derived 2, like 3 | |
| CSMN fate specification | Crim1 | cysteine-rich motor neuron 1 | XM_128751 |
| CSMN fate specification | tes | testis derived transcript | BC010465 |
| CSMN survival | Grb14 | growth factor receptor bound | NM_016719 |
| | | protein 14 | |
| CSMN survival | Crym | crystallin, mu | NM_016669 |
| CSMN survival | Pcp4 | Purkinje cell protein 4 | NM_008791 |
| CSMN survival | Ramp3 | receptor activity modifying | NM_019511 |
| | | protein 3 | |
| CSMN survival | Igfbp4 | insulin-like growth factor | NM_010517 |
| | | binding protein 4 | |
| | Daf1 | decay accelerating factor 1 | NM_010016 |
| | Doc2b | double C2, beta | NM_007873 |
| | Stk39 | serine/threonine kinase 39 | NM_016866 |
| | Lum | lumican | NM_008524 |
| | RIKEN | | XM_129450 |
| | 2610024A01 | | |
| | Eif4ebp1 | eukaryotic translation initiation | NM_007918 |
| | | factor 4E binding protein 1 | |
| | RIKEN | | BC024599 |
| | 2810003C17 | | |
| | EST | | BC002154 |
| | RIKEN | | AK005066 |
| | 1300019J08 | | |
| | Riken | RIKEN cDNA 2210012L08 | AK008716 |
| | 2210012L08 | gene | |
| | | guanosine monophosphate | NM_025508 |
| | | reductase | |
| | Pcsk5 | proprotein convertase | BC013068 |
| | | subtilisin/kexin type 5 | |
| | RIKEN | | BB308157 |
| | 6820402O20 | | |
| CPN axon guidance | Lmo4 | LIM domain only 4 | NM_010723 |
| CPN axon guidance | Cdh10 | cadherin 10 | AF183946 |
| CPN end stage differentiation | Dkk3 | dickkopf homolog 3 | NM_015814 |
| CPN fate spec | Lhx2 | LIM homeobox protein 2 | NM_010710 |
| CPN survival/differentiation | Ptn | pleiotrophin | BC002064 |
| CTPN | Lix1 | limb expression 1 homolog | NM_025681 |
| | | (chicken) | |

TABLE 5
CSMN-excluded genes

| | | |
|---|---|---|
| Ptn | pleiotrophin | BC002064 |
| Lhx2 | LIM homeobox protein 2 | NM_010710 |
| Lmo4 | LIM domain only 4 | NM_010723 |
| Lix1 | limb expression 1 homolog (chicken) | NM_025681 |
| Cdh10 | cadherin 10 | AF183946 |
| Dkk3 | dickkopf homolog 3 | NM_015814 |

To confirm CSMN-specific gene expression and validate the microarray data, we performed in situ hybridization or immunocytochemistry for 14 selected genes (FIG. 4, bolded) chosen for their particularly interesting patterns of expression and potential function based on protein domains or literature from other systems. We chose diap3 (Olson, 2003); igfbp4 (Stenvers et al., 1994); crim1 (Kolle et al., 2000); ctip2 (Avram et al., 2000); encephalopsin (Blackshaw and Snyder, 1999); clim1 (also known as ldb2) (Bulchand et al., 2003); fez (Matsuo-Takasaki et al., 2000); pcp4 (Sangameswaran et al., 1989); s100a10 (Saris et al., 1987); mu-crystallin (Segovia et al., 1997); netrin-G1 (Yin et al., 2002); cadherin 13 (Huang et al., 2003); cadherin 22 (Sugimoto et al., 1996); and one novel EST that we name csmn1. These are all largely undescribed molecules in cortex that have microarray expression profiles strongly indicating subtype-specific expression in CSMN and/or other sub-cerebral projection neurons (FIG. 5B′,D′-P′).

We find that all these genes have high levels of expression in layer V of cortex, where they are strongly expressed in morphologically identified CSMN (FIG. 5A-P), confirming and extending the microarray results. By in situ hybridization, these genes show different degrees of restriction to CSMN. Three genes—diap3, igfbp4, and crim1—demonstrate particularly interesting and very restricted patterns of expression that distinguish CSMN from other sub-cerebral projection neurons. Diap3 is expressed only in sensorimotor layer V where CSMN are located, while it is not expressed in more lateral (FIG. 5A) or caudal (FIG. 5B) areas of layer V where other sub-cerebral projection neurons (e.g. corticotectal neurons) are located. Igfbp4 exhibits a similar degree of restriction to CSMN in sensorimotor layer V (FIGS. 5C,D), although it is also expressed in other populations in layers II/III and VI. Crim1 is restricted to layer V, with high level expression in rostral sensorimotor cortex (FIG. 5E). These three genes appear to be area-specific markers that identify the location of CSMN in layer V along medio-lateral and rostro-caudal axes.

A larger group of genes—ctip2, encephalopsin, clim1, fez, pcp4, and s100a10—appear to be expressed in CSMN and the broader class of closely related sub-cerebral projection neurons in layer V (FIG. 5F-K). In contrast, mu-crystallin and netrin-G1 appear to be expressed only in some CSMN and sub-cerebral neurons of layer V (FIGS. 5L,M) and may delineate distinct functional classes. Consistent with the microarray data, other genes—csmn1, cadherin 13 and cadherin 22—show less restricted patterns of expression, but are expressed at much higher levels in sub-cerebral neurons (FIG. 5N-P). Together, these data support the hypothesis that a small number of CSMN restricted genes, together with a larger group of genes that are also expressed in other sub-cerebral neurons, define the molecular phenotype of CSMN.

To further confirm the cellular identity and projection neuron type of labeled cells identified by in situ hybridization, we combined in situ hybridization with retrograde labeling in the same tissue. We retrogradely labeled CSMN with DiI, photoconverted the DiI to a visible cytoplasmic precipitate, and performed non-radioactive in situ hybridization. Combining these methods allows the co-localization of the DiI photoconverted precipitate and the in situ hybridization signal, enabling us to identify CSMN expressing individual genes. We investigated four genes from FIG. 5 and confirmed that CSMN express mu-crystallin (FIGS. 15A,B,B′),fez (FIGS. 15C,D,D′), encephalopsin (FIGS. 15E,F,F′), and crim1 (FIGS. 15G,H,H′).

As cell fate specification and maturation of CSMN will almost certainly depend on both positive and negative molecular determinants of neuronal subtype, we also included and further characterized by immunocytochemistry one additional gene, lmo4 (FIG. 11A), a LIM-domain-containing protein that is known to be expressed in layer II/III and V (Bulchand et al., 2003), although its precise cell-specific expression in different neuron types within those cortical layers was not previously known. Lmo4, together with other genes expressed in callosal or corticotectal neurons but not in CSMN (FIG. 4F and data not shown), can serve as negative genetic markers of CSMN.

These data provide strong evidence that we identified novel or previously uncharacterized genes that are specific to CSMN. These genes likely play important functional roles in the specification and/or development of this clinically important neuronal subtype. In addition, these same molecules can potentially be used as positive markers of CSMN. Of interest, while they all label CSMN, these molecules have different patterns of expression, and it is likely their combinatorial interaction that defines CSMN. Together with negative markers, they will allow the progressive definition of the molecular phenotype of CSMN in vivo.

Lmo4 is not Expressed in CSMN and is Restricted to Callosal Neurons in Layer V

Our microarray data indicate that lmo4 is specifically expressed in callosal neurons, but not in CSMN (FIG. 4F). These data, together with immunocytochemistry data showing expression of LMO4 in layer V (FIGS. 11A and C), where both CSMN and callosal neurons reside, strongly suggest that LMO4 is a neuronal subtype-specific, rather than a layer-specific, molecule and could contribute, together with other subtype-restricted genes, to specify distinct neuronal identity in vivo.

To definitively test the hypothesis that LMO4 is not expressed in CSMN, but, rather, is restricted to callosal neurons in layer V, we retrogradely labeled CSMN and callosal neurons via FluoroGold injections into their axonal fields, and performed co-localization analysis with LMO4 immunocytochemistry. We found that none of the CSMN in layer V (FluoroGold-labeled from spinal cord) express LMO4 (FIG. 11A-D). In contrast, all callosal neurons in both layer II/III and layer V (FluoroGold-labeled from contralateral cortex) express LMO4 at high levels (FIG. 11E-L). These data demonstrate that LMO4 is a neuronal subtype-restricted gene and, most important to the present study, is a negative marker of the CSMN population that can be used to distinguish CSMN from other neuronal types within layer V.

CTIP2 (COUP-TF1 interacting protein 2) is Expressed in CSMN but not in Callosal Neurons in Layer V

To begin to understand the functional roles of selected CSMN-specific molecules, we characterized more precisely the CSMN expression of ctip2, a gene of yet unknown function in the brain that shows a very high level of expression in layer V in both CSMN and corticotectal neurons (FIGS. 5C,C′ and 6A). Very recent studies have shown that CTIP2 has critical roles in the immune system, controlling T-cell subtype specification and survival in the developing thymus (Wakabayashi et al., 2003). These data suggested to us that it might have similar, yet undiscovered, roles in the specification, maintenance, and/or connectivity of distinct neuronal populations in the nervous system, specifically of CSMN and other sub-cerebral projection neurons. The idea that some of the same molecules that control the formation and/or maintenance of distinct cell types during the development of the immune system also control lineage determination/survival in the nervous system is a recent and very intriguing one, increasingly supported by several lines of evidence (Huh et al., 2000).

To test the hypothesis that CTIP2 may control some aspects of CSMN and sub-cerebral projection neuron development, we first defined its cell type-specific pattern of expression in the cortex, and confirmed its expression in CSMN and corticotectal neurons. We found that CTIP2 is expressed at high levels in layer V of cortex in a pattern that extends across the entire rostro-caudal (FIG. 6A) and medial-lateral (data not shown) aspects of the cortex. This is consistent with our microarray data, showing high levels of expression in both CSMN and corticotectal neurons. Since CTIP2 expression extends outside of the boundaries of motor cortex, but is specific to layer V, we hypothesized that CTIP2 is expressed at high levels in all sub-cerebral projection neurons, but not in cortico-cortical projection neurons (e.g. callosal neurons) or in other locally integrated neurons of layer V.

To test this hypothesis, we performed a series of experiments in which we selectively retrogradely labeled individual populations of projection neurons. First, we injected FluoroGold into the pyramidal tract at the pons-midbrain junction, to label sub-cerebral projection neurons (FIG. 6B). We found that all sub-cerebral projection neurons located in layer V express CTIP2 at high levels (FIGS. 6C and 6D-G). Next, we labeled CSMN specifically via FluoroGold injections into the cervical spinal cord and confirmed that all CSMN express CTIP2 at high levels (FIG. 6H-K). Conversely, FluoroGold labeling of callosal neurons via injection in contralateral cortex revealed that CTIP2 is not expressed by the relatively small number of callosal neurons in layer Va, where CTIP2 expression is highest (FIG. 6L-O), nor by callosal neurons in layer II/III (FIG. 12A-D) or layer VI (FIG. 12E-G). Together, these data demonstrate that CTIP2 is a neuronal subtype-specific, not simply a layer-specific, marker of CSMN and other evolutionarily-related populations of neurons with sub-cerebral projections.

Of additional interest, together with the predominant population of sub-cerebral projection neurons in layer Va that express CTIP2 at very high levels, we also observed a set of neurons in superficial and deep cortical layers that express CTIP2 at much lower levels. Many of the low level CTIP2 expressing neurons in cortical layer VI possess nuclei morphologically stereotypical for subcortical projection neurons. We hypothesized that these neurons are corticothalamic projection neurons.

To test this hypothesis, we labeled corticothalamic projection neurons by thalamic injections of FluoroGold at P4 and investigated whether these layer VI neurons are corticothalamic projection neurons expressing CTIP2 at low levels. We found that all corticothalamic projection neurons express CTIP2, although at much lower levels than layer V CSMN and other sub-cerebral projection neurons (FIG. 12H-K).

In addition, based on distribution across the cortex and nuclear morphology, we hypothesized that another population of neurons in layers II/III and VI expressing CTIP2 at low levels might be GABAergic interneurons. This was supported by our observation of high levels of CTIP2 expression ventro-lateral to the developing ganglionic eminences from which GABAergic interneurons originate (FIG. 7A). GABA immunocytochemistry confirmed that essentially all of the neurons in layer II/III with weak CTIP2 expression are indeed GABAergic (FIG. 12L-O). A subset of deep layer VI CTIP2 positive cells are also GABAergic (FIG. 12P-R). It is possible that these populations of GABAergic neurons derived from the ganglionic eminence might form elements of local circuitry with the sub-cerebral projection neurons, including CSMN, that express CTIP2 at high levels, or be developmentally linked with these neurons during laminar specification.

Together, these data demonstrate that CTIP2 is expressed in a tightly regulated pattern in specific populations of sub-cerebral projection neurons. High-level expressing neurons of the cortex are all long distance projection neurons of layer V projecting sub-cerebrally, and include CSMN. All of these CTIP2 positive neurons extend their axons through the internal capsule toward sub-cerebral targets. Interestingly, layer VI corticothalamic neurons, which are weakly positive for CTIP2, also extend their axons through the internal capsule before reaching the thalamus. This suggests that CTIP2 may be involved in controlling aspects of CSMN connectivity, including axonal outgrowth and guidance at a time when CSMN project their axons to the same sub-cerebral targets (internal capsule, pons, medulla) as other classes of CTIP2-positive layer V projection neurons, before CSMN axons uniquely descend through the spinal cord. This hypothesis is supported by the fact that the expression of CTIP2 sharply decreases at later stages of development (FIGS. 5C′ and 7; P14 immunocytochemistry data not shown).

CTIP2 is Expressed in the Developing Cortical Plate and in CSMN in layer V, but is Excluded from Progenitors in the Ventricular and Subventricular Zone

To understand better the functional role of CTIP2 in CSMN development, we investigated its temporal course of expression through embryonic and postnatal cortical development. As shown in FIG. 7A, at E12, when early cortical progenitors are dividing, CTIP2 is expressed in only a small cluster of cells ventro-lateral to the developing ganglionic eminences. In contrast, no cells expressing CTIP2 are visible in either the ventricular zone or the subventricular zone, where cortical neural precursors and committed progenitors are located that are thought to give rise to cortical projection neurons. This suggests that CTIP2 is likely not involved in the early specification of cortical progenitors. At E14, during peak production of CSMN, and at E16, when the majority of CSMN have reached the cortical plate, CTIP2 is highly expressed by cells in the cortical plate, but not by cells in the ventricular zone or the subventricular zone, suggesting that CTIP2 begins to be expressed in post-mitotic CSMN and other sub-cerebral projection neurons once they reach the cortical plate (FIGS. 7B,C). These data suggest that CTIP2 might control final CSMN positioning in the correct cortical layer, or, alternatively, CSMN post-mitotic differentiation, including process outgrowth and pathfinding and/or survival. The second hypothesis appears more likely, in light of the fact that: 1) CTIP2 is not expressed in other neurons that also take position in developing layer V (i.e. callosal neurons); rather, CTIP2 exhibits restricted expression to CSMN and other related neurons with similar long-distance sub-cerebral connections (FIG. 6); 2) high levels of CTIP2 expression are observed in post-mitotic immature neurons that have just started to extend an axon (E14-E18); and 3) mice with a targeted deletion of COUP-TF1 (a major interacting protein of CTIP2) display axonal pathfinding defects (Zhou et al., 1999).

An alternative, but not exclusive, possibility is that CTIP2 controls CSMN survival at a time when they have reached their final location in layer V and before they have formed stable connections to final distal targets in the spinal cord, which would then provide target-derived trophic support. This second hypothesis is supported by the observations that: 1) CTIP2 expression is maintained at high levels during progressive morphological maturation (P3 and P6; FIGS. 7D,E), prior to target innervation and trophic support, after which expression decreases significantly by P14 (FIG. 5C′ and data not shown); and 2) lack of the ctip2 gene is associated with massive cell death in the developing thymus in vivo (Wakabayashi et al., 2003).

Ctip2−/− Mice Have Abnormal Cortical Fiber Tracts

In order to test these alternative hypotheses and define the function of CTIP2 in vivo, we investigated homozygous ctip2−/− mice, in which the neomycin (neo) resistance gene was inserted into exon 1 of the ctip2 gene, inhibiting CTIP2 expression (Wakabayashi et al., 2003). These mice are born alive, but die soon after birth (P0), with defects in the survival of the specific lineage of immune yδT-cells and acute apoptosis in the developing thymus (Wakabayashi et al., 2003).

To investigate whether the neocortex of ctip2−/− mice has any abnormalities, we compared the cortical architecture of P0 ctip2−/− mice to wild type littermate controls. We labeled cortical layers using markers of distinct cortical laminae: LMO4 (layer II/III and V); ER81 (layer V); and TBR1 (layer VI). At the fairly high level of cellular resolution of these markers, the cortex of ctip2−/− mice appears normal, suggesting that the lack of ctip2 does not result in widespread death of distinct neuronal populations, nor in neuronal lamination defects (data not shown). While there exists the possibility that more subtle cytoarchitectural abnormalities are present and/or that neuronal cell death may occur at later developmental stages, the early neonatal lethal phenotype of ctip2−/− mice makes it currently difficult to test these theoretical possibilities.

Very interestingly, however, despite the fact that ctip2−/− mice die at P0, well before CSMN axons have connected to their final targets in the spinal cord, ctip2−/− mice display striking abnormalities of axonal fiber tracts exiting the neocortex and forming the internal capsule. Specifically, ctip2−/− mice have substantial disorganization of the anatomically distinct pattern of cortical axon fascicles that normally perforate the striatum to form the internal capsule (FIG. 8). The effect of CTIP2 in such axonal fasciculation and extension defects appears specific to only distinct types of sub-cerebral and/or sub-cortical axons, since other fiber tracts (e.g. the corpus callosum) appear normal (FIGS. 8 A,D). This is consistent with our findings that callosal projection neurons do not normally express CTIP2 (FIGS. 6 and 12).

To more closely examine axonal projections of CSMN in ctip2−/− mice, we first compared the corticospinal fiber tracts of ctip2−/− mice to those of wild type littermates at P0, using immunocytochemistry for L1, a member of the CAM family of cell adhesion molecules that is known to be expressed by selected fiber tracts in the CNS, including CSMN projections (Fujimori et al., 2000). We find that P0 ctip2−/− mice lack the typical fasciculated bundles of sub-cerebral projections fibers that normally form the internal capsule. This phenotypic abnormality is very distinct and extends along the entire rostro-caudal axis of the internal capsule (FIGS. 8B,C,E,F). Moreover, high magnification analysis of L1-expressing fibers in the sagittal plane confirms this striking phenotype in ctip2−/− mice (FIGS. 8G,H,J,K). Interestingly, some of the highly disorganized and non-fasciculated ctip2−/− axonal projections deviate dramatically from their normal path (FIG. 8K), coursing obliquely and transverse to other axons of the internal capsule.

In order to illuminate the fine axonal architecture of the abnormal non-fasciculated internal capsule fibers in ctip2−/− mice, we performed anterograde DiI tracing of these fiber tracts by placing DiI crystals in the cortex of P0 ctip2−/− mice and wild type controls. High magnification laser confocal analysis of DiI-labeled fibers in the internal capsule further confirms this axon growth and fasciculation defect by showing that ctip2−/− axons are present as individual, non-fasciculated, and disorganized fibers (FIGS. 8 I,L). Some of the disorganized axons in the ctip2−/− mice possess what appear to be abnormal, bulbous varicosities and dysmorphic growth cones (FIG. 8L) suggestive of those first described by Ramon y Cajal (Ramon Y Cajal, 1928) and more recently highlighted and investigated by Silver and colleagues (Silver, 2004).

Given these striking abnormalities, we further examined the outgrowth of subcerebral axonal projections and the formation of the corticospinal tract in detail. We performed in vivo anterograde DiI tracing at P0 by injecting DiI into developing sensorimotor cortex of Ctip2^{-/-} mice and matched wild-type littermate controls. Close examination of axons along the length of the developing corticospinal tract revealed that, while approximately normal numbers of axons extend as far as the hypothalamus, they are disorganized, not normally fasciculated, and located dorsal to their normal position. Outgrowing axons also exhibit striking deviations from their normal path and extend toward ectopic targets (FIGS. 13A-13G). Only a small number of axons were observed caudal to the hypothalamus, and these were frequently extending in the wrong direction (FIGS. 13C and 13G). Most notably, no CSMN axons extended past the pons in Ctip2^{-/-} mice (n=7) (FIG. 13H), while CSMN axons in all wild-type and heterozygous littermates analyzed (n=20) extended normally through the medulla toward the pyramidal decussation and in some cases had already entered the spinal cord by P0 (FIG. 13D). Taken together, these data demonstrate that Ctip2 is critical and necessary for CSMN to extend projections to the spinal cord.

Given the extremely high levels of CTIP2 expression in subcerebral projection neurons, we examined heterozygous Ctip2^{+/-} mice to determine whether there is a gene dosage effect on the observed abnormalities. These experiments allowed us to investigate the role of CTIP2 into adulthood, much later than the P0 age at which Ctip2^{-/-} mice die. Interestingly, we found subtle defects in fasciculation in the internal capsule in Ctip2^{-/-} mice (data not shown), indicating a gene dosage effect.

To investigate the ability of Ctip2^{+/-} CSMN to properly establish and maintain projections to the spinal cord, we injected FG into the cervical spinal cord of 3- and 10-week-old Ctip2^{+/-} mice and quantified labeled CSMN in the entire cortex. During normal development, subcerebral neurons in layer V of lateral sensory cortex initially extend an axon to the spinal cord, but only a small percentage of these neurons maintain corticospinal projections into adulthood (Polleux et al., 2001). Quite remarkably, we find that, at 3 and 10 weeks of age, a large number of neurons in lateral sensory cortex of Ctip2^{+/-} mice aberrantly maintain ectopic projections to the spinal cord (at 3 weeks: wt 272±39, n=5; Ctip2^{+/-} 623±23, n=4; p=0.0002; at 10 weeks: wt 333±27, n=4; Ctip2^{+/-} 1088±403, n=5; p=0.14) (FIG. 14). In contrast, the number of neurons with spinal projections in sensorimotor cortex is the same in wild-type and Ctip2^{+/-} mice (at 3 weeks: wt 4767±507, n=5; Ctip2^{+/-} 4004±223, n=4; p=0.25; at 10 weeks: wt 3977±216, n=4; Ctip2^{+/-} 3911±454, n=5; p=0.91). These data indicate that ctip2 plays an important role in directing the developmental pruning and refinement of projections to the spinal cord.

Together, these results with both Ctip2^{-/-} and Ctip₂^{+/-} mice support the hypothesis that CTIP2 is centrally involved in orchestrating the complex extension, fasciculation, and refinement of subcerebral axonal projections and particularly the ability of CSMN to extend projections to the spinal cord during the formation of the corticospinal tract. This, in turn, may affect CSMN connectivity even before CSMN axons reach the medullary decussation and enter the spinal cord. Future experiments using chimeric and/or conditional ctip2−/− mice in which the corticospinal fiber tracts of the mice can be investigated at later postnatal stages could further test these hypotheses and extend these findings.

Discussion

One of the most striking characteristics of the nervous system is its remarkable cellular heterogeneity and anatomical complexity, largely due to the fact that neurons are among the most diversified populations of cells in the body. Despite the fact that a detailed morphologic classification of neuronal subtypes in different regions of the CNS has been available for a long time, our knowledge of the genes that control specification and differentiation of different CNS neuronal subtypes, with some important exceptions (Cepko, 1999; Wichterle et al., 2002), is substantially lacking.

This is especially true in the mammalian neocortex, where very little is known about the genes that control the progressive commitment and differentiation of immature progenitors to become selected subtypes of post-mitotic projection neurons. Progress has been slow, because attempts to study the developmental controls over neuronal subtypes have been hampered by the inability to distinguish different types of projection neurons with distinct and specific molecular markers.

Purification and Genetic Analysis of Distinct Neuronal Subpopulations

Here, we purified different populations of cortical projection neurons at distinct stages of development and studied the subtype-specific molecular controls over their development. Distinct classes of long-distance projection neurons were labeled based on their axonal projections, taking advantage of an intrinsic anatomical property (e.g. distant axonal fields) that is also shared by many other classes of projection neurons. These methods could thus be used to label and purify other neuronal subclasses in a systematic fashion. The use of an ultrasound-guided injection method also enables precise labeling of projection neurons, even at early stages of embryonic development.

Additionally, at each developmental stage studied, we were able to purify relatively homogeneous neuronal populations in fairly large numbers. This is advantageous for two reasons: 1) the initial RNA population undergoes relatively limited amplification during probe preparation, reducing the chances of introducing artifacts. This is supported by our finding that all biological replicate microarrays are highly correlated (Table 1); 2) it enhances the probability of identifying genes that are true common genetic determinants of the neuronal population sampled, rather than differentially expressed genes in only some of the neurons within the population. The depth and robustness of the data obtained using the neuronal populations purified in this manner is demonstrated by the fact that we identified large clusters of developmentally regulated genes that are highly correlated to known genes from the literature with similar temporal trends of expression, and by the fact that all eleven differentially expressed genes that we further investigated were confirmed by in situ hybridization or immunocytochemistry (FIGS. 5 and 11).

In the experiments described above, we specifically focused on the clinically relevant neuronal subtype of CSMN. We purified CSMN during embryonic (E18) and post-natal development (P3, P6, P14) and compared their gene expression profile to two related populations of purified cortical projection neurons of layer V, callosal neurons and corticotectal neurons, across the same developmental period. We hypothesized that, during CSMN development, there exist both (i) genes that are used by all cortical projection neurons and, therefore, may control general aspects of early projection neuron specification or later morphologic differentiation, and (ii) genes that are neuronal subtype-restricted and contribute to define the specific population of CSMN. Combinatorial interactions of both of these classes of genes, in the correct temporal order, is likely necessary to instruct neural precursors towards a CSMN-specific fate. By using pure populations of neurons for global gene expression analysis at multiple temporal stages, we were able to confirm our hypotheses and identified a large number of genes in both classes.

Common Determinants of Projection Neuron Differentiation

We identified genes in the first class, molecules important for general projection neuron differentiation, by cluster analysis. As shown in FIG. 3A, three major clusters of genes can be seen that show the same profile of expression in all neuronal subtypes analyzed and are clearly developmentally regulated. These genes likely are early (E18), intermediate (P3-P6), and late (P14) stage common determinants of projection neuron development (FIG. 3B-D). It is important that these clusters contain many genes that, at each stage, are known to control developmental events appropriate for the age analyzed, suggesting that each developmental stage-specific cluster contains genes of central biological relevance. For example, in the E18 cluster, largely uncharacterized, novel genes and ESTs cluster together with nestin, TGFβ2, and BMP6, all known to play important roles in early neuronal specification. Novel molecules in this cluster may play roles in the control of initial stages of projection neuron specification. Similarly, novel genes grouped in the P14 cluster are associated with known molecules like synaptophysin, synaptojanin 2, NMDA1, AMPA3, and AMPA1, all associated with circuit connectivity and synaptic function, suggesting that this second group of genes is likely enriched in molecules controlling mature aspects of projection neuron function. We propose that, together, these are important genetic determinants of general projection neuron specification and differentiation in vivo.

Identification of CSMN-specific Genes

Our investigation of genes in the second class, genes specific to CSMN, identified many genes that were not previously known to be expressed in CSMN (or in other specific classes of cortical neurons) and, thus, are novel genetic determinants of this neuronal subtype. These molecules are of particular interest, as they include genes that can be hypothesized to be involved in different aspects of CSMN development, from CSMN fate specification, to process outgrowth and axon guidance, to cell adhesion and survival. These are the critical developmental events that one might hypothesize should be controlled in a subtype-specific fashion, to direct a corticospinal motor neuron to (i) be born from a lineage-committed progenitor, (ii) migrate to the correct cortical layer, and (iii) survive long enough to (iv) be able to extend an axonal projection toward appropriate targets and (v) find synaptic partners.

Genes Implicated in CSMN-specific Early Fate Specification

At least two of the CSMN-specific genes among the ones that we further characterized may be novel early instructive signals of CSMN fate specification, as suggested by their specific expression in CSMN, as well as by recent reports suggesting fate-specification roles in other organisms. Fez, a six zinc finger domain-containing protein, is a particularly interesting candidate (Matsuo-Takasaki et al., 2000), since its zebrafish homolog was recently found to be involved in neuronal subtype fate specification (Levkowitz et al., 2003). This observation, combined with a distinct expression profile in CSMN and layer V in the mouse (FIG. 5D), strongly suggests that fez may be a CSMN specification factor during early development, and may be used to direct immature neuronal precursors toward a CSMN fate.

A second particularly interesting candidate in this regard is a largely uncharacterized gene in the mammalian CNS, clim1 (also known as Idb2), a cofactor that specifically interacts with transcription factors of the LIM-HD family, directly affecting their function (Becker et al., 2002). Here, we found that clim1 is highly expressed specifically in CSMN during early development (FIGS. 5F,F′). Although very little is known about the function of this gene, both its profile of expression and the fact that LIM proteins have established roles in cell-type specification during development (Bach, 2000; Jessell, 2000) are strong indications that this gene may control neuronal subtype specification in the CNS, particularly of CSMN.

Fez and clim1, together with other differentially expressed molecules identified here (FIGS. 3 and 4), are likely to be critical for specifying CSMN fate. We hypothesize that at least some of these molecules will be useful as markers to identify subtype-specific progenitors (if they exist) or cells committed to the CSMN fate soon after mitosis. This will be critical for connecting the pathways we present here with the extensive literature on initial neuronal specification (Anderson, 1999; Briscoe et al., 2000; Livesey and Cepko, 2001; Bertrand et al., 2002; Rallu et al., 2002; Shirasaki and Pfaff, 2002).

Genes Controlling CSMN Axon Outgrowth and Guidance

In order to potentially repair diseased corticospinal circuitry, it will likely be important to identify molecules that play central roles in CSMN axon outgrowth and pathfinding. Several such molecules specifically expressed in CSMN were identified in these experiments; each could play important roles in CSMN axon growth and guidance.

Ctip2 (also known as bcl11b) encodes a zinc finger DNA binding protein that binds DNA directly (Avram et al., 2002) and acts as a transcriptional repressor in vitro by recruiting SIRT1, a class III histone deacetylase, to specific target sequences (Senawong et al., 2003). While CTIP2 was initially discovered as an interacting partner of COUP-TF orphan nuclear receptors (Avram et al., 2000), it is unclear whether CTIP2 interacts with COUP-TF proteins in vivo (Senawong et al., 2003), or whether interaction with COUP-TFs is required for CTIP2-mediated gene expression. Loss of function experiments in vivo highlight an important role for CTIP2 in cell type specification in the immune system (Wakabayashi et al., 2003). No role for this gene in the nervous system was previously known. Here, we show that CTIP2 is expressed at high levels specifically in CSMN, while it is excluded from other types of projection neurons (e.g. callosal neurons). We further find that lack of CTIP2 expression in vivo in null mutant mice results in defects in the organization and fasciculation of sub-cerebral fiber tracts, including the CSMN axonal projections. This phenotype is particularly striking in the internal capsule, the path that CSMN axons follow during their initial outgrowth toward distal targets in the spinal cord. Our data are supported by the observation that COUP-TF1 null mutant mice also have defects in axon guidance and neuronal arborization (Qiu et al., 1997; Zhou et al., 1999). Taken together, these data, along with the specific expression of CTIP2 in CSMN and other sub-cerebral projection neurons of layer V, strongly suggest that CTIP2 is involved in the control of CSMN axonal extension and/or pathfinding.

Another of the CSMN-specific genes identified here, netrin-G1, encodes a recently discovered lipid-anchored axonal protein that contains elements of homology to two major classes of proteins involved in axon pathfinding in the CNS and PNS, the laminins and the netrins (Yin et al., 2002). Interestingly, netrin-G1 has been recently suggested to be involved in axon guidance of thalamocortical axons to their final targets in the cortex via interaction with its ligand, Netrin-G1 ligand (Lin et al., 2003). Most importantly, the same authors report expression of Netrin-G1 ligand in the medulla and spinal cord, which, together with our data showing high level expression of netrin-G1 in CSMN, suggest that netrin-G1 could mediate axonal outgrowth of CSMN to distinct targets in the spinal cord. Consistent with this hypothesis, we found that netrin-G1 expression remains low in CSMN until P6 and P14, when CSMN axons are finding targets in the spinal cord. To further investigate netrin-G1 function, it would be very informative to define the subcellular distribution of Netrin-G1 inside CSMN at different stages of axonal extension, and the specific localization of Netrin-G1 ligand in the spinal cord and medulla.

Genes Controlling CSMN Differentiation and Survival

Our genetic analysis also identified molecules whose expression profile and identity suggests they could be involved in later CSMN maturation and survival. IGFBP4 binds insulin-like growth factors and directly modulates IGF stability and action (Stenvers et al., 1994; Zhou et al., 2003). The established role of IGF on cell survival (D'Ercole et al., 1996; Stewart and Rotwein, 1996), combined with the fact that we found that IGFBP4 has a defined area-specific pattern of expression in the cortex, restricted to motor cortex and distinct from those of other IGFBPs, suggest that IGFBP4 may be a mediator of the effects of IGF in motor cortex. Together with previous data, our data support the hypothesis that IGFBP4 may control CSMN survival by extending IGF half-life specifically in these neurons.

mu-crystallin has one of the most striking profiles in the microarray data, exhibiting a dramatic increase in expression postnatally in CSMN and corticotectal neurons, while remaining at much lower levels during the development of callosal neurons (FIGS. 4 and 5G,G′). mu-crystallin is also known as CTBP (cytosolic T₃ binding protein) (Vie et al., 1997) and has a direct role in mediating the accumulation of T₃ (3,5,3′-triiodo-L-thyronine) in the cytoplasm and transport to the nucleus, therefore controlling T₃ mediated gene transactivation (Hashizume et al., 1989; Mori et al., 2002). Based on the interesting profile of subtype-restricted expression from our microarray data and in situ hybridization, and because thyroid hormone controls important aspects of neuronal differentiation and survival in the CNS (Oppenheimer and Schwartz, 1997), mu-crystallin could play a central role in T₃ mediated CSMN survival. Of additional interest, human mu-crystallin maps to chromosome 16 at a location near a newly identified locus for hereditary ALS (Sapp et al., 2003). The CSMN-specific expression of this gene, together with the central involvement of CSMN in ALS, suggests mu-crystallin as an interesting candidate gene for subtypes of hereditary ALS.

CSMN Repopulation and Repair of Corticospinal Circuitry

We have previously shown that distinct subtypes of neurons can be induced to undergo neurogenesis from immature precursors, even in the normally inhibitory environment of the adult mammalian neocortex (Magavi et al., 2000; Scharff et al., 2000). Although more recent data indicate that endogenous neural precursors can also be induced to differentiate into CSMN in vivo (Chen et al., unpublished observations), the number of newborn CSMN is quite low, and only a small percentage of the newborn neurons survive long enough to establish permanent connections to distal targets in the spinal cord. While these experiments demonstrate that new CSMN can be added to the normally inhibitory environment of the adult cortex and extend long-distance projections to the spinal cord, it is likely that the number of functional newborn CSMN could be substantially increased and functional recovery might be effected by improved understanding of controls over CSMN survival and connectivity at the molecular level. Such information might enhance survival of developing CSMN and improve the ability of CSMN to connect to proper targets, which in turn might enhance functional connectivity and circuit repair.

Alternatively, similar manipulation of key controls over CSMN development might enable the future directed differentiation of CSMN in vitro from neural precursors, toward production of functional CSMN for cellular transplantation. Such CSMN derived in vitro might be used to replace endogenous CSMN lost to neurodegeneration. Parallel experiments with similar motivations, targeting spinal “lower motor neurons” as a neuronal population for replacement, recently demonstrated production of lower motor neurons from ES cells (Wichterle et al., 2002).

In this report, we identify a large program of CSMN specific gene expression, and further study 10 genes that are specifically expressed in CSMN. We propose that these genes might, in the future, be manipulated to control distinct aspects of CSMN differentiation from immature precursor cells in vitro and/or in vivo. We speculate that: (i) known signals controlling general neuronal specification (e.g. ngn1/ngn2, pax6) might be integrated with (ii) genes that control generic neuronal differentiation (e.g. neuroD, math2), and with (iii) some of the newly identified genes (e.g. fez and clim1) to produce precursors that are committed to become layer V sub-cerebral projection neurons. These, in turn, might express a subsequent set of sub-cerebral projection neuron specification genes (e.g. ctip2, encephalopsin, pcp4, mu-crystallin, csmn1, and netrin-G1). Ultimately, the finer molecular distinction of CSMN from other very related classes of layer V sub-cerebral projection neurons might be defined by specific combinatorial interactions of these and other factors, rather than by single genes, as well as by the exclusion of genes specific to other neuronal subtypes (e.g. lmo4 for callosal neurons, lix1 for corticotectal neurons (Moeller et al., 2002), and tbr1 for corticothalamic projection neurons (Hevner et al., 2001). Additionally, the identification of genes expressed in CSMN in the sharply delimited region of motor cortex (e.g. igfbp4 and crim1) might also contribute to define and generate the CSMN population. Although this complex model will ultimately require investigation of the functional roles of individual and combinations of these genes in the context of CSMN development, the data presented here already support the idea that a more precise molecular classification of distinct classes of projection neurons is possible. Our results provide the foundation for increasingly sophisticated analysis and a detailed understanding of stage-specific genes controlling corticospinal motor neuron development.

### Example 2

### Fez Specifies Fate of CSMN

Towards the goal of identifying genes involved in specifying individual subtypes of projection neurons, we identified genes specific to corticospinal motor neurons (CSMN), clinically relevant cortical output neurons, which degenerate in amyotrophic lateral sclerosis (ALS), and whose injury contributes to the loss of motor function following spinal cord injury. As shown above in Example 1, we found genes specific to CSMN, as well as genes more broadly expressed in the highly related population of subcerebral projection neurons of layer V.

From this previous study, we sought to identify transcription factors that might be involved in the earliest events of CSMN fate specification. One molecule, Fez (Forebrain Expressed Zinc finger-Like; also called ZFP312), is a particularly promising candidate for several reasons. The pattern of Fez expression is consistent with a role in fate specification of subcerebral projection neurons. As shown above in Example 1, we found that Fez is expressed at a constant level in CSMN and other subcerebral projection neurons of layer V from E18.5 to P14. In addition, in situ hybridization studies during earlier stages of development indicate that Fez is first expressed as early as E8.5 in the dorsal telencephalic wall (Hirata, Dev Dyn, 2004). Later in development, between E12.5 and E14.5, at a time when critical events of subcerebral projection neuron fate specification likely occur, high level expression of Fez is detected in the ventricular zone in a pattern consistent with the location of neuronal progenitors, and in the developing cortical plate consistent with the position of new born subcerebral projection neurons.

All together these data suggest that Fez is expressed in the right time and place for it to be involved in subcerebral projection neuron (including CSMN) fate specification. In further support of a role of Fez in neuronal subtype fate specification, are the findings that during zebrafish forebrain development, loss of the Fez zebrafish homologue results in the specific loss of DA and 5HT neurons of the hypothalamus, while other neuronal populations are spared (Levkowitz, Nature Neurosci, 2003).

We hypothesized that Fez is required for the initial fate specification, migration and survival of CSMN and other subcerebral projection neurons. We examined mice in which the Fez gene had been genetically modified to alter the function of the gene. By multiple methods of analysis we found that Fez is a key specification factor for subcerebral projection neurons, which are defined herein as projection neurons located in layer 5 of the cortex that project to targets outside of the cerebrum (including spinal cord and brainstem, including targets in the red nucleus, tectum, pons, and medulla). In the absence of normal Fez levels and function, subcerebral projection neurons, including CSMN, are not specified and are not born from progenitor cells in the developing cerebral cortex.

These findings are supported by multiple methods of analysis which included: 1) using multiple anterograde and retrograde dyes for tracing specific axonal tracts including corticospinal axons, subcerebral axons, corticothalamic axons, interhemispheric cortico-cortical callosal axons, interhemispheric cortico-cortical anterior commissure axons, intrahemispheric cortico-cortical axons, corticoseptal axons and corticostriatal axons (Lanier et al., 1999; Mitchell et al., J Comp Neuro, 2005; Arlotta et al., Neuron, 2005; Hevner et al., 2001; O'Leary and Terashima, 1988; Godement et al., 1987); 2) multiple molecular marker analysis via in situ hybridization and immunocytochemistry of the differentiation of multiple distinct neuronal subtypes in all cortical layers using newly described markers of Example 1 as well as markers previously described (Arlotta et al., 2005; Hevner, Dev Neurosci, 2003); 3) cell death detection methods including FluoroJade (Histo-Chem, Jefferson, Ark.) and TUNEL (Arlotta et al., 2005); and 4) cortical neuronal migration analysis by in utero BrdU birthdating at multiple distinct stages of cortical neurogenesis (Hevner, Dev Neurosci, 2003).

These results definitively establish that Fez is centrally involved in orchestrating the early specification of subcerebral projection neurons.

### Example 3

### Ctip2 Involved in Development of Medium Spiny Projection Neurons of the Striatum

The medium spiny projection neurons of the striatum are one of the critical populations of neurons that degenerate in Huntington's disease. The identification of critical genes governing the differentiation, maturation and survival of medium spiny neurons is critical for developing new therapies 1) to prevent the degeneration of medium spiny neurons in Huntington's disease, 2) to recruit precursors and direct their differentiation into medium spiny neurons, and 3) to direct stem or progenitor cells in vitro to differentiation into medium spiny neurons for transplantation.

We observed that CTIP2 is expressed at extremely high levels in the striatum and areas of striatal neurogenesis throughout development. By analysis of multiple cell type specific markers using immunocytochemical methods, we found that within the striatum, CTIP2 is expressed specifically in medium spiny neurons. Because CTIP2 is known to be involved in maturation and survival of other cell types, we hypothesized that CTIP2 might play a key role in the development of striatal medium spiny neurons. We examined mice in which the CTIP2 gene had been genetically modified to alter the function of the gene. By multiple methods of analysis we found that CTIP2 is a critical molecule for the maturation, migration and survival of striatal medium spiny neurons. In the absence of normal CTIP2 levels and function, striatal medium spiny neurons do not mature properly, fail to migrate to their appropriate location in the striatum, and undergo premature cell death.

These findings are supported by multiple methods of analysis which included: 1) multiple molecular marker analysis via immunocytochemistry of the differentiation of multiple distinct neuronal subtypes within the striatum (Arlotta et al., 2005; Tamura et al., 2004; Walaas and Greengard, 1984); 2) cell death detection methods including FluoroJade, TUNEL, and cleaved caspase 3 immunoctyochemistry (Arlotta et al., 2005); and 3) striatal neuronal migration analysis by in utero BrdU birthdating at multiple distinct stages of striatal neurogenesis (Van der Kooy and Fishell, 1987; Krushel et al., 1995).

### Example 4

### Identification of Genes Specifically Expressed in Callosal Projection Neurons

Using methods similar to those described for the identification of genes specifically involved in the development of CSMN (FACS purification of projection neuron subtypes combined with microarray expression profiling), we identified genes specifically expressed in callosal projection neurons with precise, developmentally regulated expression profiles, which are listed in Table 6.

TABLE 6
Summary of Callosal Projection Neuron Specific Genes

| Accession # | Gene name |
|---|---|
| NM_010723 | Lmo4 |
| NM_008509 | Lipoprotein Lipase |
| BF178348 | pleiotrophin |
| NM_138749 | Plexin B2 |
| NM_018884 | Semcap3 |
| NM_010151 | Coup-TF1 |
| NM_010710 | LIM homeobox 2 |
| BB078052 | G-protein coupled |
| | receptor 88 |
| NM_008380 | inhibin beta-A |
| AI893646 | Ptprk |
| BG070704 | Pvrl3 |
| BC024515 | gastrin releasing |
| | peptide |
| AF183946 | cadherin 10 |
| M16120 | T-Cell Receptor Beta Variable 13 |
| BB292785 | Eph receptor A3 |
| AK017580 | 4930403J22Rik |
| BQ180367 | Vglut2 |
| BQ175659 | chimerin 2 |
| AK006371 | Nnmt |
| C88150 | Hnrpa2b1 |
| NM_001001980 | 3732412D22 |
| AW322026 | B-cell translocation gene 1 |
| BB009122 | Grsp1 |
| AK004853 | dickkopf homolog 3 |
| AB029929 | caveolin-1 |
| NM_019960 | Hspb3 |
| NM_007804 | cut-like 2 |
| NM_021272 | fabp7 |
| BC019530 | plexin D1 |
| Y15163 | Cited2 |

Callosal neurons are useful for treatments of diseases and disorders including Alzheimer's Disease, autism spectrum disorders, Rett Syndrome, and agenesis/dysgenesis/degereantion of the corpus callosum.

### REFERENCES

Anderson, D. J. (1999). Lineages and transcription factors in the specification of vertebrate primary sensory neurons. Curr Opin Neurobiol 9, 517-524.
Arber, S., Ladle, D. R., Lin, J. H., Frank, E., and Jessell, T. M. (2000). ETS gene Er81 controls the formation of functional connections between group Ia sensory afferents and motor neurons. Cell 101, 485-498.
Arlotta, P., Molyneaux, B. J., Chen, J., Inoue, J., Kominami, R., and Macklis, J. D. (2005). Neuronal subtype-specific genes that control corticospinal motor neuron development in vivo. Neuron 45, 207-221.
Avram, D., Fields, A., Pretty On Top, K., Nevrivy, D. J., Ishmael, J. E., and Leid, M. (2000). Isolation of a novel family of C(2)H(2) zinc finger proteins implicated in transcriptional repression mediated by chicken ovalbumin upstream promoter transcription factor (COUP-TF) orphan nuclear receptors. J Biol Chem 275, 10315-10322.
Avram, D., Fields, A., Senawong, T., Topark-Ngarm, A., and Leid, M. (2002). COUP-TF (chicken ovalbumin upstream promoter transcription factor)-interacting protein 1 (CTIP1) is a sequence-specific DNA binding protein. Biochem J 368, 555-563.
Bach, I., Carriere, C., Ostendorff, H. P., Andersen, B., and Rosenfeld, M. G. (1997). A family of LIM domain-associated cofactors confer transcriptional synergism between LIM and Otx homeodomain proteins. Genes Dev 11, 1370-1380.
Bach, I. (2000). The LIM domain: regulation by association. Mech Dev 91, 5-17.
Bai, W. Z., Ishida, M., and Arimatsu, Y. (2004). Chemically defined feedback connections from infragranular layers of sensory association cortices in the rat. Neuroscience 123, 257-267.
Barrett, M. T., Glogovac, J., Prevo, L. J., Reid, B. J., Porter, P., and Rabinovitch, P. S. (2002). High-quality RNA and DNA from flow cytometrically sorted human epithelial cells and tissues. Biotechniques 32, 888-890, 892, 894, 896.
Bayer, S. A., and Altman, J. (1991). Neocortical Development (New York, Raven Press).
Becker, T., Ostendorff, H. P., Bossenz, M., Schluter, A., Becker, C. G., Peirano, R. I., and Bach, I. (2002). Multiple functions of LIM domain-binding CLIM/NLI/Ldb cofactors during zebrafish development. Mech Dev 117, 75-85.
Berger, U. V., and Hediger, M. A. (2001). Differential distribution of the glutamate transporters GLT-1 and GLAST in tanycytes of the third ventricle. J Comp Neurol 433, 101-114.
Bertrand, N., Castro, D. S., and Guillemot, F. (2002). Proneural genes and the specification of neural cell types. Nat Rev Neurosci 3, 517-530.
Blackshaw, S., and Snyder, S. H. (1999). Encephalopsin: a novel mammalian extraretinal opsin discretely localized in the brain. J Neurosci 19, 3681-3690.
Bregman, B. S., Coumans, J. V., Dai, H. N., Kuhn, P. L., Lynskey, J., McAtee, M., and Sandhu, F. (2002). Transplants and neurotrophic factors increase regeneration and recovery of function after spinal cord injury. Prog Brain Res 137, 257-273.
Briscoe, J., Pierani, A., Jessell, T. M., and Ericson, J. (2000). A homeodomain protein code specifies progenitor cell identity and neuronal fate in the ventral neural tube. Cell 101, 435-445.
Briscoe, J., and Ericson, J. (2001). Specification of neuronal fates in the ventral neural tube. Curr Opin Neurobiol 11, 43-49.
Brummendorf, T., Kenwrick, S., and Rathjen, F. G. (1998). Neural cell recognition molecule L1: from cell biology to human hereditary brain malformations. Curr Opin Neurobiol 8, 87-97.
Bulchand, S., Subramanian, L., and Tole, S. (2003). Dynamic spatiotemporal expression of LIM genes and cofactors in the embryonic and postnatal cerebral cortex. Dev Dyn 226, 460-469.
Catapano, L. A., Arnold, M. W., Perez, F. A., and Macklis, J. D. (2001). Specific neurotrophic factors support the survival of cortical projection neurons at distinct stages of development. J Neurosci 21, 8863-8872.
Catapano, L. A., Arlotta, P., Cage, T. A. and Macklis, J. D. (2004). Stage-specific and opposing roles of BDNF, NT-3, and bFGF in differentiation of purified callosal projection neurons toward cellular repair of complex circuitry. in press.
Cepko, C. L. (1999). The roles of intrinsic and extrinsic cues and bHLH genes in the determination of retinal cell fates. Curr Opin Neurobiol 9, 37-46.
Coonan, J. R., Greferath, U., Messenger, J., Hartley, L., Murphy, M., Boyd, A. W., Dottori, M., Galea, M. P., and Bartlett, P. F. (2001). Development and reorganization of corticospinal projections in EphA4 deficient mice. J Comp Neurol 436, 248-262.
D'Ercole, A. J., Ye, P., Calikoglu, A. S., and Gutierrez-Ospina, G. (1996). The role of the insulin-like growth factors in the central nervous system. Mol Neurobiol 13, 227-255.
Eberwine, J., Yeh, H., Miyashiro, K., Cao, Y., Nair, S., Finnell, R., Zettel, M., and Coleman, P. (1992). Analysis of gene expression in single live neurons. Proc Natl Acad Sci USA 89, 3010-3014.
Edlund, T., and Jessell, T. M. (1999). Progression from extrinsic to intrinsic signaling in cell fate specification: a view from the nervous system. Cell 96, 211-224.
Eisen, M. B., Spellman, P. T., Brown, P. O., and Botstein, D. (1998). Cluster analysis and display of genome-wide expression patterns. Proc Natl Acad Sci USA 95, 14863-14868.
Frantz, G. D., Bohner, A. P., Akers, R. M., and McConnell, S. K. (1994). Regulation of the POU domain gene SCIP during cerebral cortical development. J Neurosci 14, 472-485.
Fricker-Gates, R. A., Shin, J. J., Tai, C. C., Catapano, L. A., and Macklis, J. D. (2002). Late-stage immature neocortical neurons reconstruct interhemispheric connections and form synaptic contacts with increased efficiency in adult mouse cortex undergoing targeted neurodegeneration. J Neurosci 22, 4045-4056.
Fujimori, K. E., Takeuchi, K., Yazaki, T., Uyemura, K., Nojyo, Y., and Tamamki, N. (2000). Expression of L1 and TAG-1 in the corticospinal, callosal, and hippocampal commissural neurons in the developing rat telencephalon as revealed by retrograde and in situ hybridization double labeling. J Comp Neurol 417, 275-288.
Geschwind, D. H. (2000). Mice, microarrays, and the genetic diversity of the brain. Proc Natl Acad Sci USA 97, 10676-10678.
Giehl, K. M. (2001). Trophic dependencies of rodent corticospinal neurons. Rev Neurosci 12, 79-94.
Godement, P., Vanselow, J., Thanos, S., and Bonhoeffer, F. (1987). A study in developing visual systems with a new method of staining neurones and their processes in fixed tissue. Development 101, 697-713.
Griffin, R. S., Mills, C. D., Costigan, M., and Woolf, C. J. (2003). Exploiting microarrays to reveal differential gene expression in the nervous system. Genome Biol 4, 105.
Hashizume, K., Miyamoto, T., Ichikawa, K., Yamauchi, K., Kobayashi, M., Sakurai, A., Ohtsuka, H., Nishii, Y., and Yamada, T. (1989). Purification and characterization of NADPH-dependent cytosolic 3,5,3′-triiodo-L-thyronine binding protein in rat kidney. J Biol Chem 264, 4857-4863.
Hevner, R. F., Shi, L., Justice, N., Hsueh, Y., Sheng, M., Smiga, S., Bulfone, A., Goffinet, A. M., Campagnoni, A. T., and Rubenstein, J. L. (2001). Tbr1 regulates differentiation of the preplate and layer 6. Neuron 29, 353-366.
Hevner, R. F., Daza, R. A., Rubenstein, J. L., Stunnenberg, H., Olavarria, J. F., and Englund, C. (2003). Beyond laminar fate: toward a molecular classification of cortical projection/pyramidal neurons. Dev Neurosci 25, 139-151.
Hirata, T., Suda, Y., Nakao, K., Narimatsu, M., Hirano, T., and Hibi, M. (2004). Zinc finger gene fez-like functions in the formation of subplate neurons and thalamocortical axons. Dev. Dyn. 230, 546-556.
Huh, G. S., Boulanger, L. M., Du, H., Riquelme, P. A., Brotz, T. M., and Shatz, C. J. (2000). Functional requirement for class I MHC in CNS development and plasticity. Science 290, 2155-2159.
Jessell, T. M. (2000). Neuronal specification in the spinal cord: inductive signals and transcriptional codes. Nat Rev Genet 1, 20-29.
Jones, E. G., Schreyer, D. J., and Wise, S. P. (1982). Growth and maturation of the rat corticospinal tract. Prog Brain Res 57, 361-379.
Joosten, E. A., Verhaagh, S., Martin, D., Robe, P., Franzen, R., Hooiveld, M., Doombos, R., Bar, P. R., and Moonen, G. (1996). Alpha-MSH stimulates neurite outgrowth of neonatal rat corticospinal neurons in vitro. Brain Res 736, 91-98.
Joosten, E. A., and Bar, D. P. (1999). Axon guidance of outgrowing corticospinal fibres in the rat. J Anat 194 (Pt 1), 15-32.
Junger, H., and Junger, W. G. (1998). CNTF and GDNF, but not NT-4, support corticospinal motor neuron growth via direct mechanisms. Neuroreport 9, 3749-3754.
Kania, A., and Jessell, T. M. (2003). Topographic motor projections in the limb imposed by LIM homeodomain protein regulation of ephrin-A:EphA interactions. Neuron 38, 581-596.
Koester, S. E., and O'Leary, D. D. (1993). Connectional distinction between callosal and subcortically projecting cortical neurons is determined prior to axon extension. Dev Biol 160, 1-14.
Kolle, G., Georgas, K., Holmes, G. P., Little, M. H., and Yamada, T. (2000). CRIM1, a novel gene encoding a cysteine-rich repeat protein, is developmentally regulated and implicated in vertebrate CNS development and organogenesis. Mech Dev 90, 181-193.
Krushel, L. A., Fishell, G., van der Kooy, D. (1995) Pattern formation in the mammalian forebrain: striatal patch and matrix neurons intermix prior to compartment formation. Eur J Neurosci. 7,1210-1219.
Lanier, L. M., Gates, M. A., Witke, W., Menzies, A. S., Wehman, A. M., Macklis, J. D., Kwiatkowski, D., Soriano, P., and Gertler, F. B. (1999). Mena is required for neurulation and commissure formation. Neuron 22, 313-325.
Lee, S. K., and Pfaff, S. L. (2003). Synchronization of neurogenesis and motor neuron specification by direct coupling of bHLH and homeodomain transcription factors. Neuron 38, 731-745.
Levkowitz, G., Zeller, J., Sirotkin, H. I., French, D., Schilbach, S., Hashimoto, H., Hibi, M., Talbot, W. S., and Rosenthal, A. (2003). Zinc finger protein too few controls the development of monoaminergic neurons. Nat Neurosci 6, 28-33.
Lin, J. C., Ho, W. H., Gurney, A., and Rosenthal, A. (2003). The netrin-G1 ligand NGL-1 promotes the outgrowth of thalamocortical axons. Nat Neurosci 6, 1270-1276.
Liu, J. P., Laufer, E., and Jessell, T. M. (2001). Assigning the positional identity of spinal motor neurons: rostrocaudal patterning of Hox-c expression by FGFs, Gdf11, and retinoids. Neuron 32, 997-1012.
Liu, Q., Dwyer, N. D., and O'Leary, D. D. (2000). Differential expression of COUP-TFI, CHL1, and two novel genes in developing neocortex identified by differential display PCR. J Neurosci 20, 7682-7690.
Livesey, F. J., and Cepko, C. L. (2001). Vertebrate neural cell-fate determination: lessons from the retina. Nat Rev Neurosci 2, 109-118.
Lockhart, D. J., and Barlow, C. (2001). Expressing what's on your mind: DNA arrays and the brain. Nat Rev Neurosci 2, 63-68.
Luo, Z., and Geschwind, D. H. (2001). Microarray applications in neuroscience. Neurobiol Dis 8, 183-193.
Magavi, S. S., Leavitt, B. R., and Macklis, J. D. (2000). Induction of neurogenesis in the neocortex of adult mice. Nature 405, 951-955.
Matsuo-Takasaki, M., Lim, J. H., Beanan, M. J., Sato, S. M., and Sargent, T. D. (2000). Cloning and expression of a novel zinc finger gene, Fez, transcribed in the forebrain of Xenopus and mouse embryos. Mech Dev 93, 201-204.
Mitchell B. D., and Macklis J. D. (2005). Large-scale maintenance of dual projections by callosal and frontal cortical projection neurons in adult mice. J Comp Neurol. 482(1), 17-32.
Moeller, C., Yaylaoglu, M. B., Alvarez-Bolado, G., Thaller, C., and Eichele, G. (2002). Murine Lix1, a novel marker for substantia nigra, cortical layer 5, and hindbrain structures. Brain Res Gene Expr Patterns 1, 199-203.
Mori, J., Suzuki, S., Kobayashi, M., Inagaki, T., Komatsu, A., Takeda, T., Miyamoto, T., Ichikawa, K., and Hashizume, K. (2002). Nicotinamide adenine dinucleotide phosphate-dependent cytosolic T(3) binding protein as a regulator for T(3)-mediated transactivation. Endocrinology 143, 1538-1544.
Nakagawa, Y., Johnson, J. E., and O'Leary, D. D. (1999). Graded and areal expression patterns of regulatory genes and cadherins in embryonic neocortex independent of thalamocortical input. J Neurosci 19, 10877-10885.
Novitch, B. G., Wichterle, H., Jessell, T. M., and Sockanathan, S. (2003). A requirement for retinoic acid-mediated transcriptional activation in ventral neural patterning and motor neuron specification. Neuron 40, 81-95.
O'Leary, D. D., and Koester, S. E. (1993). Development of projection neuron types, axon pathways, and patterned connections of the mammalian cortex. Neuron 10, 991-1006.
O'Leary, D. D., and Nakagawa, Y. (2002). Patterning centers, regulatory genes and extrinsic mechanisms controlling arealization of the neocortex. Curr Opin Neurobiol 12, 14-25.
O'Leary, D. D., and Terashima, T. (1988). Cortical axons branch to multiple subcortical targets by interstitial axon budding: implications for target recognition and “waiting periods”. Neuron 1, 901-910.
Oppenheimer, J. H., and Schwartz, H. L. (1997). Molecular basis of thyroid hormone-dependent brain development. Endocr Rev 18, 462-475.
Polleux, F., Dehay, C., Goffinet, A., and Kennedy, H. (2001). Pre- and post-mitotic events contribute to the progressive acquisition of area-specific connectional fate in the neocortex. Cereb. Cortex 11, 1027-1039.
Qiu, Y., Pereira, F. A., DeMayo, F. J., Lydon, J. P., Tsai, S. Y., and Tsai, M. J. (1997). Null mutation of mCOUP-TFI results in defects in morphogenesis of the glossopharyngeal ganglion, axonal projection, and arborization. Genes Dev 11, 1925-1937.
Ragsdale, C. W., and Grove, E. A. (2001). Patterning the mammalian cerebral cortex. Curr Opin Neurobiol 11, 50-58.
Rakic, P. (2002). Evolving concepts of cortical radial and areal specification. Prog Brain Res 136, 265-280.
Rallu, M., Corbin, J. G., and Fishell, G. (2002). Parsing the prosencephalon. Nat Rev Neurosci 3, 943-951.
Ramon Y Cajal, S. (1928). In Recuerdos de mi vida, pp. 516-546.
Rolf, B., Bastmeyer, M., Schachner, M., and Bartsch, U. (2002). Pathfinding errors of corticospinal axons in neural cell adhesion molecule-deficient mice. J Neurosci 22, 8357-8362.
Sangameswaran, L., Hempstead, J., and Morgan, J. I. (1989). Molecular cloning of a neuron-specific transcript and its regulation during normal and aberrant cerebellar development. Proc Natl Acad Sci USA 86, 5651-5655.
Sapp, P. C., Hosler, B. A., McKenna-Yasek, D., Chin, W., Gann, A., Genise, H., Gorenstein, J., Huang, M., Sailer, W., Scheffler, M., et al. (2003). Identification of two novel loci for dominantly inherited familial amyotrophic lateral sclerosis. Am J Hum Genet 73, 397-403.
Scharff, C., Kim, J. R., Grossman, M., Macklis, J. D., and Nottebohm, F. (2000). Targeted neuronal death affects neuronal replacement and vocal behavior in adult songbirds. Neuron 25, 481-492.
Segovia, L., Horwitz, J., Gasser, R., and Wistow, G. (1997). Two roles for mu-crystallin: a lens structural protein in diurnal marsupials and a possible enzyme in mammalian retinas. Mol Vis 3, 9.
Senawong, T., Peterson, V. J., Avram, D., Shepherd, D. M., Frye, R. A., Minucci, S., and Leid, M. (2003). Involvement of the histone deacetylase SIRT1 in chicken ovalbumin upstream promoter transcription factor (COUP-TF)-interacting protein 2-mediated transcriptional repression. J Biol Chem 278, 43041-43050.
Shirasaki, R., and Pfaff, S. L. (2002). Transcriptional codes and the control of neuronal identity. Annu Rev Neurosci 25, 251-281.
Silver, J. a. M. J. H. (2004). Regeneration beyond the glial scar. Nat Rev Neurosci 5, 146-156.
Stanfield, B. B., O'Leary, D. D., and Fricks, C. (1982). Selective collateral elimination in early postnatal development restricts cortical distribution of rat pyramidal tract neurones. Nature 298, 371-373.
Stenvers, K. L., Zimmermann, E. M., Gallagher, M., and Lund, P. K. (1994). Expression of insulin-like growth factor binding protein-4 and -5 mRNAs in adult rat forebrain. J Comp Neurol 339, 91-105.
Stewart, C. E., and Rotwein, P. (1996). Growth, differentiation, and survival: multiple physiological functions for insulin-like growth factors. Physiol Rev 76, 1005-1026.
Tamura, S., Morikawa, Y., Iwanishi, H., Hisaoka, T., Senba, E. (2004). Foxp1 gene expression in projection neurons of the mouse striatum. Neuroscience 124, 261-267.
Terashima, T. (1995). Anatomy, development and lesion-induced plasticity of rodent corticospinal tract. Neurosci Res 22, 139-161.
Tusher, V. G., Tibshirani, R., and Chu, G. (2001). Significance analysis of microarrays applied to the ionizing radiation response. Proc Natl Acad Sci USA 98, 5116-5121.
Van der Kooy, D., Fishell, G. (1987). Neuronal birthdate underlies the development of striatal compartments. Brain Res 401, 155-161.
Vie, M. P., Evrard, C., Osty, J., Breton-Gilet, A., Blanchet, P., Pomerance, M., Rouget, P., Francon, J., and Blondeau, J. P. (1997). Purification, molecular cloning, and functional expression of the human nicodinamide-adenine dinucleotide phosphate-regulated thyroid hormone-binding protein. Mol Endocrinol 11, 1728-1736.
Wakabayashi, Y., Watanabe, H., Inoue, J., Takeda, N., Sakata, J., Mishima, Y., Hitomi, J., Yamamoto, T., Utsuyama, M., Niwa, O., et al. (2003). Bcl11b is required for differentiation and survival of alphabeta T lymphocytes. Nat Immunol 4, 533-539.
Walaas, S. I., Greengard, P. (1984) DARPP-32, a dopamine- and adenosine 3′:5′-monophosphate-regulated phosphoprotein enriched in dopamine-innervated brain regions. I. Regional and cellular distribution in the rat brain. J Neurosci 4, 84-98.
Weimann, J. M., Zhang, Y. A., Levin, M. E., Devine, W. P., Brulet, P., and McConnell, S. K. (1999). Cortical neurons require Otx1 for the refinement of exuberant axonal projections to subcortical targets. Neuron 24, 819-831.
Wichterle, H., Lieberam, I., Porter, J. A., and Jessell, T. M. (2002). Directed differentiation of embryonic stem cells into motor neurons. Cell 110, 385-397.
Yin, Y., Miner, J. H., and Sanes, J. R. (2002). Laminets: laminin- and netrin-related genes expressed in distinct neuronal subsets. Mol Cell Neurosci 19, 344-358.
Zhou, C., Qiu, Y., Pereira, F. A., Crair, M. C., Tsai, S. Y., and Tsai, M. J. (1999). The nuclear orphan receptor COUP-TFI is required for differentiation of subplate neurons and guidance of thalamocortical axons. Neuron 24, 847-859.
Zhou, R., Diehl, D., Hoeflich, A., Lahm, H., and Wolf, E. (2003). IGF-binding protein-4: biochemical characteristics and functional consequences. J Endocrinol 178, 177-193.
   Equivalents

Each of the foregoing patents, patent applications, referenced sequences (as known at the filing date of this application) and other references is hereby incorporated by reference.

While the invention has been described with respect to certain embodiments, it should be appreciated that many modifications and changes may be made by those of ordinary skill in the art without departing from the spirit of the invention. It is intended that such modification, changes and equivalents fall within the scope of the following claims.

1. A method for differentiating cells to corticospinal motor neurons (CSMN), comprising
modulating the activity of one or more CSMN fate specification or end stage differentiation gene products by contacting a population of stem cells, neural and/or neuronal progenitors or precursors with a molecule that modulates expression of one or more CSMN fate specification or end stage differentiation gene products.

2. A method for differentiating cells to corticospinal motor neurons (CSMN), comprising
modulating the activity of one or more CSMN fate specification or end stage differentiation gene products by contacting a population of stem cells, neural and/or neuronal progenitors or precursors with a molecule that is a ligand, activator or repressor of the one or more CSMN fate specification or end stage differentiation gene products.

3. A method for promoting growth of corticospinal motor neurons (CSMN) axons in situ or in culture, comprising
modulating the activity of one or more CSMN axon guidance/process outgrowth promoting gene products by contacting a population of CSMN with a molecule that modulates expression of one or more CSMN axon guidance/process outgrowth promoting gene products that contribute to axon growth.

4. A method for inhibiting, preventing or reversing degeneration of corticospinal motor neurons (CSMN) axons in situ or in culture, or promoting CSMN survival in situ or in culture, comprising
modulating the activity of one or more CSMN survival gene products by contacting a population of CSMN with a molecule that modulates expression of one or more CSMN survival gene products that contribute to CSMN survival.

5-7. (canceled)

8. The method of claim 1, wherein the one or more gene products is the expression product of the fez clim1, ctip2, encephalopsin, pcp4, mu-crystallin, csmn1, igfb4, crim1 and/or netrin-G1 genes.

9. The method of claim 2, wherein the one or more gene products is the expression product of the fez, clim1, ctip2, encephalopsin, pcp4, mu-crystallin, csmn1, igfb4, crim1 and/or netrin-G1 genes.

10. (canceled)

11. The method of claim 3, wherein the one or more gene products is the expression product of the netrin-G1 and/or ctip2 genes.

12. (canceled)

13. The method of claim 4, wherein the one or more gene products is the expression product of the ctip2, igb4 and/or mu-crystallin genes.

14-18. (canceled)

19. A method of cell transplantation comprising
differentiating or promoting growth of CSMN as claimed in claims 1-4,
exposing the cell in vitro to cell growth conditions to form an expanded CSMN cell population, and
administering an amount of the expanded CSMN population or progeny cells produced therefrom to a patient.

20-30. (canceled)

31. A method for identifying lead compounds for a pharmacological agent useful in the differentiation of stem cells, neural and/or neuronal progenitors or precursors to corticospinal motor neurons (CSMN), comprising
contacting a population of stem cells, neural and/or neuronal progenitors or precursors with a candidate pharmacological agent under conditions that, in the absence of the candidate pharmacological agent, result in a baseline amount of expression of one or more CSMN-specific gene products; and
determining a test amount of expression of the one or more CSMN-specific gene products as a measure of the effect of the pharmacological agent on the expression of the one or more CSMN-specific gene products,
wherein a test amount of expression of the one or more CSMN-specific gene products that is greater than the baseline amount indicates that the candidate pharmacological agent is a lead compound for a pharmacological agent that is useful in the differentiation of stem cells, neural and/or neuronal progenitors or precursors to corticospinal motor neurons.

32-40. (canceled)

41. The method of claim 32, wherein the one or more gene products is the expression product of the fez and/or clim1 genes.

42. The method of claim 32, wherein the one or more gene products is the expression product of the ctip2, encephalopsin, pcp4, mu-crystallin, csmn1, igfb4, crim1 and/or netrin-G1 genes.

43. (canceled)

44. The method of claim 33, wherein the one or more gene products is the expression product of the netrin-G1 and/or ctip2 genes.

45. (canceled)

46. The method of claim 34, wherein the one or more gene products is the expression product of the ctip2, igfb4 and/or mu-crystallin genes.

47. A method for identifying corticospinal motor neurons (CSMN) in a biological sample, comprising
obtaining a biological sample comprising cells, and
analyzing the cells of the biological sample for the presence or expression of one or more CSMN-specific gene products,
wherein the presence or expression of the one or more CSMN-specific gene products is indicative of CSMN in the biological sample.

48-50. (canceled)

51. The method of claim 47, wherein the one or more gene products is the expression product of the fez and/or clim1 genes.

52. The method of claim 47, wherein the one or more gene products is the expression product of the ctip2, encephalopsin, pcp4, mu-crystallin, csmn1, igfb4, crim1 and/or netrin-G1 genes.

53. (canceled)

54. The method of claim 47, wherein the one or more gene products is the expression product of the netrin-G1 and/or ctip2 genes.

55. (canceled)

56. The method of claim 47, wherein the one or more gene products is the expression product of the ctip2, igfb4 and/or mu-crystallin genes.

57-61. (canceled)

62. A method for identifying corticospinal motor neurons (CSMN) in a biological sample, comprising
obtaining a biological sample comprising cells, and
analyzing the cells of the biological sample for the presence or expression of one or more CSMN-excluded gene products,
wherein the absence of the one or more CSMN-excluded gene products is indicative of CSMN in the biological sample.

63-65. (canceled)

66. A method for isolating a substantially pure population of corticospinal motor neurons (CSMN), comprising
selectively labeling a population of cells comprising CSMN by introducing a detectable marker into the CSMN neurons, and
isolating the labeled CSMN from unlabeled cells of the population by dissecting the CSMN neurons from other subtypes of neurons, and by sorting the labeled CSMN neurons to obtain a substantially pure population of the CSMN neurons.

67. The method of claim 66, wherein the labeling step is retrograde labeling comprising microinjecting the cells in a CNS structure that is a target of CSMN at the developmental stage of the organism at the time the injection is performed.

68. The method of claim 67, wherein the CNS structure is the pons-midbrain junction for embryos, or the pons or the cervical spinal cord at the C2-3 or C5 level for postnatal organisms.

69-75. (canceled)

76. The method of claim 66, wherein the detectable marker is a detectable gene product under the control of a promoter of a CSMN fate specification or end stage differentiation gene product, a CSMN axon guidance/process outgrowth promoting gene product, or a CSMN survival gene product, and further comprising selecting or sorting the CSMN based on the expression of the detectable molecule.

77-78. (canceled)

79. A method for isolating a substantially pure population of CSMN neurons, comprising
contacting a population of cells comprising CSMN with a molecule that binds selectively to a CSMN fate specification or end stage differentiation gene product, a CSMN axon guidance/process outgrowth promoting gene product, or a CSMN survival gene product, and
isolating the CSMN from unlabeled cells of the population by isolating the CSMN bound to the a molecule that binds selectively to obtain a substantially pure population of the CSMN.

80-100. (canceled)

101. A method for identifying lead compounds for a pharmacological agent useful in supporting the growth and/or survival of corticospinal motor neurons (CSMN), comprising
contacting a population of CSMN with a candidate pharmacological agent under conditions that, in the absence of the candidate pharmacological agent, result in a baseline amount of growth or survival of the CSMN; and
determining a test amount of growth or survival of the CSMN in the presence of the pharmacological agent as a measure of the effect of the pharmacological agent on the growth or survival of the CSMN,
wherein a test amount of growth or survival of the CSMN that is greater than the baseline amount indicates that the candidate pharmacological agent is a lead compound for a pharmacological agent that is useful in supporting the growth and/or survival of corticospinal motor neurons.

102-106. (canceled)
